SEQUENCE LISTING (13 sequences)

SEQ ID NO: 1 - protein, 124 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 2 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 3 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 107 aa, synthetic construct [sequence omitted]

SEQ ID NO: 5 - protein, 5 aa, synthetic construct
SFGMH

SEQ ID NO: 6 - protein, 17 aa, synthetic construct
YISSGSSTIYYGDTVKG

SEQ ID NO: 7 - protein, 15 aa, synthetic construct
EGGYYYGRSYYTMDY

SEQ ID NO: 8 - protein, 16 aa, synthetic construct
RSSQSIVHSNGNTYLE

SEQ ID NO: 9 - protein, 7 aa, synthetic construct
KVSNRFS

SEQ ID NO: 10 - protein, 9 aa, synthetic construct
FQGSHVPPT

SEQ ID NO: 11 - protein, 42 aa, unidentified [sequence omitted]

SEQ ID NO: 12 - protein, 40 aa, unidentified
DAEFRHDSGYEVHHQKLVFFAEDVGSNKGAIIGLMVGGVV

SEQ ID NO: 13 - protein, 673 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0352109 A1
(43) Pub. Date: Oct. 24, 2024

(54) SUBCUTANEOUS FORMULATIONS OF ANTI-ABETA PROTOFIBRIL ANTIBODY AND METHODS OF USE THEREOF

(71) Applicant: Eisai R&D Management Co., Ltd., Tokyo (JP)

(72) Inventors: Akihiko Koyama, Woodcliff Lake, NJ (US); Chad Swanson, Woodcliff Lake, NJ (US); Michio KANEKIYO, Woodcliff Lake, NJ (US); Michael IRIZARRY, Woodcliff Lake, NJ (US); Lynn Kramer, Woodcliff Lake, NJ (US); June KAPLOW, Woodcliff Lake, NJ (US); David VERBEL, Woodcliff Lake, NJ (US); Shobha DHADDA, Woodcliff Lake, NJ (US); Pallavi SACHDEV, Woodcliff Lake, NJ (US); Larisa REYDERMAN, Woodcliff Lake, NJ (US); Ishani Landry, Woodcliff Lake, NJ (US); Seiichi HAYATO, Tokyo (JP); Robert Gordon, Woodcliff Lake, NJ (US)

(21) Appl. No.: 18/688,264

(22) PCT Filed: Aug. 29, 2022
(86) PCT No.: PCT/US2022/041926

§ 371 Date: Feb. 29, 2024

Related U.S. Application Data
(60) Provisional application No. 63/260,730, filed on Aug. 30, 2021.
(60) Provisional application No. 63/306,050, filed on Feb. 2, 2022.
(60) Provisional application No. 63/269,389, filed on Mar. 15, 2022.
(60) Provisional application No. 63/269,463, filed on Mar. 16, 2022.
(60) Provisional application No. 63/364,619, filed on May 12, 2022.

(51) Int. Cl. C07K 16/18; A61K 9/00; A61K 39/00; A61K 47/18; A61K 47/26; A61P 25/28

(52) U.S. Cl. CPC C07K 16/18; A61K 9/0019; A61K 47/183; A61K 47/26; A61P 25/28; A61K 2039/505; A61K 2039/545

(57) ABSTRACT

Disclosed herein are methods of treating Alzheimer's disease, methods of reducing clinical decline in a subject having early Alzheimer's disease, methods of reducing brain amyloid level in a subject, methods of converting a subject from amyloid positive to amyloid negative, methods of preventing Alzheimer's disease, the methods comprising subcutaneously administering an anti-Aβ protofibril antibody.

This application claims the benefit of and priority to U.S. Provisional Applications 63/260,730 filed Aug. 30, 2021; 63/306,050 filed Feb. 2, 2022; 63/269,389 filed Mar. 15, 2022; 63/269,463 filed Mar. 16, 2022; and 63/364,619 filed May 12, 2022; each entitled “SUBCUTANEOUS FORMULATIONS OF ANTI-ABETA PROTOFIBRIL ANTIBODY AND METHODS OF USE THEREOF,” the contents of which are expressly incorporated herein by reference in their entirety.

This invention was partially made with government support under Grant Nos. R01AG054029, R01AG061848, and 5U24AG057437-04, awarded by the National Institutes of Health. The government has certain rights in this invention.

Alzheimer's disease (AD) is a progressive, neurodegenerative disorder of unknown etiology and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (2010 Alzheimer's disease facts and figures. Alzheimer Dement. 2010; 6:158-94). By the year 2050, the worldwide prevalence of AD is predicted to grow to 106.8 million (range: 47.2-221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage subjects bedridden. AD subjects typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD. (2010 Alzheimer's disease facts and figures, supra.)

Histologically, the disease is characterized by neuritic plaques, found primarily in the association cortex, limbic system and basal ganglia. The major constituent of these plaques is amyloid beta peptide (Aβ). Aβ exists in various conformational states—monomers, oligomers, protofibrils, and insoluble fibrils. Details of the mechanistic relationship between onset of Alzheimer's disease and AR production is unknown. However, some anti-Aβ antibodies are undergoing clinical study now as potential therapeutic agents for Alzheimer's disease.

Anti-Aβ antibodies and other proteins may be administered to subjects via intravenous, subcutaneous, intramuscular, and other means. The dosage, dosage form, and route of administration of an antibody can present many challenges.

Provided herein are methods for treating and/or preventing Alzheimer's disease comprising subcutaneously administering to a subject in need thereof an anti-Aβ protofibril antibody. Also provided herein are methods of reducing clinical decline in a subject having early Alzheimer's disease, methods of reducing brain amyloid level in a subject, and methods of converting a subject from amyloid positive to amyloid negative comprising subcutaneously administering to a subject in need thereof an anti-Aβ protofibril antibody. In some embodiments, the anti-Aβ protofibril antibody comprises a heavy chain variable regions comprising an amino acid sequence of SEQ ID NO: 1, and a light chain variable region comprising an amino acid sequence of SEQ ID NO: 2.

### BRIEF DESCRIPTION OF THE DRAWINGS

FIG. 1 depicts the 4 different dorsal injection location on the cynomolgus monkeys.

FIG. 2 depicts prerandomization and randomization schedules of the study disclosed in Examples 4 and 5.

FIG. 3 depicts a plot comparing the serum concentration over time of the IV formulation and the SC formulation.

FIG. 4 depicts a chart comparing the dose normalized Area Under the Curve (AUC) of the IV formulation and the SC formulation.

FIG. 5 depicts a plot of predicted serum concentration over 12 weeks of the IV formulation and the SC formulation (550 mg QW).

FIG. 6 depicts predicted 90% confidence intervals for AUCₛₛ geometric mean ratio comparison in healthy subjects from a single dose of the SC formulation based on simulated data.

FIG. 7 depicts a plot of predicted serum concentration over 12 weeks of the IV formulation and the SC formulation (720 mg QW).

FIG. 8 depicts a plot comparing the AUC in relation to body weight (BW in kg) of the IV formulation and the SC formulation. 10 mg/kg/BW iv and 720 mg/W sc refer to 10 mg/kg biweekly intravenous and 720 mg weekly subcutaneous, respectively.

FIG. 9 depicts a plot of the ratio of AUCsc/AUCiv in relation to body weight (BW in kg).

FIG. 10 depicts amyloid PET clearance in three graphs plotting PET SUVr over 18 months for 3 subjects of different bodyweights (51 kg, 70 kg, and 99 kg) having been administered the IV formulation and the SC formulation.

FIG. 11 depicts a graph of % change from baseline (CFB) in Global Cortical Average subcortical white matter (SWM) Standardized Uptake Value Ratio (SUVr) at 12 and 18 months in a predicted model.

FIG. 12 depicts a plot of predicted ARIA-E incidence (%) over the Cₘₐₓ.

FIG. 13 depicts two graphs of the predicted ARIA-E incidence (%) over 18 months of treatment with ApoE4 positive and ApoE4 negative subjects of different BW and being administered IV formulation and the SC formulation (550 mg QW).

FIG. 14 depicts two graphs of the predicted ARIA-E incidence (%) over 18 months of treatment with ApoE4 positive and ApoE4 negative subjects of different BW and being administered IV formulation and the SC formulation (720 mg QW).

FIG. 15 depicts the schedule of Example 5.

FIG. 16 depicts a brain autopsy gross section from Example 6.

FIG. 17 depicts representative axial and coronal florbetapir PET SUVr images showing progressive clearance of amyloid over time. (SUVR: Standardized uptake value ratio; CL: Centiloid unit; OLE: Open label extension; Top row: Baseline MRI; Rows 2-5: Florbetapir PET SUVR images at Baseline, weeks 55, 79 and 171 (OLE Baseline), respectively)

FIG. 18 depicts line plots of clinical scales during the course of the core phase, during which the patient received lecanemab 10 mg/kg IV biweekly for 79 weeks, separated by a gap period of 92 weeks without lecanemab treatment. The clinical scales assessed were MMSE, ADAS-cog, CDR-SB, and ADCOMS.

FIG. 19 depicts line plots of biomarkers during the course of the core phase, during which the patient received lecanemab 10 mg/kg IV biweekly for 79 weeks, separated by a gap period of 92 weeks without lecanemab treatment. The biomarkers assessed were amyloid PET, plasma Ab42/40 ratio (C2N assay), plasma p-tau181, and volumetric MRI.

FIG. 20 depicts microscope pictures at 12.5 times and 200 times magnification of the superior frontal cortex BA8,9 of a patient treated with lecanemab stained beta-amyloid, tau-AT8, and GFAP.

FIG. 21 depicts microscope pictures at 12.5 times and 200 times magnification of the superior frontal cortex BA8,9 of an untreated AD patient stained beta-amyloid and tau-AT8.

FIG. 22 depicts microscope pictures at 12.5 times and 200 times magnification of the hippocampal formation of a patient treated with lecanemab stained beta-amyloid, tau-AT8, and GFAP.

FIG. 23 depicts microscope pictures at 12.5 times and 200 times magnification of the hippocampal formation of an untreated AD patient stained beta-amyloid and tau-AT8.

FIG. 24 depicts microscope pictures at 400 times magnification of brain tissue stained for amyloid plaques in a patient treated with lecanemab (top) compared to an untreated AD patient.

FIG. 25 depicts microscope pictures of microglia in brain tissue by CD68 staining in a patient treated with lecanemab.

### DEFINITIONS

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B,” when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “adjusted mean change from baseline” refers to the use of a statistical analysis to calculate the change in a biomarker value over time. In some embodiments, a linear mixed-effects model (MMRM) is used to account for at least one additional covariate to determine the adjusted mean change from baseline.

When a number is recited, either alone or as part of a numerical range, it should be understood that the numerical value can vary above and below the stated value by up to a variance of +/−10% of the stated value. When a range of values is listed herein, it is intended to encompass each value and sub-range within that range. For example, “2.5 mg/kg to 10 mg/kg” is intended to encompass, for example, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 2.5 mg/kg to 3 mg/kg, 2.5 mg/kg to 4.5 mg/kg, 3 mg/kg to 4.5 mg/kg, 4.5 mg/kg to 8 mg/kg, 2.5 mg/kg to 9 mg/kg, and so forth.

Amyloid β 1-42 (Aβ 42) refers to an amyloid beta monomer from amino acid 1 to 42 of the full-length protein (Table 22, SEQ ID NO: 11). Amyloid β 1-40 (Aβ1-40) refers to an amyloid beta monomer from amino acid 1 to 40 of the full-length protein (Table 22, SEQ ID NO: 12).

Patients with “preclinical AD” or “pre-AD,” as described herein, are cognitively normal individuals with intermediate or elevated levels of amyloid in the brain and can be identified by asymptomatic stages with or without memory complaints and emerging episodic memory and executive function deficits. Cognitively normal can include individuals who are CDR 0, or individuals within the normal ranges of cognitive test scores (MMSE, International Shopping List Task, Logical Memory, etc.). Preclinical AD occurs prior to significant irreversible neurodegeneration and cognitive impairment and is typically characterized by the appearance of in vivo molecular biomarkers of AD and the absence clinical symptoms. Preclinical AD biomarkers that may suggest the future development of Alzheimer's disease include, but are not limited to, one or more intermediate or elevated levels of amyloid in the brain by amyloid or tau positron emission tomography (PET) (e.g., a centiloid measure of about 20-40, e.g., a measure of about 20-32), cerebrospinal fluid level of Aβ 1-42, cerebrospinal fluid level of total tau, cerebrospinal fluid level of neurogranin, cerebrospinal fluid level of neurofilament light chain, and blood biomarkers as measured in the serum or plasma (e.g. levels of βP1-42, the ratio of two forms of amyloid-β peptide (Aβ42/Aβ40, e.g., a ratio of between about 0.092-0.094 or below about 0.092), plasma levels of plasma total tau (T-tau), levels of phosphorylated tau (P-tau) isoforms (including tau phosphorylated at 181 (P-tau181) 217 (P-tau217), and 231 (P-tau231)), glial fibrillary acidic protein (GFAP), and neurofilament light (NfL)). For example, it has been found that subjects treated with elenbecestat (E2609), a β-site amyloid precursor protein cleaving enzyme (BACE) inhibitor, who had amyloid baseline positron emission tomography (PET) standard uptake value ratios (SUVr values) of 1.4 to 1.9, exhibited the greatest slowing of cognitive decline while on treatment. See Lynch, S. Y. et al. “Elenbecestat, a BACE inhibitor: results from a Phase 2 study in subjects with mild cognitive impairment and mild-to-moderate dementia due to Alzheimer's disease.” Poster P4-389, Alzheimer's Association International Conference, Jul. 22-26, 2018, Chicago, IL, USA. Similarly, it has been found that subjects having a baseline florbetapir amyloid PET SUVr levels below 1.2 do not exhibit enough cognitive decline to be detectable, whereas subjects having SUVr levels above 1.6 appear to correlate with a plateau effect in which amyloid level has reached a saturation level and treatment does not result in a change of cognitive measures. See Dhadda, S. et al., “Baseline florbetapir amyloid PET standard update value ratio (SUVr) can predict clinical progression in prodromal Alzheimer's disease (pAD).” Poster P4-291, Alzheimer's Association International Conference, Jul. 22-26, 2018, Chicago, IL, USA.

“Early AD” or “early Alzheimer's disease” (EAD), as used herein, is a continuum of AD severity from mild cognitive impairment due to AD—intermediate likelihood to mild Alzheimer's disease dementia. Subjects with early AD include subjects with mild Alzheimer's disease dementia as defined herein and subject with mild cognitive impairment (MCI) due to AD—intermediate likelihood as defined herein. In some embodiments, subjects with early AD have a score of 22-30 on the Mini-Mental State Examination (MMSE) and CDR global range 0.5 to 1.0. Other methods for detecting early AD disease may employ the tests and assays specified below, including the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Other methods include CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, a subject with early AD has evidence of elevated amyloid in the brain or a positive amyloid load. In some embodiments, elevated amyloid in the brain or a positive amyloid load is indicated and/or confirmed by PET assessment. In some embodiments, elevated amyloid in the brain or a positive amyloid load is indicated and/or confirmed by a CSF assessment of markers such as Aβ1-42 (e.g., a soluble CSF biomarker analysis). In some embodiments, elevated amyloid in the brain or a positive amyloid load is indicated and/or confirmed by measuring the concentration of amyloid p1-42 (Aβ42) and a concentration of amyloid β1-40 (Aβ40) and calculating a ratio of Aβ42 to Aβ40 (Aβ42/40 ratio or Aβ1-42/1-40 ratio). In some embodiments, elevated amyloid in the brain or a positive amyloid load is indicated and/or confirmed by an MRI. In some embodiments, elevated amyloid in the brain or a positive amyloid load is indicated by retinal amyloid accumulation. In some embodiments, more than one assessment method is used.

In addition to measuring a serum or plasma Aβ1-42/1-40 ratio in a sample from a subject, the subject's amyloid level may alternatively be detected, or additionally confirmed, by one or more biomarkers such as, but not limited to: (a) amyloid detected by PET scan from either a visual read or semiquantitative thresholds (SUVr or centiloid); (b) cerebrospinal fluid (CSF) Aβ 1-42, and/or Aβ 1-42/1-40 ratio; and/or (c) blood biomarkers (such as plasma Aβ 1-42, tau, total tau (T-tau), and/or P-tau (e.g., P-tau181)). Secondary markers may confirm a primary amyloid determination and include but are not limited to markers of neuronal damage such as neurofilament light peptide (NfL) and markers of neuroinflammation such as glial fibrillary acidic protein (GFAP).

As used herein, subjects having “intact cognition” refer to subjects having a score of greater than 27 on the MMSE after education adjustment and a CDR global equal to 0.

A subject's amyloid level can be detected by biomarkers such as, but not limited to: (a) amyloid detected by PET scan from either a visual read or semiquantitative thresholds (SUVr or centiloid); (c) cerebrospinal fluid (CSF) Aβ1-42, and/or Aβ1-42/1-40 ratio; and/or (d) blood biomarkers (i.e. plasma Aβ1-42, Aβ1-42/Aβ1-40, tau, total tau (T-tau), P-tau, and/or NfL). Secondary markers may confirm a primary amyloid determination and include, but are limited to: (a) tau detected by a PET scan; (b) CSF tau, phosphorylated tau (p-tau), neurofilament light peptide (NfL), and/or neurogranin; (c) other blood biomarkers (i.e. tau, total tau (T-tau), P-tau, and/or NfL).

“Amyloid” refers to fibers that are unbranched, usually extracellular, and found in vivo; in addition, the fibers bind the dye Congo Red and then show green birefringence when viewed between crossed polarizers. Amyloid-forming proteins have been identified and associated with serious diseases, including amyloid-β peptide (Aβ) with Alzheimer's disease (AD), islet amyloid polypeptide (IAPP) with diabetes type 2, and prion protein (PrP) with the spongiform encephalopathies. As used herein, “amyloid,” “brain amyloid,” and “amyloid-β peptide (Aβ)” are used interchangeably.

In some embodiments, the subject has “elevated amyloid” or “intermediate amyloid.” As one of ordinary skill in the art will recognize, amyloid levels from amyloid PET can be reported using the Centiloid method in “centiloid” units (CL). (Klunk W E et al. The Centiloid Project: standardizing quantitative amyloid plaque estimation by PET. Alzheimer's Dement. 2015; 11:1-15 e1-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a person of ordinary skill in the art (POSA). For example, a centiloid value of 32.5 can be used as a threshold value for “elevated amyloid,” and an “intermediate amyloid” level refers to an Aβ amyloid PET in the range of 20-32.5 CL. In another example, a centiloid value of 40 can be used as a threshold value for “elevated amyloid,” and an “intermediate amyloid” level refers to an Aβ amyloid PET in the range of 20-40 CL.

As used herein, “ApoE4-positive” subjects and “ApoE4 carriers” refer to subjects who harbor the e4 variant of the apolipoprotein gene. The e4 variant is one of several major alleles of the apolipoprotein gene. The gene is generally responsible for metabolism of fats. It has been found that carriers of the apolipoprotein e4 show significantly greater rates of amyloid retention when compared to non-carriers. (Drzezga, A. et al, “Effect of APOE genotype on amyloid plaque load and gray matter volume in Alzheimer disease.” Neurology. 2009; 72:1487-94.) In some embodiments, the subject is a heterozygous carrier of the apolipoprotein E ε4 gene allele. In some embodiments, the subject is a homozygous carrier of the apolipoprotein E ε4 gene allele. ApoE4 carriers have a greater response to treatment when administered a composition comprising an anti-Aβ protofibril antibody (i.e. lecanemab) than ApoE4 non-carriers. The terms “ApoE4-negative” and “ApoE4 non-carriers” are used interchangeably.

As used herein, whether an early AD subject is “amyloid-positive” or “amyloid-negative” is determined based on whether or not the subject has a positive amyloid load as indicated by a PET assessment of an amyloid imaging agent uptake into the brain, a CSF assessment of the presence of amyloid pathology using assessments of biomarkers, and/or blood or plasma biomarkers. In some embodiments, a qualitative visual read of PET scans will be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach.

Subjects with “mild Alzheimer's disease dementia,” as used herein, are subjects who meet the NIA-AA core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Also included herein are subjects who have a CDR score of 0.5 to 1.0 and a Memory Box score of 0.5 or greater at screening and baseline and subjects that exhibit change in the score on the Wechsler Memory Scale-Revised Logical Memory subscale II (WMS-R LM II).

Subjects with “MCI due to AD—intermediate likelihood,” as used herein are those identified as such in accordance with the NIA-AA core clinical criteria for mild cognitive impairment due to Alzheimer's disease—intermediate likelihood (see McKhann supra). For example, symptomatic but not demented AD subjects with evidence of brain amyloid pathology making them less heterogeneous and more similar to mild Alzheimer's disease dementia subjects in cognitive and functional decline as measured by the ADCOMS Composite Clinical Score defined herein. Also included are subjects who have a CDR score of 0.5 and a Memory Box score of 0.5 or greater at screening and baseline. Furthermore, subjects who report a history of subjective memory decline with gradual onset and slow progression over the last 1 year before screening, which is corroborated by an informant, are also included herein. Memory decline and/or episodic memory impairment can be assessed in a subject by change in the score on the Wechsler Memory Scale-Revised Logical Memory subscale II (WMS-R LM II).

As used herein, the term “treat” refers to obtaining beneficial or desired results including, but not limited to, therapeutic benefit, by which is meant eradication or amelioration of the underlying condition being treated or of one or more of the physiological symptoms associated therewith.

As used herein, the term “prevent” refers to obtaining beneficial or desired results including, but not limited to, prophylactic benefit. For prophylactic benefit, the formulation may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

As used herein, the term “ARIA” refers to amyloid-related imaging abnormality as evaluated using MRI. In some embodiments, ARIA includes amyloid related imaging abnormality edema/effusion (ARIA-E). In some embodiments, ARIA includes amyloid related imaging abnormality hemorrhage (ARIA-H). In some embodiments, subjects with ARIA experience headache, confusion, and/or seizure and these may be used to identify a subject with ARIA or to indicate further evaluation for ARIA. In some embodiments, ARIA is evaluated at specified intervals during treatment. In some embodiments, ARIA is evaluated when the subject experiences symptoms of ARIA. In some embodiments, maximum serum concentration (Cmax) of anti-Aβ protofibril antibody can be used as a predictor of the risk of ARIA-E. In some embodiments, the use of a subcutaneous formulation may provide a reduced risk of ARIA-E (e.g., due to a lower Cmax) compared to an IV administration.

As used herein, the term “clinical decline” refers to a worsening of one or more clinical symptoms of AD. Methods for measuring clinical decline may employ the tests and assays specified herein. In some embodiments, clinical decline is determined by a worsening of ADCOMS. In some embodiments, clinical decline is determined by a worsening of MMSE. In some embodiments, clinical decline is determined by a worsening of ADAS-Cog. In some embodiments, clinical decline is determined by a worsening of Functional Assessment Questionnaire (FAQ). In some embodiments, clinical decline is determined by a worsening of CDR-SB. In some embodiments, clinical decline is determined by a worsening of Wechsler Memory Scale-IV Logical Memory (subscale) I and/or (subscale) II. In some embodiments, clinical decline is determined by a worsening of CDR score. In some embodiments, clinical decline refers to a worsening in one or more biomarkers of AD or brain measurement (e.g., by PET or MRI), e.g., of brain atrophy and/or amyloid accumulation.

As would be understood by one of ordinary skill in the art, digital, computerized, and/or conventional (e.g., pen and paper) cognitive tests may be used to detect early cognitive changes that may signal mild cognitive impairment and/or a risk for developing dementia, and thus may be used to identify subject in need of treatment as disclosed herein. Such tests, for example, may screen for cognitive impairment, and potentially identify individuals with MCI. Tests may use artificial intelligence to analyze cognitive test results to determine whether a case of mild cognitive impairment will escalate into Alzheimer's within a year. Diagnosing the condition early, before symptoms have begun to appear, may be used to assist physicians identify subjects in need of treatment as disclosed herein sooner, potentially delaying onset or lessening the severity of the neurodegenerative disease.

Provided herein is a method of delaying and/or reducing clinical decline in a subject comprising subcutaneously administering to in a subject in need thereof a suitable dose, such as 400 mg to 1500 mg or 400 mg to 800 mg, of an anti-Aβ protofibril antibody. As used herein, “delaying and/or reducing clinical decline” refers to a change in a score (for example in %) relative to placebo as determined by ADCOMS over a given time period. The reduction and/or delay in clinical decline is determined after, for example, 1 month, 6 months, 12 months, 18 months, and/or 60 months. The clinical decline is reduced or delayed by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, or at least 52% relative to placebo as determined by ADCOMS.

As used herein, “ADCOMS” refers to Alzheimer's Disease Composite Score, a composite clinical score based on an analysis of four ADAS-Cog items (delayed word recall, orientation, word recognition, and word finding difficulty), two MMSE items (orientation to time, and drawing), and all six CDR-SB items (personal care, community affairs, home and hobbies, memory, orientation, and judgment and problem solving), as discussed in the Examples and in Wang, J. et al., “ADCOMS: a composite clinical outcome for prodromal Alzheimer's disease trials.” J. Neurol. Neurosurg. Psychiatry. 2016; 87:993-999. ADCOMS was developed to be particularly sensitive to disease progression during early stages of AD (i.e., preclinical AD or early AD).

In some embodiments, a subject is subcutaneously administered a dose, e.g., from 400 mg to 800 mg or from 400 mg to 1500 mg, such as 720 mg, of an anti-Aβ protofibril antibody, e.g., BAN2401, at a certain frequency, e.g., twice weekly, weekly (QW), bi-weekly (every two weeks or Q2W), or monthly, for a period of time, e.g., for 18 months, or until a certain criteria is reached, and then the subject is optionally administered a maintenance dose of the anti-Aβ protofibril antibody at a certain frequency and for a period of time or until a certain criteria is reached. The dose, frequency, period of time administered, and criteria may or may not be the same as the prior treatment dose, frequency, period of time administered, and/or criteria. In some embodiments, the treatment dose is administered twice weekly, e.g., at 720 mg per dose, and the maintenance dose is administered twice weekly or weekly, e.g., at 720 mg per dose. In some embodiments, more than one first dose and more than one second dose of the anti-Aβ protofibril antibody is administered, wherein the second doses are administered at a lower amount and/or a reduced frequency relative to the first doses. In some embodiments, the criteria can include an increase in the Aβ42/40 ratio observed in a sample (e.g., a plasma sample) relative to the ratio in a sample taken from the subject before treatment or a reduction of amyloid PET SUVr.

As used herein, the term “maintenance dose” refers to a dosage administered to a subject to maintain the desired therapeutic effect. In some embodiments, a subject's maintenance dose is the same as the dose during the treatment period. In some embodiments, the maintenance dose is administered subcutaneously. In some embodiments, the maintenance dose is administered once or multiple times. In some embodiments, the maintenance dose is administered weekly, every two weeks, every 4 weeks, every 6 weeks, every 8 weeks, every 10 weeks, every 12 weeks (every three months or quarterly), every 16 weeks, every 24 weeks (every six months or semi-annually), every 48 weeks, monthly, every 2 months, every 3 months, every 4 months, every 6 months, or every 12 months. In some embodiments, the maintenance dose comprises an anti-Aβ protofibril antibody. In some embodiments, the maintenance dose is 300 mg to 800 mg, 300 mg to 400 mg, 400 mg to 500 mg, 400 mg to 450 mg, 450 mg to 500 mg, 500 mg to 600 mg, 500 mg to 550 mg, 550 mg to 600 mg, 600 mg to 700 mg, 600 mg to 650 mg, 650 mg to 700 mg, 700 mg to 800 mg, 700 mg to 750 mg, or 750 mg to 800 mg. In some embodiments, the maintenance dose is 300 mg, 310 mg, 320 mg, 330 mg, 340 mg, 350 mg, 360 mg, 370 mg, 380 mg, or 390 mg. In some embodiments, the maintenance dose is 400 mg, 410 mg, 420 mg, 430 mg, 440 mg, 450 mg, 460 mg, 470 mg, 480 mg, or 490 mg. In some embodiments, the maintenance dose is 500 mg, 510 mg, 520 mg, 530 mg, 540 mg, 550 mg, 560 mg, 570 mg, 580 mg, or 590 mg. In some embodiments, the maintenance dose is 600 mg, 610 mg, 620 mg, 630 mg, 640 mg, 650 mg, 660 mg, 670 mg, 680 mg, or 690 mg. In some embodiments, the maintenance dose is 700 mg, 710 mg, 720 mg, 730 mg, 740 mg, 750 mg, 760 mg, 770 mg, 780 mg, or 790 mg. In some embodiments, the maintenance dose is 800 mg to 1600 mg, 800 mg to 1000 mg, 800 mg to 900 mg, 900 mg to 1000 mg, 1000 mg to 1200 mg, 1000 mg tov 1100 mg, 1100 mg to 1200 mg, 1200 mg to 1400 mg, 1200 mg to 1300 mg, 1300 mg to 1400 mg, 1400 mg to 1600 mg, 1400 mg to 1500 mg, or 1500 mg to 16000 mg. In some embodiments, the maintenance dose is 800 mg, 820 mg, 840 mg, 860 mg, 880 mg, 900 mg, 920 mg, 940 mg, 960 mg, or 980 mg. In some embodiments, the maintenance dose is 1000 mg, 1020 mg, 1040 mg, 1060 mg, 1080 mg, 1100 mg, 1120 mg, 1140 mg, 1160 mg, or 1180 mg. In some embodiments, the maintenance dose is 1200 mg, 1220 mg, 1240 mg, 1260 mg, 1280 mg, 1300 mg, 1320 mg, 1340 mg, 1360 mg, or 1380 mg. In some embodiments, the maintenance dose is 1400 mg, 1420 mg, 1440 mg, 1460 mg, 1480 mg, 1500 mg, 1520 mg, 1540 mg, 1560 mg, or 1580 mg. In some embodiments, the maintenance dose is provided in a single administration, e.g., administered as a single subcutaneous injection of 1440 mg, or in two or more administrations, two administrations of 720 mg for a total of 1440 mg, four administrations of 360 mg for a total of 1440 mg. In some embodiments, the maintenance dose is 3600 mg. In some embodiments, the maintenance dose is 440 mg. In some embodiments, the maintenance dose is 580 mg. In some embodiments, the maintenance dose is 720 mg. In some embodiments, the maintenance dose of 720 mg is provided in a single administration or in two administrations of 360 mg. In some embodiments, the maintenance dose is 1440 mg. In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg. or four administrations of 360 mg for a total of 1440 mg. In some embodiments, the maintenance dose is 120 mg. In some embodiments, the maintenance dose is 180 mg. In some embodiments, the maintenance dose is 240 mg. In some embodiments, the maintenance dose is 360 mg. In some embodiments, the maintenance dose is 440 mg. In some embodiments, the maintenance dose is 480 mg. In some embodiments, the maintenance dose is 540 mg. In some embodiments, the maintenance dose is 440 mg. In some embodiments, the maintenance dose is 580 mg. In some embodiments, the maintenance dose is 600 mg. In some embodiments, the maintenance dose is 720 mg. In some embodiments, the maintenance dose is 840 mg. In some embodiments, the maintenance dose is 900 mg. In some embodiments, the maintenance dose is 960 mg. In some embodiments, the maintenance dose is 1080 mg. In some embodiments, the maintenance dose is 1200 mg. In some embodiments, the maintenance dose is 1260 mg. In some embodiments, the maintenance dose is 1320 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 1440 mg. In some embodiments, the maintenance dose is provided in a single, biweekly administration of 1440 mg comprising two concurrent, e.g., two sequential administrations of 720 mg of the subcutaneous formulation for a total of 1440 mg or four sequential administrations of 360 mg for a total of 1440 mg.

In some embodiments, the maintenance dose is administered once or multiple times. In some embodiments, the maintenance dose is administered at a lower dose than during an earlier course of treatment and/or is administered less frequently than during the earlier course of treatment.

In some embodiments, the maintenance dose is administered as a subcutaneous injection. In some embodiments, the maintenance dose is administered as a weekly, subcutaneous injection. In some embodiments, the maintenance dose is administered as a biweekly, subcutaneous injection. In some embodiments, the maintenance dose is administered as a monthly, subcutaneous injection. In some embodiments, the maintenance dose is administered as a quarterly, subcutaneous injection.

In some embodiments, the frequency of the maintenance dose is every week. In some embodiments, the maintenance dose is every two weeks (bi-weekly). In some embodiments, the maintenance dose is every four weeks (monthly). In some embodiments, the subcutaneous maintenance dose is administered every six weeks. In some embodiments, the subcutaneous maintenance dose is administered every eight weeks (2 months). In some embodiments, the maintenance dose is every three months (every twelve weeks or quarterly). In some embodiments, the maintenance dose is every six months (every 24 weeks or semi-annually). In some embodiments, a subject's maintenance dose is the same as the dose during the treatment period. In some embodiments, the maintenance dose is same dose amount as the dose prior to administering the maintenance dose. In some embodiments, the maintenance dose amount is lower dose than the dose prior to administering the maintenance dose. In some embodiments, the maintenance dose is same dose frequency as the dose prior to administering the maintenance dose. In some embodiments, the maintenance dose is lower dose frequency than the dose prior to administering the maintenance dose.

In some embodiments, the maintenance dose is administered as a subcutaneous injection of the anti-AP protofibril antibody (e.g., BAN2401). In some embodiments, the maintenance dose is administered as a weekly subcutaneous injection of the subcutaneous formulation of the anti-AP protofibril antibody. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the subcutaneous maintenance dose is administered weekly. In some embodiments, the subcutaneous maintenance dose is administered every two weeks. In some embodiments, the subcutaneous maintenance dose is administered every four weeks (monthly). In some embodiments, the subcutaneous maintenance dose is administered every six weeks. In some embodiments, the subcutaneous maintenance dose is administered every eight weeks (2 months). In some embodiments, the subcutaneous maintenance dose is administered every three months (twelve weeks or quarterly). In some subcutaneous embodiments, the maintenance dose is administered weekly, every two weeks, every 4 weeks, every 6 weeks, every 8 weeks, every 10 weeks, every 12 weeks, every 16 weeks, every 24 weeks, every 48 weeks, monthly, every 2 months, every 3 months, every 4 months, every 6 months, or every 12 months. In some embodiments, the subcutaneous maintenance dose comprises an anti-Aβ protofibril antibody at a dose of 300 mg to 800 mg, 300 mg to 400 mg, 400 mg to 500 mg, 400 mg to 450 mg, 450 mg to 500 mg, 500 mg to 600 mg, 500 mg to 550 mg, 550 mg to 600 mg, 600 mg to 700 mg, 600 mg to 650 mg, 650 mg to 700 mg, 700 mg to 800 mg, 700 mg to 750 mg, or 750 mg to 800 mg. In some embodiments, the maintenance dose is 300 mg, 310 mg, 320 mg, 330 mg, 340 mg, 350 mg, 360 mg, 370 mg, 380 mg, or 390 mg. In some embodiments, the maintenance dose is 400 mg, 410 mg, 420 mg, 430 mg, 440 mg, 450 mg, 460 mg, 470 mg, 480 mg, or 490 mg. In some embodiments, the maintenance dose is 500 mg, 510 mg, 520 mg, 530 mg, 540 mg, 550 mg, 560 mg, 570 mg, 580 mg, or 590 mg. In some embodiments, the maintenance dose is 600 mg, 610 mg, 620 mg, 630 mg, 640 mg, 650 mg, 660 mg, 670 mg, 680 mg, or 690 mg. In some embodiments, the maintenance dose is 700 mg, 710 mg, 720 mg, 730 mg, 740 mg, 750 mg, 760 mg, 770 mg, 780 mg, or 790 mg. In some embodiments, the maintenance dose is 800 mg to 1600 mg, 800 mg to 1000 mg, 800 mg to 900 mg, 900 mg to 1000 mg, 1000 mg to 1200 mg, 1000 mg to 1100 mg, 1100 mg to 1200 mg, 1200 mg to 1400 mg, 1200 mg to 1300 mg, 1300 mg to 1400 mg, 1400 mg to 1600 mg, 1400 mg to 1500 mg, or 1500 mg to 16000 mg. In some embodiments, the maintenance dose is 800 mg, 820 mg, 840 mg, 860 mg, 880 mg, 900 mg, 920 mg, 940 mg, 960 mg, or 980 mg. In some embodiments, the maintenance dose is 1000 mg, 1020 mg, 1040 mg, 1060 mg, 1080 mg, 1100 mg, 1120 mg, 1140 mg, 1160 mg, or 1180 mg. In some embodiments, the maintenance dose is 1200 mg, 1220 mg, 1240 mg, 1260 mg, 1280 mg, 1300 mg, 1320 mg, 1340 mg, 1360 mg, or 1380 mg. In some embodiments, the maintenance dose is 1400 mg, 1420 mg, 1440 mg, 1460 mg, 1480 mg, 1500 mg, 1520 mg, 1540 mg, 1560 mg, or 1580 mg. In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg, or four administrations of 360 mg for a total of 1440 mg. In some embodiments, the maintenance dose is 440 mg. In some embodiments, the maintenance dose is 580 mg. In some embodiments, the maintenance dose is administered as a single administration of 720 mg or two administrations of 360 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 360 mg. In some embodiments, the maintenance dose is administered as a biweekly, subcutaneous injection of 720 mg. In some embodiments, the maintenance dose is administered as a biweekly, subcutaneous injection of 1440 mg. In some embodiments, the maintenance dose is provided in a single, biweekly administration of 1440 mg comprising two concurrent, e.g., sequential administrations of 720 mg of the subcutaneous formulation for a total of 1440 mg.

In some embodiments, a treatment comprises subcutaneously administering an anti-AP protofibril antibody, e.g., BAN2401, before switching to an intravenous maintenance dose. In some embodiments, a treatment comprises subcutaneously administering BAN2401 weekly, e.g., a subcutaneous injection of 720 mg comprising two concurrent, e.g., sequential, injections of 360 mg (2×1.8 mL of 400 mg/2 mL), e.g., until a patient is amyloid-negative or e.g., for at least 18 months. In some embodiments, a treatment comprises subcutaneously administering BAN2401 weekly, e.g., at a dose of 720 mg, e.g., for at least 18 months or e.g., until a patient is amyloid-negative, and then switching to a maintenance dose. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg weekly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg biweekly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg monthly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg every six weeks. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg every eight weeks. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg quarterly. In some embodiments, a subject's maintenance dose is administered at the same amount and/or frequency as the dose during the treatment period. In some embodiments, a subject's maintenance dose is 50% of the dose during the treatment period.

In some embodiments, the maintenance dose is administered intravenously, e.g., after an intravenous treatment period as disclosed above. In some embodiments, an intravenous maintenance dose, e.g., a dosing of 10 mg/kg BAN2401, is administered every week, two weeks, every month, every two months, or every three months (quarterly). In some embodiments, the intravenous maintenance dose is administered every two weeks. In some embodiments, the intravenous maintenance dose is administered every four weeks. In some embodiments, the intravenous maintenance dose is administered every six weeks. In some embodiments, the intravenous maintenance dose is administered every eight weeks (2 months). In some embodiments, the intravenous maintenance dose is administered every three months (quarterly). In some embodiments, the intravenous maintenance dose is administered every 24 weeks (every six months or semi-annually). In some embodiments, the intravenous maintenance dose is 2.5 mg/kg-10 mg/kg. In some embodiments, the maintenance dose is administered as a biweekly, intravenous dose of 10 mg/kg BAN2401. In some embodiments, the maintenance dose is administered as an intravenous dose of 10 mg/kg every four weeks (monthly). In some embodiments, the maintenance dose is administered as an intravenous dose of 10 mg/kg every six weeks. In some embodiments, the maintenance dose is administered as an intravenous dose of 10 mg/kg every eight weeks (2 months). In some embodiments, the maintenance dose is administered as an intravenous dose of 10 mg/kg every twelve weeks (every three months or quarterly). In some embodiments, the maintenance dose is administered as an intravenous dose of 10 mg/kg every 24 weeks (every six months or semi-annually). In some embodiments, a treatment comprises administering intravenously an anti-Aβ protofibril antibody at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every six weeks. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every eight weeks. In some embodiments, a treatment comprises administering intravenously an anti-Aβ protofibril antibody at 10 mg/kg, biweekly, e.g., for at least 18 months or e.g., until a patient is amyloid-negative, before switching to a quarterly intravenous maintenance dose.

In some embodiments, a patient starts on an intravenous maintenance dose, e.g., a dosing of 10 mg/kg BAN2401 as disclosed above before switching to a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation. In some embodiments, a patient starts on a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation before switching to an intravenous maintenance dose, e.g., a dosing of 10 mg/kg BAN2401 as disclosed above.

In some embodiments, a patient is moved back from a maintenance dose to the initial treatment dose if the patient is determined to no longer be amyloid negative, e.g., as assessed by measuring an Aβ42/40 ratio below 0.092 in a blood sample taken after switching to a maintenance dose and/or as determined by PET SUVr. In some embodiments, a patient's treatment is discontinued if the patient is determined to no longer be amyloid negative, e.g., as assessed by measuring an Aβ42/40 ratio below 0.092 in a blood sample taken after switching to a maintenance dose.

In some embodiments, the desired therapeutic effect to be maintained with the maintenance dose may be one or more of a reduction of brain amyloid level, reduction of amyloid PET SUVr, increase of plasma Aβ42/40 ratio, reduction of plasma p-tau181, and changes in other biomarkers correlating with brain amyloid reduction, that achieve sufficient or predetermined levels.

In some embodiments, provided herein is a method of reducing and/or slowing clinical decline in a subject, e.g., one having Pre-AD or early Alzheimer's disease, comprising administering a therapeutically effective amount of at least one anti-Aβ protofibril antibody (e.g., BAN2401) to a patient having an Aβ42/40 ratio less than 0.092. In some embodiments, the anti-Aβ protofibril antibody (e.g., BAN2401) is administered in a therapeutically effective amount to increase the Aβ42/40 ratio above 0.092. In some embodiments, increasing the Aβ42/40 ratio slows the cognitive decline of a patient (e.g., one having pre-AD or early AD) relative to the decline in the absence of treatment.

In some embodiments, the maintenance dose is administered at least every three months (e.g., quarterly) or every twelve weeks. In some embodiments, after switching to a maintenance dose, the Aβ42/40 ratio is measured in a sample (e.g., a plasma sample) from the subject. In some embodiments, the maintenance dose and/or frequency is selected to maintain an Aβ42/40 ratio achieved after the completion of the initial treatment (e.g., after 18 months of treatment). In some embodiments, the maintenance dose and/or frequency is selected to maintain a Aβ42/40 ratio at or above 0.092. In some embodiments, the maintenance dose is continued if the Aβ42/40 ratio remains unchanged or increases. In some embodiments, a patient's amyloid level may be monitored during the treatment with the maintenance dose, e.g., by a blood biomarker. In some embodiments, a patient's amyloid level may be monitored during the treatment with the maintenance dose by one or more biomarkers such as, but not limited to: (a) amyloid detected by PET scan from either a visual read or semiquantitative thresholds (SUVr or centiloid); (b) cerebrospinal fluid (CSF) Aβ1-42, and/or Aβ1-42/1-40 ratio; and/or (c) blood biomarkers (such as plasma Aβ1-42, total tau (T-tau), and/or phosphorylated tau (P-tau)). In some embodiments, a patient's biomarkers may be monitored at least once after switching to the maintenance dose. In some embodiments, a patient's biomarkers are evaluated at least 1 week, 2 weeks, 3 weeks, 1 month, 2 months, 3 months, 6 months, 12 month, 18 months, or 24 months after switching to the maintenance dose In some embodiments, a subject is returned to the original dosing if one or more biomarkers worsen, e.g., if the Aβ42/40 ratio reduces relative the ratio measured in a sample at the end of the treatment period (e.g., at 18 months after the start of treatment). In some embodiments, a subject is administered a higher dose (e.g., a 50% increase in the maintenance dose) if one or more biomarkers worsen, e.g., if the Aβ42/40 ratio reduces relative the ratio measured in a sample at the end of the earlier treatment period (e.g., at 18 months after the start of treatment). In some embodiments, a subject is administered treatment at a higher frequency (e.g., a change from biweekly to weekly administration) if one or more biomarkers worsen, e.g., if the Aβ42/40 ratio reduces relative the ratio measured in a sample at the end of the earlier treatment period (e.g., at 18 months after the start of treatment). In some embodiments, a subject's maintenance dose is the same as the dose during the treatment period. In some embodiments, a maintenance dose is selected (e.g., in conjunction with the evaluation of a change in the Aβ42/40 ratio) based on whether the patient is an ApoE4 carrier, e.g., with a greater increase in the Aβ42/40 ratio required to move from the initial treatment to a maintenance dose for a carrier than for a non-carrier. In some embodiments, after switching to a maintenance dose, the pTau181 level is measured in a sample (e.g., a plasma sample) from the subject. In some embodiments, the maintenance dose and/or frequency is selected to maintain a pTau181 level achieved after the completion of the initial treatment. In some embodiments, the maintenance dose is continued if the pTau181 level remains unchanged. In some embodiments, a subject is returned to the original dosing if the pTau181 level is increased relative the ratio measured in a sample at the end of the treatment period (e.g., at 18 months after the start of treatment). In some embodiments, a maintenance dose is selected (e.g., in conjunction with the evaluation of a change in the pTau181 level) based on whether the patient is an ApoE4 carrier, e.g., with a greater decrease in the pTau181 level required to move to a maintenance dose for a carrier than for a non-carrier. In some embodiments, the maintenance dose comprises two or more dosings, in which a first dosing is selected from the maintenance dose as exemplified above and a second and/or subsequent dosing comprising a lower amount and/or frequency than the first or the previous dosing, respectively. In some embodiments, the switching to the second or subsequent dosing is determined based on one or more biomarkers as exemplified above, where the levels of the biomarkers are different from (e.g., improved over) the levels used in switching from an initial dose to the first dosing in the maintenance dose. In some embodiments, a patient's biomarkers are monitored at least 1 week, 2 weeks, 3 weeks, 1 month, 2 months, 3 months, 6 months, 12 month, 18 months, or 24 months after switching to a maintenance dose. In some embodiments, a patient's biomarkers are monitored every week, every 2 weeks, every 3 weeks, monthly, every 2 months, every 3 months, every 6 months, every 12 month (every year), every 18 months (every 1.5 months), or every 24 months (every 2 years) after switching to a maintenance dose.

In some embodiments, after switching to a maintenance dose, a subject's biomarker levels will indicate increasing levels of amyloid in the brain. In some embodiments, after switching to a maintenance dose, a subject's biomarker levels, e.g. the plasma Aβ42/40 ratio, will began to decrease, indicating increasing levels of amyloid in the brain. In some embodiments, a subject on a maintenance dose will have a decrease in the Aβ42/40 ratio. In some embodiments, a subject is put on a maintenance dose chosen such that the subject will have a decrease in the Aβ42/40 ratio but the Aβ42/40 ratio will remain below the threshold for amyloid positivity, e.g. for at least one year (e.g., at least 1, 2, 3, 4, 5, 6, 7, 8, 9, or 10 years).

In some embodiments, after switching to a maintenance dose, a subject's biomarker levels, e.g. p-tau181, will began to increase, indicating increasing levels of amyloid in the brain. In some embodiments, a subject on a maintenance dose will have an increase in plasma p-tau181. In some embodiments, a subject on a maintenance dose will have an increase in p-tau181 but the level p-tau181 will remain above the threshold for amyloid positivity, e.g., for at least one year (e.g., at least 1, 2, 3, 4, 5, 6, 7, 8, 9, or 10 years).

In some embodiments, a patient's treatment is discontinued if a patient no longer has early AD, e.g., as assessed by cognitive evaluation, PET SUVr, and/or plasma biomarkers such as an Aβ42/40 ratio (e.g., if an Aβ42/40 ratio drops below 0.092 or an SUVr negativity increases above 1.17 as measured using florbetapir).

In some embodiments, the maintenance dose and/or frequency is selected to maintain a PET SUVr negativity level achieved after the completion of the initial treatment, e.g., a level of 1.17 as measured using florbetapir. In some embodiments, after switching to a maintenance dose, a PET SUVr level is measured. In some embodiments, the maintenance dose and/or frequency is selected to maintain a PET SUVr level achieved after the completion of the initial treatment. In some embodiments, the maintenance dose is continued if the PET SUVr level remains unchanged. In some embodiments, a subject is returned to the original dosing if the PET SUVr level is increased relative the ratio measured in a sample at the end of the treatment period (e.g., at 18 months after the start of treatment). In some embodiments, a maintenance dose is selected (e.g., in conjunction with the evaluation of a change in the PET SUVr) based on whether the patient is an ApoE4 carrier, e.g., with a greater decrease in the PET SUVr level required to move to a maintenance dose for a carrier than for a non-carrier.

In some embodiments, a treatment is discontinued if a favorable biomarker level is achieved. In some embodiments, a treatment is discontinued if a favorable biomarker level is achieved after the completion of the initial treatment. In some embodiments, a treatment is discontinued if a favorable biomarker level is achieved and/or maintained (e.g., for a set period of time such as six months or a year) during a maintenance dosing. In some embodiments, a treatment is discontinued if a high Aβ42/40 ratio (e.g., an Aβ42/40 ratio at 0.09, 0.091, 0.092, 0.093, 0.094, 0.095, 0.096, 0.097, 0.099, 0.1) is achieved, e.g., after the completion of the initial treatment or during a maintenance dosing regimen. In some embodiments, a treatment is discontinued if an Aβ42/40 ratio at or above 0.092 is achieved. In some embodiments, a treatment is discontinued if an Aβ42/40 ratio above 0.092 is achieved. In some embodiments, a treatment is discontinued if an SUVr amyloid negativity level is at or below 1.17 as measured using florbetapir after the completion of the initial treatment or during a maintenance dosing regimen.

In some embodiments, a maintenance dose is discontinued if a favorable biomarker level is achieved after the completion of a set period of time on the maintenance treatment (e.g., six months or a year). In some embodiments, a maintenance dose is discontinued if a high Aβ42/40 ratio (e.g., an Aβ42/40 ratio at 0.09, 0.091, 0.092, 0.093, 0.094, 0.095, 0.096, 0.097, 0.099, 0.1) is achieved. In some embodiments, a maintenance dose is discontinued if an Aβ42/40 ratio at or above 0.092 is achieved. In some embodiments, a treatment is discontinued if an Aβ42/40 ratio above 0.092 is achieved. In some embodiments, a maintenance dose is discontinued if the SUVr amyloid negativity level is at or below 1.17 as measured using florbetapir.

In some embodiments, a maintenance dose is discontinued if a favorable biomarker level is not maintained over the course of a maintenance treatment (e.g., if an Aβ42/40 ratio drops below 0.092 or an SUVr negativity increases above 1.17 as measured using florbetapir). In some embodiments, a maintenance dose is discontinued if a favorable biomarker level is not maintained over the course of a maintenance treatment (e.g., if an Aβ42/40 ratio drops below 0.092 and/or an SUVr negativity increases above 1.17 as measured using florbetapir).

In some embodiments, a patient's amyloid level may be monitored for regression after treatment discontinuation, e.g., by a blood biomarker. In some embodiments, a patient's amyloid level may be monitored for regression after treatment discontinuation by one or more biomarkers such as, but not limited to: (a) amyloid detected by PET scan from either a visual read or semiquantitative thresholds (SUVr or centiloid); (b) cerebrospinal fluid (CSF) Aβ1-42, and/or Aβ1-42/1-40 ratio; and/or (c) blood biomarkers (such as plasma Aβ1-42, tau, total tau (T-tau), and/or P-tau (e.g. pTau181)). In some embodiments, a patient's biomarkers may be monitored at least once after the discontinuation of treatment. In some embodiments, a patient's biomarkers are monitored at least 1 week, 2 weeks, 3 weeks, 1 month, 2 months, 3 months, 6 months, 12 month, 18 months, or 24 months after treatment discontinuation. In some embodiments, treatment is reinitiated if a patient's biomarker level becomes less favorable, e.g., a reduction in an Aβ42/40 ratio, e.g., to less than 0.092. In some embodiments, treatment is reinitiated if the amyloid level is found to regress after treatment discontinuation. In some embodiments, treatment is reinitiated if a patient's biomarker level becomes less favorable, e.g., a reduction in an Aβ42/40 ratio, e.g., to less than 0.092.

In some embodiments, the maintenance dose is administered at least every three months (e.g., every three months, every two months, or monthly). In some embodiments, the maintenance dose is administered at least every month. In some embodiments, the maintenance dose and/or frequency is selected to maintain a PET SUVr level achieved after the completion of the initial treatment. In some embodiments, the maintenance dose is selected to maintain a PET SUVr level at or below amyloid negativity (e.g. for florbetapir, PET SUVr of 1.17).

In some embodiments, a subject is administered a dose of the anti-Aβ protofibril antibody without an initial titrating step up to the treatment dose. In some embodiments, a dose of lecanemab may be used in treating AD without the need of a prior titrating step.

As used herein, the term “PET” or “Amyloid PET” refers to Amyloid positron emission tomography imaging. In some embodiments, PET imaging (also referred to as a PET scan) is performed to assess for amyloid pathology. In some embodiments, amyloid PET is assessed with a PET tracer and uses the same tracer in follow-up assessments. In some embodiments, the PET imaging uses a florbetapir tracer. In some embodiments, the PET imaging uses a flutemetamol tracer.

Amyloid positron emission tomography (PET) imaging can be used to confirm the presence of amyloid pathology in the brain of early AD subjects in the screening phase of the study and/or to evaluate the effects of the at least one anti-AB antibody on amyloid levels in the brain, both by whole brain analysis (e.g., the average of 5-6 cortical regions) and brain region analysis. In some embodiments, the PET scan uses florbetapir. In some embodiments, amyloid plaque load can be identified by a PET imaging uptake visual read, e.g., by a trained radiologist. In some embodiments, 2 readers (1 designated as Primary Reader) visually assess the images to determine whether the scan is positive or negative for amyloid. In further embodiments, four regions of the brain are assessed for uptake of the imaging agent: the temporal lobes, the occipital lobes, the prefrontal cortex, and the parietal cortex and a positive amyloid scan has either 1 region with intense gray matter uptake that is greater than the white matter uptake and extends to the outer edges of the brain, or 2 regions with areas of reduced gray-white contrast. In further embodiments, if disagreement occurs between 2 readers, both meet to review the scan for a consensus read.

In some embodiments, amyloid plaque load can be identified by a standard uptake value ratio (SUVr) as compared to a reference region. Methods for calculating PET SUVr are known in the art and may include those described herein. In some embodiments, a Standard Uptake Value Ratio Quantitative analysis of amyloid levels is completed using PMOD Biomedical Image Quantification Software (PMOD Technologies, Zurich, Switzerland). In some embodiments, PET images are first assessed for subject movement in the X, Y, and Z planes and corrected for motion, if needed, before individual images (e.g., 5-minute emission frames) are averaged, e.g., using a PMOD Averaging Function (PET frames averaged to increase the signal to noise ratio). In some embodiments, corresponding MRIs from subjects are prepared (e.g., using matrix size reduction processing, cropping of the MRI to include only the brain, segmentation to separate images into binary maps of gray matter, white matter, and CSF, and stripping the image of skull leaving only brain mask). In some embodiments, the averaged PET images and prepared MRIs are matched using the PMOD Matching Function, placing the images in the same orientation. In some embodiments, a Brain Normalization function, e.g., as provided by PMOD software, is used along with Brain Norm and Rigid Matching transformation matrices, to produce an averaged PET. In some embodiments, this averaged PET which is normalized to the MNInst space (Senjem et al, 2005) that is in the same orientation as the subject's segmented MRI for quantitative analysis. In some embodiments, the PMOD Mask Function is used to mask the brain and zero the image outside of the mask to create a Normalized Gray Matter PET and a Normalized White Matter PET. Standard uptake values (SUVs) may be calculated for all gray matter mapped regions and the 3 white matter regions (pons, cerebellar white, and subcortical white) using PMOD software calculated using the normalized PET, subject weight, and injected dose of tracer to arrive at the units of SUVs. In some embodiments, the SUVr is the ratio of the global cortical average as compared to a reference region of choice. In some embodiments, a whole cerebellum mask is used as the reference region. In some embodiments, the reference region is subcortical white matter, derived whole cerebellum, whole cerebellum adjusted by subcortical white matter, cerebellar gray matter, and composite reference regions consisting of cerebellar cortex, pons subcortical white matter, and cerebella white matter.

In some embodiments, after administration of the first dose of the composition the adjusted mean change from baseline in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to baseline. In some embodiments, the adjusted mean change from baseline in a subject's PET SUVr value is reduced by −0.20 to −0.30.

In some embodiments, the efficacy of the treatment for Alzheimer's Disease can be measured by, for example, any one or a combination of medical observations, cognitive assessments, medical diagnostic, and medical imaging such as: prevention of brain amyloid accumulation by amyloid PET at 216 weeks, delay of tau PET accumulation; change from baseline in amyloid PET standard uptake value ratio (SUVr) at week 216; change from baseline in tau PET SUVr at week 216; change in the Preclinical Alzheimer's Disease Cognitive Composite 5 (PACC5) scale; change in levels of complement C3; change in the score on the Wechsler Memory Scale-Revised Logical Memory subscale II (WMS-R LM II); a change in the score on the Cogstate International Shopping List Test (ISLT); change in score on the Trail Making Test (TMT; change in score on the Cognitive Function Instrument (CFI); change in score on the Alzheimer's Disease Cooperative Study-Activities of Daily Living Scale (ADCS-ADL); change in score on the Clinical Dementia Rating Scale Sum of Boxes (CDR-SB); volumetric magnetic resonance imaging (vMRI); resting state functional magnetic resonance imaging (rs-fMRI); change in levels of biomarkers in cerebrospinal fluid, such as: Aβ[1-42], Aβ[1-40], t-tau, p-tau, neurogranin, and neurofilament light chain protein (NfL); change in levels of biomarkers in plasma and/or blood; and/or time to amyloid negativity threshold.

In some embodiments, the efficacy of the treatment for preclinical Alzheimer's Disease can be measured by, for example, any one or a combination of medical observations, cognitive assessments, medical diagnostic, and medical imaging such as: change from baseline in Preclinical Alzheimer's Disease Cognitive Composite 5 (PACC5) scale at 216 weeks; change from baseline in amyloid PET SUVr at weeks 96 and 216; change from baseline in tau PET SUVr at weeks 96 and 216; change from baseline in Cognitive Function Index (CFI) at week 216; change in levels of complement C3; change in score on the Cogstate International Shopping List Test (ISLT); change in score on the Trail Making Test (TMT; change in score on the Cognitive Function Instrument (CFI); change in score on the Alzheimer's Disease Cooperative Study-Activities of Daily Living Scale (ADCS-ADL); change in score on the Clinical Dementia Rating Scale Sum of Boxes (CDR-SB); volumetric magnetic resonance imaging (vMRI); resting state functional magnetic resonance imaging (rs-fMRI); change in levels of biomarkers in cerebrospinal fluid, such as: Aβ[1-42], Aβ[1-40], t-tau, p-tau, neurogranin, and neurofilament light chain protein (NfL); change in levels of biomarkers in plasma and/or blood; change in time to score to 0.5 on Clinical Dementia Rating scale; and/or change in time to score of 1.17 or lower on standardized uptake value ratio across the whole cerebral cortex (SUVr WC).

In some embodiments, the efficacy of the treatment for Early Alzheimer's Disease can be measured by, for example, any one or a combination of medical observations, cognitive assessments, medical diagnostic, and medical imaging such as: change from baseline in amyloid PET SUVr at months 3, 6, 12, and 18; change from baseline in tau PET SUVr at months 13 and 18; change in levels of biomarkers in cerebrospinal fluid, such as: Aβ[1-42], Aβ[1-40], t-tau, p-tau, neurogranin, and neurofilament light chain protein (NfL); change in score on the Alzheimer's Disease Composite Score (ADCOMS) over 18 months; change in score on the Alzheimer Disease Assessment Scale-Cognitive subscale (ADAS-cog) over 18 months; change in score on the Clinical Dementia Rating Scale Sum of Boxes (CDR-SB); a change in score on the Mini-Mental State Examination (MMSE); change in levels of biomarkers in plasma and/or blood; change in score on the Alzheimer's Disease Cooperative Study-Activities of Daily Living Scale (ADCS-ADL); a change in the grade on the European Quality of Life-5 Dimensions (EQ-5D); a change in the rating on the Quality of Life in Alzheimer's Disease (QOL-AD) scale.

As noted above, disclosed herein are methods for treating and/or preventing Alzheimer's disease comprising subcutaneously administering to a subject in need thereof an anti-Aβ protofibril antibody. Also provided herein are methods of reducing clinical decline in a subject having early Alzheimer's disease, methods of reducing brain amyloid level in a subject, and methods of converting a subject from amyloid positive to amyloid negative comprising subcutaneously administering to a subject in need thereof an anti-Aβ protofibril antibody. In some embodiments, the anti-Aβ protofibril antibody comprises a heavy chain variable regions comprising an amino acid sequence of SEQ ID NO: 1, and a light chain variable region comprising an amino acid sequence of SEQ ID NO: 2.

In some embodiments, the anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

As used herein, a “fragment” of an antibody comprises a portion of the antibody, for example comprising an antigen-binding or a variable region thereof. Non-limiting examples of fragments include Fab fragments, Fab′ fragments, F(ab′)2 fragments, Fv fragments, diabodies, linear antibodies, and single-chain antibody molecules.

The assignment of amino acids to each domain is, generally, in accordance with the definitions of SEQUENCES OF PROTEINS OF IMMUNOLOGICAL INTEREST (Kabat et al., 5th ed., U.S. Department of Health and Human Services, NIH Publication No. 91-3242, 1991, hereafter referred to as “Kabat report”).

In some embodiments, the anti-Aβ protofibril antibody comprises a human constant region. In some embodiments, the human constant region of the anti-Aβ protofibril antibody comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region is chosen from IgG1 and allelic variations thereof. The amino acid sequence of human IgG1 constant region is known in the art and set out in SEQ ID NO: 3.

In some embodiments, the human constant region of the at least one anti-Aβ protofibril antibody comprises a light chain constant region chosen from κ-λ-chain constant regions and any allelic variation thereof as discussed in the Kabat report. Any one or more of such sequences may be used in the present disclosure. In some embodiments, the light chain constant region is chosen from K and allelic variations thereof. The amino acid sequence of human K chain constant region is known in the art and set out in SEQ ID NO: 4.

In some embodiments, the anti-Aβ protofibril antibody comprises a human IgG1 heavy chain constant region, and a human Ig kappa light chain constant region. In some embodiments, the anti-Aβ protofibril antibody comprises a heavy chain constant region comprising an amino acid sequence of SEQ ID NO: 3, and a light chain constant region comprising an amino acid sequence of SEQ ID NO: 4.

In some embodiments, the anti-Aβ protofibril antibody is lecanemab, which is also known as BAN2401. Lecanemab is a humanized IgG1 monoclonal version of mAb158, which is a murine monoclonal antibody raised to target protofibrils and disclosed in WO 2007/108756 and Journal of Alzheimer's Disease 43: 575-588 (2015). Lecanemab is an anti-Aβ protofibril antibody, demonstrating low affinity for Aβ monomer while binding with high selectivity to soluble A3 aggregate species. For example, lecanemab has been reported demonstrates an approximately 1000-fold and 5-fold to 10-fold higher selectivity for soluble Aβ protofibrils than for Aβ monomers or Aβ-insoluble fibrils, respectively.

Lecanemab comprises (i) a heavy chain variable domain comprising the amino acid sequence of SEQ ID NO: 1 and (ii) a light chain variable domain comprising the amino acid sequence of SEQ ID NO: 2. The full length sequence of lecanemab is set forth in SEQ ID NO: 13 and is described in WO 2007/108756 and in Journal of Alzheimer's Disease 43:575-588 (2015).

Other non-limiting examples of suitable antibodies for use as the anti-Aβ protofibril antibody in the present disclosure include those disclosed in WO 2002/003911, WO 2005/123775, WO 2007/108756, WO 2011/001366, WO 2011/104696, and WO 2016/005466.

In some embodiments, the anti-Aβ protofibril antibody is administered subcutaneously (SC). In some embodiments, the anti-Aβ protofibril antibody is administered in an injection having a volume of 1.1 mL. In some embodiments, the anti-Aβ protofibril antibody is administered in an injection having a volume of 1.4 mL. In some embodiments, the anti-Aβ protofibril antibody is administered in an injection having a volume of 1.45 mL. In some embodiments, the anti-Aβ protofibril antibody is administered in an injection having a volume of 1.8 mL.

In some embodiments, the anti-Aβ protofibril antibody is administered once daily. In some embodiments, the anti-Aβ protofibril antibody is administered twice daily. In some embodiments, the anti-Aβ protofibril antibody is administered once or multiple times; for example, the anti-Aβ protofibril antibody is administered as a single an administration of 720 mg or as two administrations of 720 mg for a total of 1440 mg. In some embodiments, the anti-Aβ protofibril antibody is administered weekly. In some embodiments, the anti-Aβ protofibril antibody is administered twice weekly. In some embodiments, the anti-Aβ protofibril antibody is administered three times weekly. In some embodiments, the anti-Aβ protofibril antibody is administered every 2 weeks. In some embodiments, the anti-Aβ protofibril antibody is administered monthly. In some embodiments, the dose amount and/or the dose frequency may be reduced after the desired therapeutic effect is achieved. The reduced frequency may be every two weeks, or every 4 weeks, every 6 weeks, every 8 weeks, every 10 weeks, every 12 weeks, every 16 weeks, monthly, every 2 months, every 3 months, every 4 months, every 6 months, or every 12 months. In some embodiments, the desired therapeutic effect that related to the reduction of the dose amount or the dose frequency may be one or more selected from reduction of brain amyloid, reduction of amyloid PET SUVr, increase of plasma Aβ42/40 ratio, reduction of plasma p-tau181, and changes in other biomarkers correlating with brain amyloid reduction, that achieve sufficient or predetermined levels. In some embodiments, the administration of the anti-Aβ protofibril antibody is discontinued when the desired therapeutic effect is maintained after the reduction of the dose amount or the dose frequency. In some embodiments, the administration of the anti-Aβ protofibril antibody is discontinued if the desired therapeutic effect, which may be evaluated by one or more of selected from reduction of brain amyloid, reduction of amyloid PET SUVr, increase of plasma Aβ42/40 ratio, reduction of plasma p-tau181, and changes in other biomarkers correlating with brain amyloid reduction, is not achieved or expected sufficient or predetermined levels in a subject.

In some embodiments, the methods comprise measuring an Aβ42/40 ratio in a sample, e.g., a blood sample, from a subject having or suspected of having AD before treatment and again in another sample during treatment (although it is to be understood that additional doses may be administered in between the sampling time points). In some embodiments, treatment may be stopped and/or reduced (e.g., reduced frequency and/or dosage) if an increase in the Aβ42/40 ratio is detected between the first and second samplings. In some embodiments, after treatment has been stopped or reduced, a further measurement of the Aβ42/40 ratio may be made in a sample from the subject. In some embodiments, treatment is restarted, dosage is increased, and/or the frequency of administration is increased if a reduction in the Aβ42/40 ratio is detected. In some embodiments, the dosage or frequency of treatment is increased to return to the dosage and/or frequency used in a prior treatment, e.g., before a dose reduction and/or lengthening of the dose frequency had commenced. In some embodiments, the methods comprise measuring an Aβ42/40 ratio in a sample from a subject during treatment and again after stopping treatment or after the dosage or frequency of treatment has been reduced (it is to be understood that additional doses may be administered in between the sampling time points). In some embodiments, if a reduction in the Aβ42/40 ratio is detected, treatment is resumed, or the dosage or frequency of treatment is increased, in comparison to the dose or frequency during the period in which the ratio decreased. In some embodiments, multiple measurements may be made during a treatment prior to a decision to stop treatment and/or reduce treatment based on an elevated Aβ42/40 ratio (e.g., based on a trend showing increase in the Aβ42/40 ratio at each subsequent measurement). In some embodiments, multiple measurements may be taken after treatment has stopped or been reduced, and a decision to resume treatment and/or increase treatment may be taken based on a reduction in Aβ42/40 ratio (e.g., based on a trend showing a reduction in the Aβ42/40 ratio at each subsequent measurement). In some embodiments, following the resumption of treatment or the increased treatment regimen, one or more additional measurements may be made of the Aβ42/40 ratio in a sample from a subject. In some embodiments, treatment is continued if an increase in the Aβ42/40 ratio is observed in the subsequent measurements. In some embodiments, the measurement of the Aβ42/40 is done in conjunction with measuring one or more additional biomarkers (e.g., using a reduction in PET SUVr as an indicator of amyloid plaque reduction during and/or after treatment). In some embodiments, treatment may be stopped if a decrease in the Aβ42/40 ratio is detected between the first and a subsequent, e.g., second, third, or fourth, sampling. In some embodiments, treatment may be stopped due to a low therapeutic effect.

In some embodiments, any of the methods may further comprise measuring one or more additional biomarkers, e.g., measuring phosphorylated tau (P-tau)(e.g., P-tau181). In some embodiments, the measurement of P-tau (e.g., P-tau181) is done in a sample, e.g., a blood sample, from a subject having or suspected of having AD before treatment and again in another sample during treatment (although it is to be understood that additional doses may be administered in between the sampling time points). In some embodiments, treatment may be stopped and/or reduced (e.g., reduced frequency and/or dosage) if a decrease in P-tau181 is detected between the first and second samplings. In some embodiments, after treatment has been stopped or reduced, a further measurement of the P-tau181 may be made in a sample from the subject. In some embodiments, treatment is restarted, dosage is increased, and/or the frequency of administration is increased if an increase in the P-tau181 is detected. In some embodiments, the dosage or frequency of treatment is increased to return to the dosage and/or frequency used in a prior treatment, e.g., before a dose reduction and/or lengthening of the dose frequency had commenced. In some embodiments, the methods comprise measuring P-tau181 in a sample from a subject during treatment and again after stopping treatment or after the dosage or frequency of treatment has been reduced (it is to be understood that additional doses may be administered in between the sampling time points). In some embodiments, if an increase in P-tau181 is detected, treatment is resumed, or the dosage or frequency of treatment is increased, in comparison to the dose or frequency during the period in which the level of P-tau181 decreased. In some embodiments, multiple measurements may be made during a treatment prior to stopping treatment and/or reducing treatment based on a decrease in P-tau181 (e.g., based on a trend showing a decrease in P-tau181 at each subsequent measurement). In some embodiments, multiple measurements may be taken after treatment has stopped or been reduced, before resuming treatment and/or increasing treatment based on an increase in P-tau181 (e.g., based on a trend showing an increase in P-tau181 at each subsequent measurement). In some embodiments, following the resumption of treatment or the increased treatment regimen, one or more additional measurements may be made of P-tau181 in a sample from a subject. In some embodiments, treatment is continued if a decrease in P-tau181 is observed in the subsequent measurements. In some embodiments, the measurement of P-tau181 is done in conjunction with measuring one or more additional biomarkers (e.g., using an increase in the Aβ42/40 ratio an indicator of amyloid plaque reduction during and/or after treatment).

In some embodiments, treatment is stopped and/or reduced (e.g., reduced frequency and/or dosage) if a decrease in P-tau (e.g., P-tau181) is detected between a first and second samplings in a subject and an increase in an Aβ42/40 ratio is detected in the samples. In some embodiments, treatment is resumed and/or increased (e.g., increased frequency and/or dosage) if an increase in P-tau (e.g., P-tau181) is detected after stopping and/or reducing an initial treatment in a subject and a decrease in an Aβ42/40 ratio is detected.

In some embodiments, treatment may be stopped if an increase in P-tau181 is detected between the first and a subsequent, e.g., second, third, or fourth, sampling. In some embodiments, treatment may be stopped due to a low therapeutic effect.

In some embodiments, a treatment comprises subcutaneously administering BAN2401 weekly, e.g., at a dose of 720 mg, e.g., for at least 18 months. In some embodiments, a treatment comprises administering subcutaneously BAN2401 twice weekly, e.g., at 720 mg per dose, e.g., for at least 18 months. In some embodiments, treatment is continued until a desired improvement in one or more biomarker or other treatment outcome measure is achieved, e.g., when an increase in the Aβ42/40 ratio is observed in a sample (e.g., a plasma sample) relative to the ratio in a sample taken from the subject before treatment, e.g., before 18 months of treatment. In some embodiments, the subject has been diagnosed with early AD. In some embodiments, the subject has been diagnosed as having mild cognitive impairment due to Alzheimer's disease—intermediate likelihood and/or has been diagnosed as having mild Alzheimer's disease dementia.

In some embodiments, the method of treatment comprises measuring the concentration of amyloid P 1-42 (Aβ 42) and a concentration of amyloid $1-40 (Aβ 40) in a first blood sample obtained from the subject to determine a first ratio of Aβ42 to Aβ40 (Aβ42/40 ratio). In some embodiments, the subject is then administered a therapeutically effective dose of an anti-amyloid P (A3) protofibril antibody. In some embodiments, a second blood sample is obtained after the first sample to determine a second Aβ42/40 ratio. In some embodiments, a second blood sample is obtained from a subject after treatment has stopped or been reduced. In some embodiments, a change in the Aβ42/40 ratio is used to determine a second therapeutically effective dose. In some embodiments, a subject having an elevated second ratio relative to the first ratio is administered a second therapeutically effective dose comprising the same or a lower amount of the anti-Aβ protofibril antibody than in the first dose to the subject. In some embodiments, a subject having a lower second ratio relative to the first ratio is administered a second therapeutically effective dose comprising a higher amount of the anti-Aβ protofibril antibody than in the first dose. In some embodiments, a subject having a lower second ratio relative to the first ratio is administered a different treatment for AD. A first therapeutically effective dose may be administered multiple times (e.g., biweekly or monthly for 6-18 months) before changing to a second therapeutically effective dose or dosing regimen after measuring a second Aβ42/40 ratio. In some embodiments, a first therapeutically effective dose may be administered for at least 18 months before switching to a maintenance dose. In some embodiments, a first therapeutically effective dose may be administered until a patient is amyloid negative before switching to a maintenance dose. In some embodiments, a first therapeutically effective dose may be administered until a patient is amyloid negative (e.g., as measured by amyloid or tau positron emission tomography (PET), cerebrospinal fluid level of Aβ1-42 and/or Aβ1-42/1-40 ratio, cerebrospinal fluid level of total tau, cerebrospinal fluid level of neurogranin, cerebrospinal fluid level of neurofilament light peptide (NfL), and blood biomarkers as measured in the serum or plasma (e.g. levels of Aβ1-42, the ratio of two forms of amyloid-β peptide (Aβ1-42/1-40 ratio), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and/or neurofilament light (NfL)) before switching to a maintenance dose. In some embodiments, a first therapeutically effective dose may be administered until a patient is amyloid negative, e.g., as measured by an Aβ42/40 ratio at or above 0.092-0.094 (e.g., at or above 0.092) or a florbetapir amyloid PET SUVr negativity at or below 1.17, before switching to a maintenance dose. In some embodiments, a first therapeutically effective dose may be administered until a patient is amyloid negative, e.g., as measured by an Aβ42/40 ratio above 0.092 or a florbetapir amyloid PET SUVr negativity at or below 1.17, before switching to a maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a maintenance dose.

In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose (e.g., at 10 mg/kg, e.g., biweekly, or every 4, 6, 8, 10, or 12 weeks). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a biweekly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a monthly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose every six weeks. In some embodiments, a first therapeutically effective dose comprises administering intravenously an anti-AP protofibril antibody at 10 mg/kg (e.g., administering BAN2401 at 10 mg/kg), biweekly, e.g., for at least 18 months or e.g., until a patient is amyloid-negative before switching to an intravenous maintenance dose every eight weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose every two months. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a quarterly intravenous maintenance dose.

In some embodiments, a first therapeutically effective dose comprises subcutaneously administering an anti-Aβ protofibril antibody at 720 mg (e.g., administering BAN2401 at 720 mg) weekly, e.g., for at least 18 months or e.g., until a patient is amyloid-negative before switching to a subcutaneous maintenance dose (e.g., at 720 mg, e.g., weekly, biweekly, or every 4, 6, 8, 10, or 12 weeks). In some embodiments, the maintenance dose is 360 mg weekly.

In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a weekly subcutaneous maintenance dose (e.g., at a dose of 720 mg). In some embodiments, a first therapeutically effective dose comprises administering intravenously an anti-AP protofibril antibody at 10 mg/kg (e.g., administering BAN2401 at 10 mg/kg), biweekly, e.g., for at least 18 months or e.g., until a patient is amyloid-negative before switching to a weekly subcutaneous maintenance dose (e.g., at a dose of 360 mg). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a biweekly subcutaneous maintenance dose (e.g., at a dose of 720 mg or at a dose of 360 mg). In some embodiments, a first therapeutically effective dose comprises administering intravenously an anti-AP protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every month. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every six weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every eight weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every two months. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a quarterly subcutaneous maintenance dose (e.g., at a dose of 720 mg or at a dose of 360 mg).

In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., at a dose of 720 mg or at a dose of 360 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a biweekly subcutaneous maintenance dose (e.g., at a dose of 720 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., a single dose of 360 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a monthly subcutaneous maintenance dose (e.g., at a dose of 720 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every six weeks. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every eight weeks. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every two months. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a quarterly subcutaneous maintenance dose (e.g., at a dose of 720 mg).

In some embodiments, a treatment comprises administering intravenously an anti-Aβ protofibril antibody at 10 mg/kg (e.g., administering BAN2401 at 10 mg/kg), biweekly, e.g., for at least 18 months. In some embodiments, a treatment comprises administering intravenously an anti-Aβ protofibril antibody before switching to a maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months before switching to a maintenance dose. In some embodiments, a subject is switched to a maintenance dose without an initial titrating step to the maintenance dose. In some embodiments, a subject is switched to a maintenance dose with at least one titrating step to the maintenance dose, e.g., the subject's dosage or frequency of administration may be reduced in multiple steps until achieving a final maintenance dosing regime (e.g., a stepwise reduction from a subcutaneous treatment dosing regimen of 720 mg weekly to a maintenance dosing regimen of 360 mg weekly or 720 mg biweekly via intermediate dosing at intermediate amounts or time periods such as 540 mg weekly or 720 mg every 10 days). In some embodiments, a subject's maintenance dose is the same as the dose during the treatment period. In some embodiments, a subject's maintenance dose is 50% of the dose during the treatment period.

In some embodiments, a treatment comprises subcutaneously administering an anti-Aβ protofibril antibody, e.g., BAN2401, before switching to a subcutaneous maintenance dose. In some embodiments, a treatment comprises subcutaneously administering BAN2401 weekly, e.g., at a dose of 720 mg, e.g., until a patient is amyloid-negative or e.g., for at least 18 months. In some embodiments, a treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., for at least 18 months or e.g., until a patient is amyloid-negative, and then switching to a maintenance dose. In some embodiments, a treatment comprises subcutaneously administering BAN2401 weekly, e.g., at a dose of 720 mg, e.g., for at least 18 months or e.g., until a patient is amyloid-negative, before switching to a weekly, subcutaneous maintenance dose, e.g., a dose of 360 mg. In some embodiments, a treatment comprises subcutaneously administering BAN2401 weekly, e.g., at a dose of 720 mg, e.g., for at least 18 months or e.g., until a patient is amyloid-negative, before switching to a monthly subcutaneous maintenance dose, e.g., a dose of 720 mg. In some embodiments, a subject's maintenance dose is the administered at the same amount and/or frequency as the dose during the treatment period.

In some embodiments, a treatment comprises administering intravenously an anti-Aβ protofibril antibody at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative before switching to a quarterly intravenous maintenance dose.

In some embodiments, a maintenance dose is administered subcutaneously (e.g., as a subcutaneous injection). In other embodiments, a treatment comprises subcutaneously administering an anti-Aβ protofibril antibody before switching to an intravenous maintenance dose. In some embodiments, a treatment comprises administering intravenously an anti-Aβ protofibril antibody before switching to a subcutaneous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a weekly subcutaneous maintenance dose. In some embodiments, a treatment comprises administering intravenously an anti-Aβ protofibril antibody at 10 mg/kg, biweekly, e.g., for at least 18 months e.g., until a patient is amyloid-negative, before switching to a weekly, 360 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a weekly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a quarterly, 720 mg, subcutaneous maintenance dose.

In some embodiments, a patient will begin treatment comprising administering intravenously an anti-Aβ protofibril antibody at a dose of 10 mg/kg, then switch to a treatment comprising subcutaneously administering an anti-Aβ protofibril antibody, e.g., at a dose of 720 mg. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, e.g., for a total treatment period of at least 18 months or until a patient is amyloid-negative. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, before switching to a weekly, 360 mg, subcutaneous maintenance dose. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, before switching to a monthly, 720 mg, subcutaneous maintenance dose.

In some embodiments, the maintenance dose is administered as a subcutaneous injection of the anti-Aβ protofibril antibody (e.g., BAN2401). In some embodiments, the maintenance dose is administered as a weekly subcutaneous injection of the subcutaneous formulation of the anti-Aβ protofibril antibody. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation.

In some embodiments, the method of treatment comprises measuring the concentration of amyloid β 1-42 (Aβ42) and a concentration of amyloid $1-40 (Aβ40) in a first blood sample obtained from the subject to determine a first ratio of Aβ42 to Aβ40 (Aβ42/40 ratio). In some embodiments, the subject is then administered a therapeutically effective dose of an anti-amyloid β (A3) protofibril antibody. In some embodiments, a second blood sample is obtained after the first sample to determine a second Aβ42/40 ratio. In some embodiments, a second blood sample is obtained from a subject after treatment has stopped or been reduced. In some embodiments, a change in the Aβ42/40 ratio is used to determine a second therapeutically effective dose. In some embodiments, a subject having an elevated second ratio relative to the first ratio is administered a second therapeutically effective dose comprising the same or a lower amount of the anti-Aβ protofibril antibody than in the first dose to the subject. In some embodiments, a subject having a lower second ratio relative to the first ratio is administered a second therapeutically effective dose comprising a higher amount of the anti-Aβ protofibril antibody than in the first dose. In some embodiments, a subject having a lower second ratio relative to the first ratio is administered a different treatment for AD. A first therapeutically effective dose may be administered multiple times (e.g., biweekly or monthly for 6-18 months) before changing to a second therapeutically effective dose or dosing regimen after measuring a second Aβ42/40 ratio.

In some embodiments, a subject is administered a first dose of the anti-Aβ protofibril antibody without an initial titrating step up to the treatment dose (e.g., a subject starts treatment at 10 mg/kg with no titration). In some embodiments, a dose of BAN2401 may be used in treating AD without the need of a prior titrating step. In some embodiments, a subject is switched to a maintenance dose without an initial titrating step to the maintenance dose. Without being bound by theory, providing a therapeutic dose without a titration step may provide additional therapeutic benefits to the patient, e.g., a faster shift in plasma biomarkers toward amyloid negativity or facilitating identification sooner of patients that do not have a therapeutic change in plasma biomarkers in response to the anti-Aβ protofibril antibody (non-responders) and who would benefit from alternative treatment.

In some embodiments, the at least one anti-Aβ protofibril antibody is BAN2401, also known as lecanemab. The terms “BAN2401” and “lecanemab” are used interchangeably and refer to a humanized IgG1 monoclonal version of mAb158, which is a murine monoclonal antibody raised to target protofibrils and disclosed in WO 2007/108756 and Journal of Alzheimer's Disease 43: 575-588 (2015). BAN2401 comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3) and is described in WO 2007/108756 and in Journal of Alzheimer's Disease 43:575-588 (2015). BAN2401 comprises (i) a heavy chain variable region comprising the amino acid sequence of SEQ ID NO: 7 and (ii) a light chain variable region comprising the amino acid sequence of SEQ ID NO: 8. The full length sequences of heavy chain and light chain of BAN2401 are set forth in SEQ ID NOs: 9 and 10 and is described in WO 2007/108756 and in Journal of Alzheimer's Disease 43:575-588 (2015).

Other non-limiting examples of suitable antibodies for use as the at least one anti-Aβ protofibril antibody in the present disclosure include aducanumab, as well as those disclosed in WO 2002/003911, WO 2005/123775, WO 2007/108756, WO 2011/001366, WO 2011/104696, and WO 2016/005466.

In some embodiments, the anti-Aβ protofibril antibody is administered subcutaneously at a dose ranging from 300 mg to 800 mg, or from 400 to 1500 mg. In some embodiments, the anti-Aβ protofibril antibody is administered subcutaneously at a dose of 300 mg to 400 mg. In some embodiments, the anti-Aβ protofibril antibody is administered subcutaneously at a dose of 400 mg to 500 mg. In some embodiments, the anti-Aβ protofibril antibody is administered subcutaneously at a dose of 400 mg to 450 mg. In some embodiments, the anti-Aβ protofibril antibody is administered subcutaneously at a dose of 450 mg to 500 mg. In some embodiments, the anti-Aβ protofibril antibody is administered subcutaneously at a dose of 500 mg to 600 mg. In some embodiments, the anti-Aβ protofibril antibody is administered subcutaneously at a dose of 500 mg to 550 mg. In some embodiments, the anti-Aβ protofibril antibody is administered subcutaneously at a dose of 550 mg to 600 mg. In some embodiments, the anti-Aβ protofibril antibody is administered subcutaneously at a dose of 600 mg to 700 mg. In some embodiments, the anti-Aβ protofibril antibody is administered subcutaneously at a dose of 600 mg to 650 mg. In some embodiments, the anti-Aβ protofibril antibody is administered subcutaneously at a dose of 650 mg to 700 mg. In some embodiments, the anti-Aβ protofibril antibody is administered subcutaneously at a dose of 700 mg to 800 mg. In some embodiments, the anti-Aβ protofibril antibody is administered subcutaneously at a dose of 700 mg to 750 mg. In some embodiments, the anti-Aβ protofibril antibody is administered subcutaneously at a dose of 750 mg to 800 mg. In some embodiments, the anti-Aβ protofibril antibody is administered subcutaneously at a dose of 300 mg, 310 mg, 320 mg, 330 mg, 340 mg, 350 mg, 360 mg, 370 mg, 380 mg, or 390 mg. In some embodiments, the anti-Aβ protofibril antibody is administered subcutaneously at a dose of 400 mg, 410 mg, 420 mg, 430 mg, 440 mg, 450 mg, 460 mg, 470 mg, 480 mg, or 490 mg. In some embodiments, the anti-Aβ protofibril antibody is administered subcutaneously at a dose of 500 mg, 510 mg, 520 mg, 530 mg, 540 mg, 550 mg, 560 mg, 570 mg, 580 mg, or 590 mg. In some embodiments, the anti-Aβ protofibril antibody is administered subcutaneously at a dose of 600 mg, 610 mg, 620 mg, 630 mg, 640 mg, 650 mg, 660 mg, 670 mg, 680 mg, or 690 mg. In some embodiments, the anti-Aβ protofibril antibody is administered subcutaneously at a dose of 700 mg, 710 mg, 720 mg, 730 mg, 740 mg, 750 mg, 760 mg, 770 mg, 780 mg, or 790 mg. In some embodiments, the anti-Aβ protofibril antibody is administered subcutaneously at a dose of 440 mg. In some embodiments, the anti-Aβ protofibril antibody is administered subcutaneously at a dose of 580 mg. In some embodiments, the anti-Aβ protofibril antibody is administered subcutaneously at a dose of 720 mg.

In some embodiments, the anti-Aβ protofibril antibody is administered subcutaneously in a dose ranging from 800 mg to 1600 mg. In some embodiments, the anti-Aβ protofibril antibody is administered subcutaneously in a dose of 800 mg to 1000 mg. In some embodiments, the anti-Aβ protofibril antibody is administered subcutaneously at a dose of 800 mg to 900 mg. In some embodiments, the anti-Aβ protofibril antibody is administered subcutaneously at a dose of 900 mg to 1000 mg. In some embodiments, the anti-Aβ protofibril antibody is administered subcutaneously at a dose of 1000 mg to 1200 mg. In some embodiments, the anti-Aβ protofibril antibody is administered subcutaneously at a dose of 1000 mg to 1100 mg. In some embodiments, the anti-Aβ protofibril antibody is administered subcutaneously at a dose of 1100 mg to 1200 mg. In some embodiments, the anti-Aβ protofibril antibody is administered subcutaneously at a dose of 1200 mg to 1400 mg. In some embodiments, the anti-Aβ protofibril antibody is administered subcutaneously at a dose of 1200 mg to 1300 mg. In some embodiments, the anti-Aβ protofibril antibody is administered subcutaneously at a dose of 1300 mg to 1400 mg. In some embodiments, the anti-Aβ protofibril antibody is administered subcutaneously at a dose of 1400 mg to 1600 mg. In some embodiments, the anti-Aβ protofibril antibody is administered subcutaneously at a dose of 1400 mg to 1500 mg. In some embodiments, the anti-Aβ protofibril antibody is administered subcutaneously at a dose of 1500 mg to 1600 mg. In some embodiments, the anti-Aβ protofibril antibody is administered subcutaneously at a dose of 800 mg, 820 mg, 840 mg, 860 mg, 880 mg, 900 mg, 920 mg, 940 mg, 960 mg, or 960 mg. In some embodiments, the anti-Aβ protofibril antibody is administered subcutaneously at a dose of 1000 mg, 1020 mg, 1040 mg, 1060 mg, 1080 mg, 1100 mg, 1120 mg, 1140 mg, 1160 mg, or 1180 mg. In some embodiments, the anti-Aβ protofibril antibody is administered subcutaneously at a dose of 1200 mg, 1220 mg, 1240 mg, 1260 mg, 1280 mg, 1300 mg, 1320 mg, 1340 mg, 1360 mg, or 1380 mg. In some embodiments, the anti-Aβ protofibril antibody is administered subcutaneously at a dose of 1400 mg, 1400 mg, 1440 mg, 1460 mg, 1480 mg, 1500 mg, 1520 mg, 1540 mg, 1560 mg, or 1580 mg. In some embodiments, the anti-Aβ protofibril antibody is administered subcutaneously at a dose of 880 mg. In some embodiments, the anti-Aβ protofibril antibody is administered subcutaneously at a dose of 1160 mg. In some embodiments, the anti-Aβ protofibril antibody is administered subcutaneously at a dose of 1440 mg.

In some embodiments, the anti-Aβ protofibril antibody is in the form of a pharmaceutical composition. In some embodiments, the pharmaceutical composition comprising the anti-Aβ protofibril antibody is administered via one or more syringes and/or autoinjectors. In some embodiments, the pharmaceutical composition comprising the anti-Aβ protofibril antibody is administered into the abdomen.

In some embodiments, the anti-Aβ protofibril antibody is present in a pharmaceutical composition in a concentration of at least 80 mg/mL. In some embodiments, the anti-Aβ protofibril antibody is present in a pharmaceutical composition in a concentration of at least 100 mg/mL. In some embodiments, the anti-Aβ protofibril antibody is present in a pharmaceutical composition in a concentration of at least 200 mg/mL. In some embodiments, the anti-Aβ protofibril antibody is present in a pharmaceutical composition in a concentration of at least 250 mg/mL. In some embodiments, the antibody is present in a pharmaceutical composition in a concentration of 80 mg/mL to 300 mg/mL. In some embodiments, the anti-Aβ protofibril antibody is present in a pharmaceutical composition in a concentration of 85 mg/mL to 275 mg/mL. In some embodiments, the anti-Aβ protofibril antibody is present in a pharmaceutical composition in a concentration of 90 mg/mL to 250 mg/mL. In some embodiments, the anti-Aβ protofibril antibody is present in a pharmaceutical composition in a concentration of 95 mg/mL to 225 mg/mL. In some embodiments, the anti-Aβ protofibril antibody is present in a pharmaceutical composition in a concentration of 100 mg/mL to 200 mg/mL. In some embodiments, the anti-Aβ protofibril antibody is present in a pharmaceutical composition in a concentration of 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, or 300 mg/mL. In some embodiments, the anti-Aβ protofibril antibody is present in a pharmaceutical composition in a concentration of 100 mg/mL. In some embodiments, the anti-Aβ protofibril antibody is present in a pharmaceutical composition in a concentration of 200 mg/mL. In some embodiments, the anti-Aβ protofibril antibody is present in a pharmaceutical composition in a concentration of 250 mg/mL. In some embodiments, the anti-Aβ protofibril antibody is present in a pharmaceutical composition in a concentration of 300 mg/mL. In some embodiments, the anti-Aβ protofibril antibody is lecanemab.

In some embodiments, the pharmaceutical composition comprising an anti-Aβ protofibril antibody further comprises at least one additional component. In some embodiments, the at least one additional component in the pharmaceutical composition is chosen from pharmaceutically acceptable buffers. In some embodiments, the pharmaceutically acceptable buffer is a citrate buffer. In some embodiments, the pharmaceutically acceptable buffer is a histidine buffer. In some embodiments, the at least one additional component in the pharmaceutical composition is chosen from emulsifiers. In some embodiments, the at least one additional component in the pharmaceutical composition is chosen from citric acid (or citric acid monohydrate), sodium chloride, histidine (and/or histidine hydrochloride), arginine (and/or arginine hydrochloride), and polysorbate 80. In some embodiments, the at least one additional component in the pharmaceutical composition is chosen from citric acid (and/or citric acid monohydrate), arginine (and/or arginine hydrochloride), and polysorbate 80. In some embodiments, the at least one additional component in the pharmaceutical composition is chosen from histidine (and/or histidine hydrochloride), arginine (and/or arginine hydrochloride), and polysorbate 80.

In some embodiments, the pharmaceutical composition comprises arginine (and/or arginine hydrochloride). In some embodiments, the concentration of arginine (and/or arginine hydrochloride) in the pharmaceutical composition ranges from 100 mM to 400 mM. In some embodiments, the concentration of arginine (and/or arginine hydrochloride) in the pharmaceutical composition ranges from 110 mM to 380 mM, 120 mM to 360 mM, 125 mM to 350 mM, 140 mM to 340 mM, 160 mM to 325 mM, 175 mM to 300 mM, or 200 mM to 250 mM. In some embodiments, the concentration of arginine (and/or arginine hydrochloride) in the pharmaceutical composition ranges from 110 mM to 150 mM, 150 mM to 200 mM, 200 mM to 250 mM, 250 mM to 300 mM, 300 mM to 350 mM, or 350 mM to 380 mM. In some embodiments, the concentration of arginine (and/or arginine hydrochloride) is 125 mM. In some embodiments, the concentration of arginine (and/or arginine hydrochloride) is 200 mM. In some embodiments, the concentration of arginine (and/or arginine hydrochloride) is 350 mM.

In some embodiments, the pharmaceutical composition comprises histidine. In some embodiments, the concentration of histidine in the pharmaceutical composition ranges from 10 mM to 100 mM. In some embodiments, the concentration of histidine in the pharmaceutical composition ranges from 10 mM to 100 mM, 12 mM to 80 mM, 14 mM to 60 mM, 15 mM to 55 mM, 15 mM to 35 mM, or 15 mM to 25 mM. In some embodiments, the concentration of histidine is 25 mM. In some embodiments, the concentration of histidine is 50 mM.

In some embodiments, the pharmaceutical composition comprises polysorbate 80. In some embodiments, the concentration of polysorbate 80 in the pharmaceutical composition ranges from 0.01 to 0.1% w/v, 0.01 to 0.08% w/v, 0.02 to 0.08% w/v, 0.03 to 0.07% w/v, or 0.04 to 0.06% w/v. In some embodiments, the polysorbate 80 is present in the pharmaceutical composition in a concentration of 0.01% w/v, 0.02% w/v, 0.03% w/v, 0.04% w/v, 0.05% w/v, 0.06% w/v, 0.07% w/v, or 0.08% w/v. In some embodiments, the polysorbate 80 is present in the pharmaceutical composition in a concentration of 0.02% w/v. In some embodiments, the polysorbate 80 is present in the pharmaceutical composition in a concentration of 0.05% w/v.

In some embodiments, the pharmaceutical composition comprises citric acid monohydrate. In some embodiments, the concentration of citric acid monohydrate in the pharmaceutical composition ranges from 10 mM to 100 mM. In some embodiments, the concentration of citric acid monohydrate in the pharmaceutical composition ranges from 10 mM to 100 mM, 10 mM to 90 mM, 15 mM to 85 mM, 20 mM to 80 mM, 25 mM to 75 mM, 30 mM to 70 mM, 30 mM to 60 mM, or 30 mM to 50 mM. In some embodiments, the concentration of citric acid monohydrate in the pharmaceutical composition is 50 mM.

In some embodiments, the disclosure provides a pharmaceutical composition having a pH in the range of 4.5 to 5.5. In some embodiments, the pH in the pharmaceutical composition is in the range of 4.0 to 6.0, 4.2 to 5.8, 4.3 to 5.7, 4.4 to 5.6, or 4.5 to 5.5. In some embodiments, the pH is 4.5, 4.6, 4.7, 4.8, 4.9, 5.0, 5.1, 5.2, 5.3, 5.4 or 5.5. In some embodiments, the pH is 5.0.

In some embodiments, the pharmaceutical compositions disclosed herein may be in the form of a solution and/or any other suitable liquid formulation deemed appropriate by one of ordinary skill in the art. In some embodiments, the pharmaceutical composition is formulated as a sterile, non-pyrogenic liquid for subcutaneous administration. In some embodiments, the pharmaceutical composition is a saline solution.

In some embodiments, the pharmaceutical composition is a liquid dosage form comprising an anti-Aβ protofibril antibody that binds to Aβ protofibril, such as lecanemab, and further comprising, for instance, citric acid monohydrate, arginine, arginine hydrochloride, and polysorbate 80. In some embodiments, the pharmaceutical composition comprises 100 mg/mL of an anti-Aβ protofibril antibody that binds to Aβ protofibril, such as lecanemab, 50 mM citric acid monohydrate, 110 mM arginine, 240 mM arginine hydrochloride, and 0.05% (w/v) polysorbate 80, and has a pH of 5.0±0.4.

In some embodiments, the pharmaceutical composition is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 100 mg/mL or 200 mg/mL of an anti-Aβ protofibril antibody that binds to Aβ protofibril, such as lecanemab, 25 mM of histidine and histidine hydrochloride, 200 mM arginine hydrochloride, and 0.05% (w/v) polysorbate 80, and has a pH of 5.0±0.4. In some embodiments, the pharmaceutical composition comprises as a sterile aqueous solution 200 mg/mL lecanemab, 200 mM arginine, 25 mM histidine and histidine hydrochloride, 0.05% (w/v) Polysorbate 80.

In some embodiments, the pharmaceutical composition is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 200 mg/mL of an anti-Aβ protofibril antibody that binds to Aβ protofibril, such as lecanemab, 50 mM histidine and histidine hydrochloride, 125 mM arginine hydrochloride, and 0.02% (w/v) polysorbate 80, and has a pH of 5.0±0.4.

In some embodiments, the pharmaceutical composition is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 200 mg/mL of an anti-Aβ protofibril antibody that binds to Aβ protofibril, such as lecanemab, 50 mM citric acid (and/or citric acid monohydrate), 125 mM arginine (and/or arginine hydrochloride), and 0.02% (w/v) polysorbate 80, and has a pH of 5.0±0.4.

Lecanemab and methods comprising the use of lecanemab are disclosed in U.S. Provisional Application No. 62/749,614 and PCT International Application No. PCT/US2019/043067, both of which are incorporated herein by reference in their entireties.

Methods comprising the use of lecanemab in a subject having preclinical AD are disclosed in Clinical Trial Identifier: NCT04468659 (ClinicalTrials.gov), which are incorporated herein by reference in their entireties.

### Non-Limiting Embodiments of the Disclosure

Certain embodiments of the present disclosure relate to aqueous pharmaceutical formulations and methods of using such pharmaceutical formulations.

Some embodiments relate to a method comprising:

Embodiment 1: a method of treating Alzheimer's disease comprising subcutaneously administering to a subject in need thereof a suitable dose, such as 400 mg to 1500 mg or 400 mg to 800 mg. of an anti-Aβ protofibril antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

Embodiment 2: a method of delaying clinical decline comprising subcutaneously administering to in a subject in need thereof a suitable dose, such as 400 mg to 1500 mg or 400 mg to 800 mg, of an anti-Aβ protofibril antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

Embodiment 3: a method of reducing brain amyloid level comprising subcutaneously administering to a subject in need thereof a suitable dose, such as 400 mg to 1500 mg or 400 mg to 800 mg, of an antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

Embodiment 4: a method of converting an amyloid positive subject to amyloid negative comprising subcutaneously administering to the subject a suitable dose, such as 400 mg to 1500 mg or 400 mg to 800 mg, of an antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

Embodiment 5A: the method according to any one of embodiments 1 to 4, wherein the subject has been diagnosed as having early Alzheimer's disease. Embodiment 5B: the method according to any one of embodiments 1 to 4, wherein the subject has been diagnosed as having preclinical Alzheimer's disease.

Embodiment 6: the method according to any one of embodiments 1 to 4, wherein the subject has been diagnosed as having Alzheimer's disease.

Embodiment 7: the method according to any one of embodiments 1 to 4, wherein the subject is at risk of developing Alzheimer's disease.

Embodiment 8: the method according to any one of embodiments 1 to 7, wherein the anti-Aβ protofibril antibody is administered once weekly.

Embodiment 8b: the method according to any one of embodiments 1 to 8a, wherein the anti-Aβ protofibril antibody is administered as a single administration or as two administrations.

Embodiment 9: the method according to any one of embodiments 1 to 8, wherein the anti-Aβ protofibril antibody is administered at a dose of 300 mg to 400 mg, 400 mg to 500 mg, 500 mg to 600 mg, 600 mg to 700 mg, or 700 mg to 800 mg.

Embodiment 10a: the method according to any one of embodiments 1 to 9, wherein the anti-Aβ protofibril antibody is administered at a dose of 360 mg, 440 mg, 580 mg, or 720 mg.

Embodiment 10b: the method according to any one of embodiments 1 to 10a, wherein the anti-Aβ protofibril antibody is administered at a dose of 720 mg, 880 mg, 1160 mg, or 1440 mg.

Embodiment 11: the method according to any one of embodiments 1 to 10, wherein the anti-Aβ protofibril antibody comprising a heavy chain complementarity variable region comprising an amino acid sequence of SEQ ID NO: 1, and a light chain variable region comprising an amino acid sequence of SEQ ID NO: 2.

Embodiment 12: the method according to any one of embodiments 1 to 11, wherein the subject is ApoE4-positive.

Embodiment 13: the method according to any one of embodiments 1 to 12, wherein the anti-Aβ protofibril antibody is comprised in a pharmaceutical composition in the form of a syringe or autoinjector.

Embodiment 14: a method of treating Alzheimer's Disease comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 15: a method of treating preclinical Alzheimer's Disease comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 16A: a method of delaying clinical decline in a subject having Alzheimer's disease comprising subcutaneously administering to the subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 16B: a method of delaying clinical decline in a subject having early Alzheimer's disease comprising subcutaneously administering to the subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 17: a method of reducing brain amyloid level in a subject comprising subcutaneously administering to the subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 18: a method of converting a subject from amyloid positive to negative comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

Embodiment 19: a method of delaying the pathophysiological and clinical progression of Alzheimer's Disease comprising subcutaneously administering to a subject in need thereof an aqueous pharmaceutical composition comprising:
a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
b) 100 mM to 400 mM arginine and/or arginine hydrochloride;
c) 0.01% w/v to 0.1% w/v polysorbate 80; and
d) a pharmaceutically acceptable buffer;
wherein the pharmaceutical composition has a pH ranging from 4.5 to 5.5.

Embodiment 20: a method of preventing Alzheimer's Disease comprising subcutaneously administering to a subject in need thereof an aqueous pharmaceutical composition comprising:
a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
b) 100 mM to 400 mM arginine and/or arginine hydrochloride;
c) 0.01% w/v to 0.1% w/v polysorbate 80; and
d) a pharmaceutically acceptable buffer;
wherein the pharmaceutical composition has a pH ranging from 4.5 to 5.5.

Embodiment 21: the method of any one of embodiments 15 to 20, wherein the subject has intact cognition.

Embodiment 22: the method of any one of embodiments 15 to 21, wherein the subject has elevated amyloid.

Embodiment 23: the method of any one of embodiments 15 to 21, wherein the subject has intermediate amyloid.

Embodiment 24: the method of any one of embodiments 15 to 23, wherein the subject is subcutaneously administered one injection of the pharmaceutical composition weekly from week 0 though week 8, followed by two injections of the pharmaceutical composition weekly from week 10 through week 96, followed by two injections of the pharmaceutical composition.

Embodiment 25: the method of any one of embodiments 15 to 23, wherein the subject is subcutaneously administered the pharmaceutical composition comprising 440 mg, 580 mg, or 720 mg of the anti-Aβ protofibril antibody weekly from week 0 through week 216.

Embodiment 26: the method of any one of embodiments 15 to 23, wherein the subject is subcutaneously administered one injection of the pharmaceutical composition every two weeks from week 0 through week 4, followed by two injections of the pharmaceutical composition every two weeks from week 6 through week 212.

Embodiment 27: the method of any one of embodiments 15 to 23, wherein the subject is administered the pharmaceutical composition weekly for at least two years after administration of the first dose of the pharmaceutical composition to the subject.

Embodiment 28: the method of any one of embodiments 15 to 27, wherein the subject is administered the pharmaceutical composition for at least 4 years.

Embodiment 29a: the method of any one of embodiments 15 to 29, wherein the subject is administered maintenance doses of the pharmaceutical composition.

Embodiment 29b: The method of embodiment 29a, wherein the maintenance dose is administered once or multiple times.

Embodiment 29b: The method of any one of embodiments 29a-b, wherein the maintenance dose is administered at a dose frequency is selected to maintain a PET SUVr level achieved during treatment.

Embodiment 29d: The method of embodiment 29b, wherein the maintenance dose is administered at a dose frequency is selected to maintain a PET SUVr level at or below 1.17.

Embodiment 29e: The method of any one of embodiments 29b-d, wherein the maintenance dose is administered every three months or every 12 weeks.

Embodiment 29f: The method of any one of embodiment 29b-d, wherein the maintenance dose is administered every month or every 4 weeks.

Embodiment 29g: The method of embodiment 29b, wherein the maintenance dose is administered at a dose frequency selected to maintain a Aβ42/40 ratio achieved during treatment.

Embodiment 29h: The method of embodiment 29g, wherein the maintenance dose is administered at a dose frequency selected to maintain a Aβ42/40 ratio at or above 0.092.

Embodiment 29i: The method of any one of embodiment 29g-h, wherein the maintenance dose is administered every month or every 4 weeks.

Embodiment 29j: The method of any one of embodiment 29a-h, wherein the administration of a maintenance dose is stopped or decreased in frequency or the dose is lowered when a favorable biomarker is achieved.

Embodiment 29j: The method of any one of embodiment 29a-h, wherein the administration of a maintenance dose is increased in frequency or the dose is increased when a favorable biomarker becomes less favorable.

Embodiment 30: the method of any one of embodiments 15 to 30, wherein the subject is monitored for amyloid accumulation and development of neurofibrillary tangles based on a PET scan for tau, plasma and/or CSF biomarkers.

Embodiment 31: the method of any one of embodiments 15 to 23, wherein the subject is subcutaneously administered one injection of the pharmaceutical composition weekly from week 0 through week 8, followed by two injections of the pharmaceutical composition weekly from week 10 through week 96 weeks, followed by two injections of the pharmaceutical formulation every two weeks from week 98 through week 216.

Embodiment 32: the method of any one of embodiments 15 to 23, wherein the subject is subcutaneously administered two injections of the pharmaceutical composition from week 8 through week 94 and/or from week 98 through week 216.

Embodiment 33: the method of any one of embodiments 15 to 23, wherein the subject is subcutaneously administered the pharmaceutical composition comprising 440 mg, 580 mg, or 720 mg of the anti-Aβ protofibril antibody weekly from week 0 through week 96, followed by administration of said pharmaceutical composition every two weeks from week 98 through week 216.

Embodiment 34: the method of any one of embodiments 15 to 23, wherein the subject is subcutaneously administered one injection of the pharmaceutical composition every two weeks from week 0 through to week 8, followed by two injections of the pharmaceutical composition every two weeks from week 10 through to week 216.

Embodiment 35: the method of any one of embodiments 15 to 23, wherein the subject is subcutaneously administered the pharmaceutical composition comprising 440 mg, 580 mg, or 720 mg of the anti-Aβ protofibril antibody every two weeks from week 10 to through week 216.

Embodiment 36: the method of embodiment 35, wherein the subject is subcutaneously administered the pharmaceutical composition comprising 440 mg, 580 mg, or 720 mg of the anti-Aβ protofibril antibody every two weeks from week 10 to through week 212.

Embodiment 37: the method any one of embodiments 1 to 36, wherein the subject is 65 to 80 years old.

Embodiment 38: the method any one of embodiments 1 to 37, wherein the subject is 55 to 64 years old and has at least one risk factor chosen from:
(i) a first degree relative diagnosed with dementia onset before age 75;
(ii) at least one apolipoprotein E4 variant (APOE4) allele; and
(iii) elevated brain amyloid according to PET or cerebrospinal fluid (CSF) testing prior to said administration.

Embodiment 39: the method of any one of embodiments 1 to 38, wherein the subject has a Global Clinical Dementia Rating (CDR) score of 0 at prior to said administration.

Embodiment 40: the method of any one of embodiments 1 to 39, wherein the subject has a Mini-Mental State Examination (MMSE) score greater than or equal to 27, with educational adjustments, prior to said administration.

Embodiment 41: the method of any one of embodiments 1 to 40, wherein the subject has a Wechsler Memory Scale-Revised Logical Memory subscale II (WMS-R LM II) score prior to said administration of at least one standard deviation below age-adjusted mean in the WMS-IV LMII of less than or equal to 15 for a subject of age ranging from 50 to 64 years, of less than or equal to 12 for a subject of age ranging from 65 to 69 years, of less than or equal to 11 for a subject of age ranging from 70 to 74 years, of less than or equal to 9 for a subject of age ranging from 75 to 79 years, and of less than or equal to 7 for a subject of age ranging from 80 to 90 years.

Embodiment 42: the method any one of embodiments 24, 26, 31, 32, or 34, wherein the volume of the injection is 1.1 mL, 1.4 mL, or 1.8 mL.

Embodiment 43: The method of any one of the preceding embodiments, wherein administering to the subject a first therapeutically effective dose of an anti-Aβ protofibril antibody does not require a titration step.

Embodiment 44: The method of any one of the preceding embodiments, wherein risk or incidence of amyloid-related imaging abnormality edema/effusion (ARIA E) is reduced, e.g. compared with IV administration of the anti-Aβ protofibril antibody of which exposure and/or efficacy is or is expected to be equivalent.

### EXAMPLES

### Example 1: Preparation of Lecanemab Formulations

### Preparation of SC Formulations

The following materials were used in an exemplary SC formulations containing 200 mg/mL lecanemab, as shown in Table 1.

TABLE 1
Exemplary 200 mg/mL SC formulation comprising lecanemab.

| | Composition | | | | |
|---|---|---|---|---|---|
| Component | 1a | 1b | 2 | 3 | 4 |
| Lecanemab | 200 mg/mL | 200 mg/mL | 200 mg/mL | 200 mg/mL | 200 mg/mL |
| Histidine | Total 50 mM ¹ | Total 50 mM ¹ | Total 50 mM ¹ | — | Total 50 mM ¹ |
| Histidine HCl | | | | | |
| Citric Acid | — | — | — | 50 mM | — |
| Arginine HCl | 125 mM | 125 mM | 125 mM | 125 mM | 200 mM |
| Polysorbate 80 | 0.02%(w/v) | 0.02%(w/v) | 0.02%(w/v) | 0.02%(w/v) | 0.05%(w/v) |
| Methionine | — | 5 mM | — | — | — |
| Water for | QS | QS | QS | QS | QS |
| infection | | | | | |
| pH | 5.0 ± 0.4 | 5.0 ± 0.4 | 5.0 ± 0.4 | 5.0 ± 0.4 | 5.0 ± 0.4 |

¹ Total concentration as Histidine

Lecanemab at a target protein concentration of 200 mg/mL was prepared via tangential flow filtration (TFF) as summarized below. A separate TFF operation was performed to prepare lecanemab material in each formulation buffer, except for Compositions 1a and 1b. For two of the formulations, one TFF operation was performed, and the resulting concentrated material was split into two half-lots. A small quantity of sterile filtered material in each final formulation buffer was not filled at time zero, but was stored frozen at −20° C. to be filled into the appropriate container closures for syringe testing.

### Lecanemab Preparations

The process of protein concentration/diafiltration via TFF can be subdivided into 3 stages:
1. Concentration of the material to 100-150 mg/mL
2. Diafiltration (5×) against formulation buffer
3. Concentration to >200 mg/mL

The concentration/diafiltration step was performed using a Pall Centramate LV system installed with 0.02 m² of membrane area. The lecanemab material (pulled from GMP lot manufacture prior to polysorbate 80 (PS80) addition) was charged into the TFF system and a 10-15 fold concentration (stage 1) was performed. The material was then diafiltered against up to 5 diavolumes of the formulation buffer (stage 2), with pH and conductivity checks of the permeate being done to monitor diafiltration. After diafiltration, the material was further concentrated to the target protein concentration of 210 to 250 mg/mL (stage 3). The retentate was collected and samples were taken for protein concentration determination.

In preparing this formulation, the target protein concentration of 210 to 250 mg/mL was not reached due to high pressure in the TFF system. Therefore, the target protein concentration was achieved by using Millipore centrifugal filter units (30,000 MWCO). To perform this concentration step, filter units were equilibrated with the lecanemab formulation buffer, followed by centrifugation of the lecanemab material at 3600 RPM (˜3000×g) for 30 minutes intervals at 20° C., until the protein concentration in the retentate was expected to be greater than 200 mg/mL. The retentate was recovered from the filter units and pooled. After thorough mixing, the pooled retentate was sampled for protein concentration measurements.

After the protein was concentrated, a sample was taken from the pool and diluted 500-fold with the appropriate formulation buffer. The absorbance of the diluted sample at 280 nm and 320 nm was measured against the buffer blank. The final protein concentration adjustment was performed via dilution with the appropriate formulation buffer. Lastly, 10% PS80 solution was added to the lecanemab to achieve 0.02% PS80 in the final solution, and the protein solution was thoroughly mixed via end-over-end rotation.

Final lecanemab formulated material was filtered using 0.2 μm syringe filters, and subsequently filled into vials or pre-filled syringe (PFS). This step was performed aseptically in a biosafety cabinet. The resulting vials or PFS were placed in a freezer at −20° C. Vials were stored inverted, and PFS were stored horizontally in order to simulate worst case conditions.

### Preparation of IV Formulations

A lecanemab 10 mg/mL and two 100 mg/mL formulations for intravenous (IV) injection were manufactured by a conventional cGMP aseptic process for preparation of a sterile aqueous formulation. These IV injection were produced from the corresponding lecanemab drug substances formulation as follow without addition of any excipients and dilution.

The filtered lecanemab drug substance solution was aseptically filled into vials. The pooled drug substance underwent a bioburden reducing filtration step through a 0.2-pm filter. The final sterile filtration was performed through two 0.2-pm filters in series, and pre-and post-filtration filter integrity tests were conducted. The sterile bulk drug product was filled aseptically into vials. During the filling operation, filling accuracy was confirmed by measuring the vial fill weight. Filled vials were stoppered and then sealed with an aluminum overseal. After crimp capping, the product was stored at 5 3° C.

The composition of an IV formulation comprising 10 mg/mL lecanemab is shown in Table 2.

TABLE 2
10 mg/mL IV Formulation of Lecanemab.

| Component | |
|---|---|
| Lecanemab | 10 mg/mL |
| Sodium citrate/Citric acid buffer | 25 mM |
| Sodium Chloride | 125 mM |
| Polysorbate 80 | 0.02% (w/v) |
| Water for Injection | QS |
| pH | 5.7 |

The compositions of two IV formulations comprising 100 mg/mL lecanemab each are shown in Tables 3 (“IV Formulation A”) and 4 (“IV Formulation B”).

TABLE 3
100 mg/mL IV formulation comprising Lecanemab (IV Formulation A).

| Component | IV Formulation A |
|---|---|
| Lecanemab | 100 mg/mL |
| Citric acid buffer | 50 mM |
| Arginine | 110 mM |
| Arginine Hydrochloride | 240 mM |
| Polysorbate 80 | 0.05% (w/v) |
| Water for Injection | QS |
| pH | 5.0 |

TABLE 4
100 mg/mL IV Formulation comprising Lecanemab (IV Formulation B)

| component | IV Formulation B |
|---|---|
| Lecanemab | 100 mg/mL |
| Histidine | Total 25 mM |
| Histidine HCl | |
| Arginine HCl | 200 mM |
| Polysorbate 80 | 0.05%(w/v) |
| Water for infection | QS |
| pH | 5.0 ± 0.4 |

### Example 2: NHP Pharmacokinetic Study

Lecanemab was provided as a liquid formulation in 25 mM L-histidine, 200 mM L-arginine, 0.05% polysorbate 80, pH 5.0. The protein concentration was 204.3 mg/mL and was regarded as 200 mg/mL at calculation of dosing formulation.

For the dosing formulations, lecanemab was left at room temperature to thaw on the day of use. The dosing formulation for intravenous administration was prepared on the day of dosing under a UV cut-off fluorescent lamp. It was prepared in a clean bench using sterilized instruments as much as possible. The dosing formulation (10 mg/mL) was prepared by diluting lecanemab with water for injection. After preparation, the dosing formulation was transferred to a sterilized polypropylene (PP) container and covered with aluminum foil. Lecanemab was used for subcutaneous administration with no preparation.

Lecanemab was administered to 6 male cynomolgus monkeys (3 years of age and having a body weight of 2.4 to 3.4 kg) intravenously and subcutaneously at a dose of 10 mg/kg (3 animals/route). The study design is shown in Table 5.

TABLE 5
Study Details

| | | Dose | | | |
|---|---|---|---|---|---|
| | Test | levels | Dose volume | Conc. | Number of Animals |
| Route of administration | article | (mg/kg) | (mL/kg) | (mg/mL) | (Animal No.) |
| Intravenous | lecanemab | 10 | 1 | 10 | 3 males (10101 to |
| administration | | | | | 10103) |
| Subcutaneous | lecanemab | 10 | 0.05 | 200 | 3 males (10201 to |
| administration | | | | | 10203) |

Dose justification Intravenous and subcutaneous administrations at 10 mg/kg/day were selected to determine the pharmacokinetic (PK) parameters at a dose level used in a safety test and to compare the PK parameters after subcutaneous and intravenous administrations at the same dose level. For the subcutaneous administration: dosing was performed on the dorsal area of animals. A disposable syringe with a needle (1 mL, 27G, Terumo Corporation, Japan) was used for dosing. The test article was used as is after being returned to room temperature. The fur of the dorsal area was clipped with clippers before dosing. The dosing volume was 0.05 mL/kg (the dosing volume for each animal was calculated based on the body weight measured on the day of dosing).

For the intravenous administration: the dosing formulation was injected into the saphenous vein at a rate of 2 mL/min using a disposable syringe, an extension tube, and an indwelling needle (22G, Nipro Corporation, Japan). The dosing volume was 1 mL/kg (the dosing volume for each animal was calculated based on the body weight measured on the day of dosing).

### Pharmacokinetics

Single dose was selected to calculate PK parameters.

Blood samples (approximately 1 mL) were collected from the cephalic vein from all animals without anesthesia based on the following schedule:

For intravenous administration: Day 1 (the day of dosing; 5 times, predose, 5 minutes, 1, 2, and 8 hours after dosing), Day 2 (24 hours after dosing), Day 3 (48 hours after dosing), Day 5 (96 hours after dosing), Day 8 (168 hours after dosing), Day 15 (336 hours after dosing), Day 29 (4 weeks after dosing; 672 hours after dosing), Day 43 (6 weeks after dosing; 1008 hours after dosing), and Day 57 (8 weeks after dosing; 1344 hours after dosing).

For subcutaneous administration: Day 1 (the day of dosing; 4 times, predose, 2, 4, and 8 hours after dosing), Day 2 (24 hours after dosing), Day 3 (48 hours after dosing), Day 4 (72 hours after dosing), Day 5 (96 hours after dosing), Day 8 (168 hours after dosing), Day 15 (336 hours after dosing), Day 29 (4 weeks after dosing; 672 hours after dosing), Day 43 (6 weeks after dosing; 1008 hours after dosing), and Day 57 (8 weeks after dosing; 1344 hours after dosing).

Blood samples were transferred into blood-collecting vessels containing serum separator (Venoject II, Terumo Corporation) and were left to stand at room temperature for 30 to 60 minutes before centrifugation for serum collection. After centrifugation (approximately 1750×g for 10 minutes at approximately 4° C.), serum samples (0.1 mL or more×2 tubes) were separated into polypropylene (PP) tubes, cooled with dry ice, and stored at approximately −80° C. (actual range: −84.8 to −76.8° C.; acceptable range: −60° C. or below), and sent to a test site in a frozen condition packed with dry ice.

The concentrations of lecanemab in serum were determined by an ELISA method.

TABLE 6
Pharmacokinetic Parameters of Lecanemab.

| Pharmacokinetic | IV Administration | SC Administration |
|---|---|---|
| Parameters | 10 mg/kg | 10 mg/kg |
| tₘₐₓ (hour) | NA | 96.0 ± 63.5 |
| Cₘₐₓ (μg/mL) | NA | 94.8 ± 6.3 |
| t_{1/2} (hour) | 241.4 ± 49.5 | 270.9 ± 45.1 |
| AUC₍₀₋₁₎ (μg · h/mL) | 53,400 ± 9900 | 50,800 ± 4700 |
| AUC_{(0-24 h)} (μg · h/mL) | 5050 ± 1140 | 908 ± 279 |
| AUC_{(0-inf)} (μg · h/mL) | 55,100 ± 9100 | 52,900 ± 4100 |
| MRT_{(0-inf)} (hour) | 344 ± 50 | 439 ± 15 |
| CL (mL/h/kg) | 0.189 ± 0.029 | NA |
| Vₛₛ (mL/kg) | 65.1 ± 15.5 | NA |
| F (%) | NA | 95.9 |

Each value represents mean ± SD of 3 animals.
F was calculated by dividing mean AUC_{(0-inf)} after subcutaneous administration by that after intravenous administration.
AUC_{(0-inf)} = area under the concentration-time curve from zero time extrapolated to infinite time,
AUC₍₀₋ₜ₎ = area under the concentration-time curve from zero time to time of last quantifiable concentration,
AUC_{(0-24 h)} = area under the concentration-time curve from zero time to 24 hours,
CL = total clearance,
Cₘₐₓ = maximum observed concentration,
F = absolute bioavailability,
MRT_{(0-inf)} = mean residence time from zero time extrapolated to infinite time,
NA = not applicable,
t_{1/2} = terminal elimination phase half.

After a single intravenous administration of lecanemab, the serum concentration of lecanemab declined with mean t_{1/2} of 241.4 hours. Mean values for CL, Vₛₛ, AUC_{(0-inf)}, and MRT_{(0-inf)} were 0.189 mL/h/kg, 65.1 mL/kg, 55,100 μg·h/mL, and 344 hours, respectively.

After a single subcutaneous administration of lecanemab, the serum concentration of lecanemab peaked at 96.0 hours (48.0 to 168 hours), and mean t_{1/2} was 270.9 hours. Mean values for Cₘₐₓ, AUC_{(0-inf)}, and MRT_{(0-inf)} were 94.8 μg/mL, 52,900 μg·h/mL, and 439 hours, respectively. The F of lecanemab was 95.9%.

### Anti-Drug Antibody (ADA) Analysis

Blood collection: Blood samples (approximately 1 mL) were collected from the cephalic vein from all animals without anesthesia on Day 1 (the day of dosing; predose), Day 29 (4 weeks after dosing; 672 hours after dosing), and Day 57 (8 weeks after dosing; 1344 hours after dosing).

Method of serum sample preparation: Blood samples were transferred into blood-collecting vessels containing serum separator (Venoject II, Terumo Corporation) and were left to stand at room temperature for 30 to 60 minutes before centrifugation for serum collection. After centrifugation (approximately 1750×g for 10 minutes at approximately 4° C.), serum samples (0.1 mL or more x 2 tubes) were separated into PP tubes, cooled with dry ice and stored at approximately −80° C. (actual range: −84.8 to −76.8° C.; acceptable range: −60° C. or below), and sent to the test site (Analytical Research Center, Shimura Laboratory, LSI Medience Corporation) in a frozen condition packed with dry ice.

ADA analysis: Anti-lecanemab antibody in serum were determined by a bridging electrochemiluminescent immunoassay (ECL) method in the test site.

In the screening assay of anti-lecanemab antibody, 1 analytical sample before dosing, 4 analytical samples on Day 29, and 4 analytical samples on Day 57 were judged to be potentially positive.

The potentially anti-lecanemab antibody positive 9 samples were subjected to confirmation assay. In the confirmation assay of anti-lecanemab antibody, 1 analytical sample on Day 29 and 4 analytical samples on Day 57 were judged to be positive. Therefore, titration assay was conducted for ADA analytical samples defined as positive.

In the titration assay of anti-lecanemab antibody, the antibody titer was 1 to 256.

### Other Observations

There were no lecanemab-related changes in clinical signs, body weight, and food consumption in any animals.

### Conclusion

PK profiles of lecanemab after single intravenous and subcutaneous administrations at a dose of 10 mg/kg were investigated in male cynomolgus monkeys (n=3/group).

After a single intravenous administration of lecanemab, PK profile of lecanemab in serum was characterized as low CL (mean value, 0.189 mL/h/kg) and low Vₛₛ (mean value, 65.1 mL/kg), and mean t_{1/2} was 241.4 hours. After a single subcutaneous administration, the serum concentration of lecanemab peaked at 96.0 hours, and mean t_{1/2} was 270.9 hours. Mean t_{1/2} values were comparable between intravenous and subcutaneous administrations. The F after subcutaneous administration was 95.9%. As for the ADA analysis, the anti-lecanemab antibody was detected at 1 analytical sample on Day 29 after subcutaneous administration and 4 analytical samples on Day 57 after intravenous and subcutaneous administrations (2 samples/route).

### Example 3: Toxicology Study

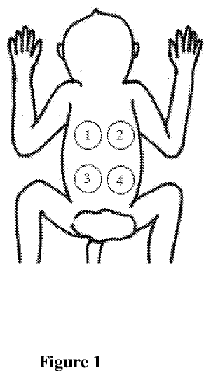

In order to assess local irritation effects, lecanemab was administered subcutaneously once a day for 4 weeks (28 days) to male and female cynomolgus monkeys (4 animals/group/sex) at a dose of 10 mg/kg (concentration: 200 mg/mL as lecanemab). Lecanemab was injected to 4 different dorsal areas every day for 4 weeks; i.e., site No. 1→2→3→1→2→3→4 for 4 weeks (FIG. 1); which allowed an assessment of acute local effect as well as its reversibility. A control group (4 animals/group/sex) received an equivalent volume (0.05 mL/kg) of control article (placebo [25 mM L-histidine; 200 mM L-arginine; 0.05% polysorbate 80]). All animals were necropsied 3 days after the final administration in Week 4.

Assessment of toxicity was based on mortality, clinical signs, including observation of the injection sites, body weights, food consumption, hematology, blood chemistry, toxicokinetics (TK), anti-drug antibody (ADA) analysis, macroscopic examination, and microscopic examination of the injection sites, axillary lymph node, inguinal lymph node, and spleen.

There were no deaths or test article-related changes in any of the assessment.

In TK, the mean Cₘₐₓ and AUC_{(0-24 h)} values were increased by repeated dosing with no apparent sex differences.

TABLE 7
Toxicokinetic Summary

| | | Male | | | |
|---|---|---|---|---|---|
| Dose | | Cₘₐₓ | tₘₐₓ | AUC_{(0-24 h)} | AUC_{(0-72 h)} |
| (mg/kg) | Day | (ug/mL) | (hour) | (μg · h/mL) | (μg · h/mL) |
| 10 | 1 | 52.4 ± 11.6 | 24.0 ± 0.0 | 657 ± 145 | NA |
| | 28 | 1470 ± 110 | 1.50 ± 1.73 | 32,000 ± 1300 | 92,900 ± |
| | | | | | 3500 |
| | | Female | | | |
| Dose | | Cₘₐₓ | tₘₐₓ | AUC_{(0-24 h)} | AUC_{(0-72 h)} |
| (mg/kg) | Day | (ug/mL) | (hour) | (μg · h/mL) | (μg · h/mL) |
| 10 | 1 | 54.6 ± 15.1 | 20.0 ± 8.0 | 797 ± 366 | NA |
| | 28 | 1610 ± 170 | 5.00 ± 3.83 | 32,600 ± 4000 | 90,100 ± |
| | | | | | 9300 |

Data represent the mean ± SD of 4 animals.
NA: not applicable.

In ADA analysis, all the applicable ADA analytical samples were judged to be negative.

The results indicated that a daily, subcutaneous administration of 10 mg/kg/day of lecanemab (200 mg/ml formulation) was well tolerated at over 28 days with no local irritation.

### Example 4: Subcutaneous Treatment Study Protocol

This study is a single-center, randomized, open-label, parallel-group study that was conducted in healthy subjects. This study evaluated the absolute bioavailability of lecanemab following a single fixed dose administered subcutaneously compared with a single intravenous dose. A total of 59 healthy subjects between 18 and 65 years of age were enrolled to support completion of at least 24 subjects for each treatment arm. Five Japanese subjects were included in the subcutaneous treatment arm only.

### Study Phases

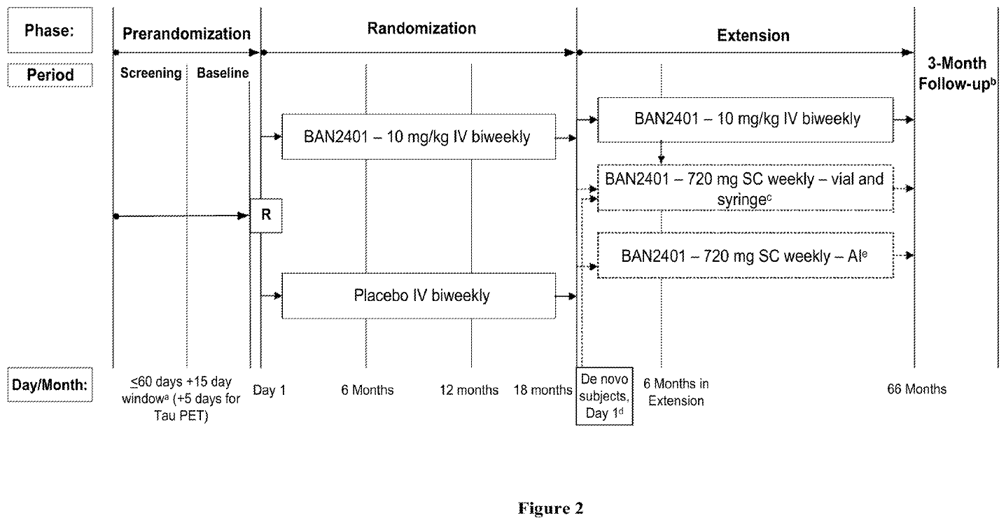

As shown in FIG. 2, the study consisted of 2 phases: a Prerandomization Phase and a Randomization Phase.

The Prerandomization Phase lasted up to 21 days and consisted of the Screening Period and the Baseline Period, during which each subject's study eligibility will be determined and baseline assessments will be conducted. The Screening Period lasted for 20 days and the Baseline Period will last 1 day (Day −1).

The Randomization Phase consisted of a Treatment Period and a Follow-up Period. Study treatment took place on Day 1 after subject study eligibility were confirmed and baseline assessments were conducted. Subjects were randomized in a 1:1 ratio to 1 of 2 treatment groups (A or B).

Test drug: Lecanemab drug product was supplied as a sterile aqueous solution comprising 200 mg/mL lecanemab with 200 mM arginine/25 mM histidine/0.05% Polysorbate 80, in glass vials containing 2 mL solution. Lecanemab was administered on a mg/kg basis for intravenous infusion, while a fixed dose of 700 mg will be used for subcutaneous administration.

Treatment A: 10 mg/kg IV lecanemab infusion over approximately 1 hour. Lecanemab was administered in normal saline over approximately 1 hour via intravenous infusion using an infusion system containing a terminal 0.2-μM in-line filter. Serum concentrations of lecanemab was measured at predetermined time points. A final Follow-Up visit took place on the last day of PK sample collection on Day 50.

Treatment B: Fixed 700 mg SC lecanemab subcutaneously administered in the abdomen (2 injections of 1.75 mL containing 350 mg each (i.e., concentration of 200 mg/mL)). Subcutaneous doses were administered via syringe; 2 subcutaneous injections were administered as one injection in each lower abdominal quadrant to achieve the full subcutaneous dose.

### Pharmacokinetic Assessments

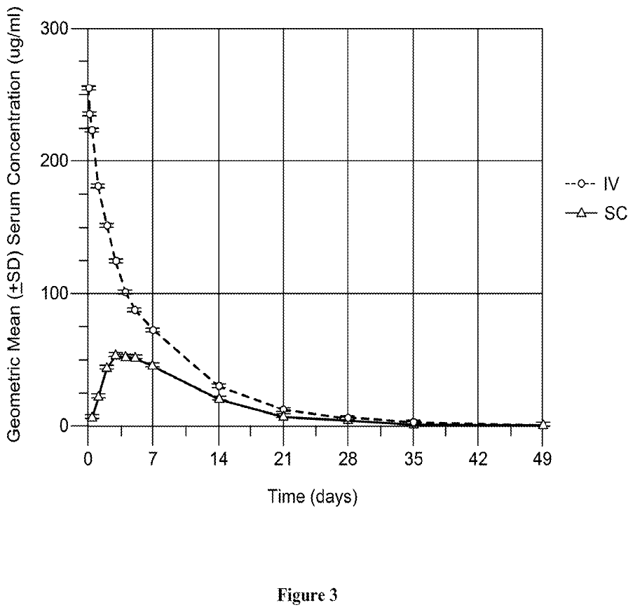

Serum samples for determination for lecanemab was collected after either intravenous or subcutaneous dosing on Day 1 at predose and postdose at 1 (IV: end of intravenous infusion and SC: 1 hour postdose), 2, 4, 8 hours, and on Day 2 (24 h postdose), Day 3 (48 hours), Day 4 (72 hours), Day 5 (96 hours), Day 6 (120 hours), Day 8 (168 hours), Day 15 (336 hours), Day 22 (504 hours), Day 29 (672 hours), Day 36 (840 hours), Day 50 (1176 hours), and any early termination (ET) visit for all subjects. See FIG. 3. All PK sampling timepoints are based on the start of IV infusion/SC injection.

TABLE 8
Pharmacokinetic Parameters

| | | | Bioavailability |
|---|---|---|---|
| Parameters | IV (n = 30) | SC (n = 29) | (%)^{a} |
| Mean Dose (mg) | 778 | 700 | |
| Dose Normalized | 0.340 (19.2) | 0.0854 (36.5) | |
| Cₘₐₓ (ug/mL/mg) | | | |
| Dose Normalized | 46.6 (28.4) | 24.3 (39.3) | 51.4 |
| AUC₍₀₋ₜ₎ | | | |
| (h* ug/mL/mg) | | | |
| Dose Normalized | 47.1 (29.2) | 23.8 (37.4)^{b} | 49.7 |
| AUC_{(0-inf)} | | | (90% CI: |
| (h*ug/mL/mg) | | | 43.54-56.83) |
| t_{1/2} (h) | 160 (29.2) | 150 (25.3)^{b} | |
| tₘₐₓ (h) | 2.00 | 72.20 | |
| | (1.00-8.00) | (47.50-168.00) | |

PK parameters presented as Geometric Mean (CV %) except for tmax as median (min-max)
IV: Intravenous;
SC: Subcutaneous;
F: Bioavailability;
^{a}based on analysis of variance (ANOVA);
^{b}n = 27

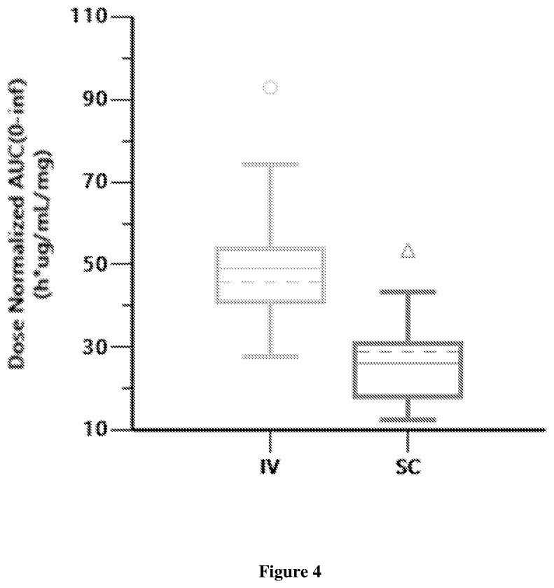

The absolute bioavailability following an SC dose was demonstrated to be approximately 50%. See FIGS. 3 and 4. Based on the bioavailability of ˜50%, a 700 mg weekly SC dose is predicted to in equivalent exposure to 10 mg/kg Q2W.

For the autoinjector (AI) device development, 3 injection volumes were preselected to accelerate technical development of the AI device (1.1, 1.4 or 1.8 mL). The 1.8 mL (360 mg) fill volume delivers 720 mg dose which is ˜3% higher than the projected dose. Taking into account the slight adjustment for the AI device, 720 mg QW is proposed as the SC dose regimen for future SC development.

No noticeable difference was observed between Japanese and Non-Japanese subjects.

### Safety Assessments

Safety assessments consisted of monitoring and recording all AEs; laboratory evaluation for hematology, blood chemistry, and urine values; periodic measurement of vital signs and electrocardiograms (ECGs); and the performance of physical examinations. Any adverse events (AEs) of injection site reactions were actively solicited and graded by the Common Toxicity Criteria (CTC). The clinical features of injection site reaction (pain, tenderness, erythema/redness, induration/swelling) were graded according to Table 9.

TABLE 9
Pain Grading for Injections

| Local | | | | Potentially |
|---|---|---|---|---|
| Reaction to | | | | Life |
| Injectable | Mild | Moderate | Severe | threatening |
| Product | (Grade 1) | (Grade 2) | (Grade 3) | (Grade 4) |
| Pain | Does not | Repeated use | Any use of | ER visit or |
| | interfere | of non- | narcotic | hospitalization |
| | with | narcotic pain | pain reliver | |
| | activity | reliever >24 | or prevents | |
| | | hours or | daily | |
| | | interferes | activity | |
| | | with activity | | |
| Tenderness | Mild | Discomfort | Significant | ER visit or |
| | discomfort | with | discomfort | hospitalization |
| | to touch | movement | at rest | |
| Erythema/ | 2.5-5 cm | 5.1-10 cm | >10 cm | Necrosis or |
| Redness^{a} | | | | exfoliative |
| | | | | dermatitis |
| Induration/ | 2.5-5 cm | 5.1-10 cm | >10 cm or | Necrosis |
| Swelling^{b} | and does not | or interferes | prevents | |
| | interfere | with activity | daily | |
| | with activity | | activity | |

ER = emergency room
^{a}In addition to grading measured local reaction at the greatest single diameter, the measurement should be recorded as continuous variable.
^{b}Induration/Swelling should be evaluated and graded using the functional scale as well as the actual measurement.

The injection site reaction at the injection site of each dose were graded as per Table 9 at each subsequent visit until resolution. No new or unexpected safety signals were detected with the SC formulation.

### Immunogenicity Assessment

Anti-drug (lecanemab) antibody (ADA) assessments in serum was conducted predose on Day 1, Day 15, Day 29, Day 50, and at any ET visit. If a subject was confirmed ADA positive with titer then samples were collected up to 6 months (every 3 months) until the ADA titers returned to baseline.

### Bioanalytical Methods

Serum concentrations of lecanemab were measured by the validated immunoprecipitation-liquid chromatography-tandem mass spectrometry (IP/LC-MS/MS) method using anti-human immunoglobulin G (IgG) antibody to precipitate lecanemab from a serum sample. Precipitated lecanemab was isolated and underwent proteolytic enzyme digestion to yield smaller peptides. The amount of peptide with a sequence unique to lecanemab was measured by liquid chromatography-tandem mass spectrometry (LC MS/MS) to provide a quantification of lecanemab.

ADA and neutralizing antibodies (NAb) were measured using validated ECL methods.

### Study Endpoints

The primary endpoints included the following PK parameters derived by noncompartmental analysis using the serum concentration-time data of lecanemab.

TABLE 10
PK parameters primary endpoints

| | |
|---|---|
| AUC₍₀₋ₜ₎ | Area under the concentration-time curve from time |
| | zero to time of last quantifiable concentration |
| AUC_{(0-72 h)} | Area under the concentration-time curve from time |
| | zero to 72 hours post end of IV infusion or SC |
| | administration |
| AUC_{(0-inf)} | Area under the concentration-time curve from time |
| | zero to time extrapolated to infinity |
| F | Absolute bioavailability of SC formulation |
| Cₘₐₓ | Maximum observed drug concentration |
| tₘₐₓ | Time to reach maximum (peak) concentration |
| | following drug administration |
| t_{1/2} | Terminal elimination half-life (if data permit) |

The primary PK parameters to evaluate bioavailability were AUC_{(0-inf)} and F=absolute bioavailability=[AUC_{(0-inf)} SC×Dose (IV)]/[AUC_{(0-inf)} IV×Dose (SC)]. IV dose was based on total dose (mg) infused.

Safety endpoints included the incidence of AEs, laboratory parameters, vital signs, ECG parameters, and serum ADA concentration.

### Safety Analyses

Evaluations of safety will be performed on the Safety Analysis Set. Safety data that will be evaluated include adverse events (including treatment emergent adverse events [TEAEs]), clinical laboratory results, vital signs, and ECGs and summarized by treatment group. Local injection site reactions will be analyzed as events of interest.

The number (percentage) of subjects with positive and negative ADA and ADA titer categories (e.g.: >0, 5, 25, 125), and NAb by visit will be summarized by treatment group. In addition, the correlation between ADA titer and PK profile will be evaluated (at the minimum) using descriptive statistics and summary plots if data permit.

TABLE 11
Schedule of Procedures/Assessments in Study Lecanemab

| | Prerandomization | Randomization | | | | | | | | | | | | | |
|---|---|---|---|---|---|---|---|---|---|---|---|---|---|---|---|
| | Period | | | | | | | | | | | | | | |
| | Screening | Baseline | Treatment | Follow-up | | | | | | | | | | | |
| | Day (s) | | | | | | | | | | | | | | |
| | −21 to −2 | −1^{a} | 1 | 2 | 3 | 4 | 5 | 6 | 8 | 15 | 22 | 29 | 36 | 50 | ET^{b} |
| Assessments | | | | | | | | | | | | | | | |
| Informed consent^{c} | X | | | | | | | | | | | | | | |
| Inclusion/exclusion | X | X | | | | | | | | | | | | | |
| criteria | | | | | | | | | | | | | | | |
| Demography | X | | | | | | | | | | | | | | |
| Medical history | X | | | | | | | | | | | | | | |
| Complete physical | X | | | | | | | | | | | | | | |
| exam | | | | | | | | | | | | | | | |
| Routine physical | | X | | | | | | | | | | | | | |
| exam | | | | | | | | | | | | | | | |
| Vital signs, height, | X | X | X | | X | X | X | X | X | X | X | X | X | X | X |
| weight^{d, e} | | | | | | | | | | | | | | | |
| Single 12-lead | X | X | X | | X | | | X | | | | | | X | X |
| ECG^{d, f} | | | | | | | | | | | | | | | |
| Viral Screen | X | | | | | | | | | | | | | | |
| (HAV-IgM, | | | | | | | | | | | | | | | |
| HBsAg, HCVAb) | | | | | | | | | | | | | | | |
| Urine drug, breath | X | X | | | | | | | | | | | | | |
| alcohol and | | | | | | | | | | | | | | | |
| tobacco screen^{g} | | | | | | | | | | | | | | | |
| Serum β-hCG | X | | | | | | | | | | | | | | |
| (females only) | | | | | | | | | | | | | | | |
| Urine pregnancy | | X | | | | | | | | | | | | X | |
| test (females only) | | | | | | | | | | | | | | | |
| Clinical labs | X | X | | | | | | | | | X | | | | |
| (hematology, | | | | | | | | | | | | | | | |
| clinical chemistry, | | | | | | | | | | | | | | | |
| urinalysis)^{d, h} | | | | | | | | | | | | | | | |
| Blood for serum | | X | X | | | | | | | X | | X | | X | X |
| anti-drug antibodyⁱ | | | | | | | | | | | | | | | |
| PK blood | | | X | X | X | X | X | X | X | X | X | X | X | X | X |
| sampling for | | | | | | | | | | | | | | | |
| lecanemabⁱ | | | | | | | | | | | | | | | |
| Randomization | | | X | | | | | | | | | | | | |
| lecanemab | | | X | | | | | | | | | | | | |
| administration | | | | | | | | | | | | | | | |
| Admission to | | X | | | | | | | | | | | | | |
| clinic (Inpatient) | | | | | | | | | | | | | | | |
| Discharge from | | | | X | | | | | | | | | | | |
| clinic (Outpatient) | | | | | | | | | | | | | | | |
| Outpatient visits | | | | | X | X | X | X | X | X | X | X | X | X | |
| Discharge from | | | | | | | | | | | | | | | X |
| study | | | | | | | | | | | | | | | |
| Adverse Events | < - - - > | | | | | | | | | | | | | | |
| Concomitant | < - - - > | | | | | | | | | | | | | | |
| Medications | | | | | | | | | | | | | | | |

β-hCG = human chorionic gonadotropin,
ET = Early Termination,
FU = follow up,
HBsAg = hepatitis B surface antigen,
HCVAb = hepatitis C virus antibody,
PK = pharmacokinetics,
HAV-IgM= anti-hepatitis A virus IgM
^{a}Subjects will be admitted to clinic on Day −1 until the morning of Day 2.
^{b}Procedures only to be conducted in the event of early termination from the study.
^{c}Informed consent must be obtained before any other study procedures or assessments.
^{d}At time points when vital signs, ECGs, blood sampling, or meals coincide, these procedures will be performed in the following order: ECGs, vital signs, blood sampling, then meals.
^{e}Vital signs (blood pressure, heart rate, body temperature, respiratory rate) will be recorded at Screening, Day −1, and at the Follow-up/ET Visit(s). In addition, vital signs will be obtained predose and 4 hours postdose on Day 1, relative to dosing. Subjects will need to rest in a supine position for 10 minutes before and 5 minutes after vital signs are taken. Height and weight will be recorded at Screening, and weight will be recorded at Baseline and at the FU/ET Visits.
^{f}Single 12-lead ECGs will be taken at Screening, Baseline, and ET Visit (if applicable). In addition, single 12-lead ECGs will be obtained predose and 4 hours postdose on Day 1 and on each follow up visit, except Day 8. Subjects will need to rest in a supine position for 10 minutes before initiation and 5 minutes after completion of an ECG recording.
^{g}Urine test for drugs of abuse, alcohol breathalyzer test, and urine cotinine test must be negative at Screening and at Baseline. Random drug, nicotine, and alcohol testing may be done at any time during the study, per the discretion of the investigator or sponsor.
^{h}Subjects will fast for at least 4 hours before blood is drawn for clinical laboratory assessments.
ⁱBlood samples for determination of anti-drug (lecanemab) antibodies will be taken predose on Day 1 and at Day 15, Day 29, Day 50, and at Early Termination visit (if applicable).
^{j}Blood samples for determination of serum lecanemab will be collected on Day 1 at predose and postdose (end of IV infusion) at 1, 2, 4, 8 hours, and on Day 2 (24 h postdose), Day 3, Day 4, Day 5, Day 6, Day 8 (168 h), Day 15, Day 22, Day 29, Day 36, Day 50, and at any Early Termination visit
^{k}Lecanemab will be administered on Day 1 either as intravenous or subcutaneous based on randomization scheme.

### Bioequivalence Simulations

Simulations were conducted using a population modeling approach to support bioequivalence.

In a first simulation, an IV dose (10 mg/kg single dose infused over 1 hour) and an SC dose (a 550 mg fixed dose administered weekly) were compared in parallel.

TABLE 12
Predicted Exposures for AUC &Cₐᵥ at Steady State

| | Cav | Cmax | Cmin |
|---|---|---|---|
| Dose | (ug/mL) | (ug/mL) | (ug/mL) |
| 10 mg/kg IV Q2W | 115 | 286 | 57 |
| (Amount = 700 mg) | | | |
| 550 mg SC QW | 114 | 123 | 100 |
| Ratio SC/IV | 0.99 | 0.430 | 1.75 |

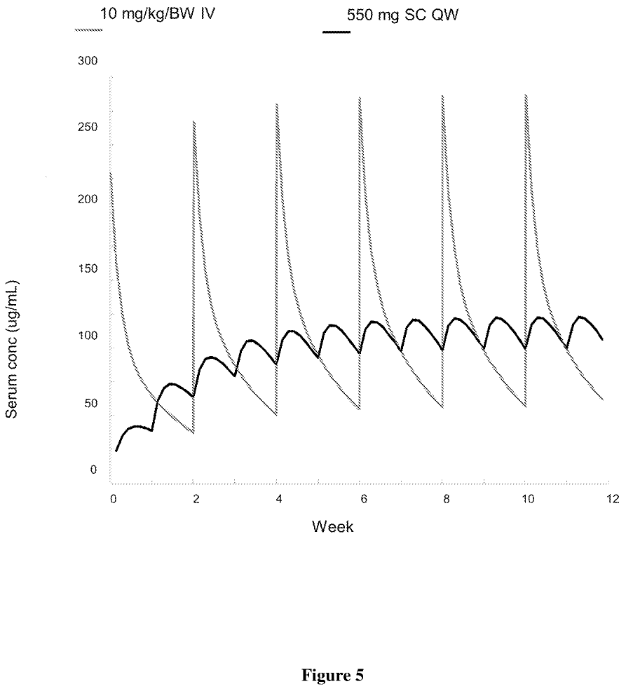

Comparable efficacy was predicted for the SC treatment and the IV treatment. See Table 12 and FIG. 5.

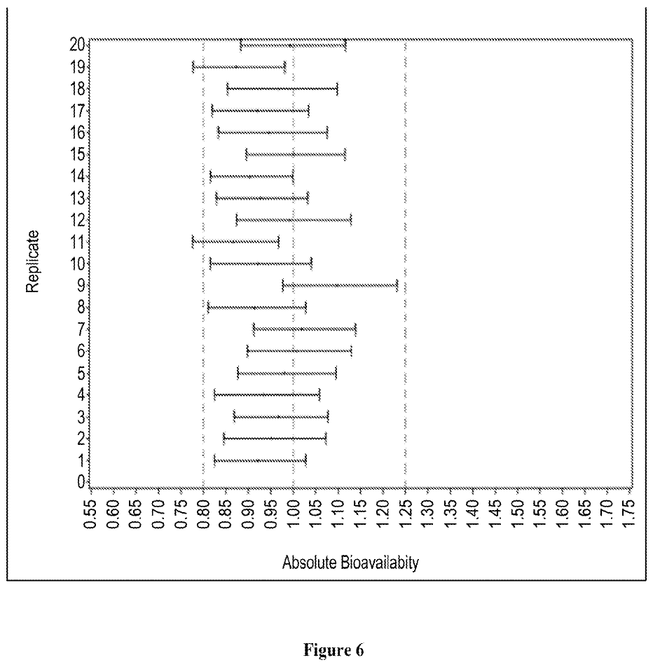

The predicted ratio of geometric mean and associated 90% CI fall within 80%-125%. See FIG. 6. Bioequivalence was established following a single dose to establish a dose of 720 mg.

In a second simulation, an IV dose (10 mg/kg single dose infused over 1 hour) and an SC dose (two 720 mg fixed doses administered one week apart) were compared. Body weight and gender were resampled and 60 subjects were analyzed in 20 replicates. See Table 13.

TABLE 13
Comparable Exposures for AUC &Cₐᵥ at Steady State

| | AUC | Cav | Cmax | Cmin |
|---|---|---|---|---|
| Dose | (ug*h/mL) | (ug/mL) | (ug/mL) | (ug/mL) |
| 10 mg/kg IV Q2W | 39400 | 117 | 283 | 60.6 |
| (Amount = 700 mg) | | | | |
| 720 mg SC QW | 40300 | 120 | 130 | 103 |
| (2 × 1.8 mL) | | | | |
| Ratio SC/IV | 1.02 | 1.02 | 0.460 | 1.70 |

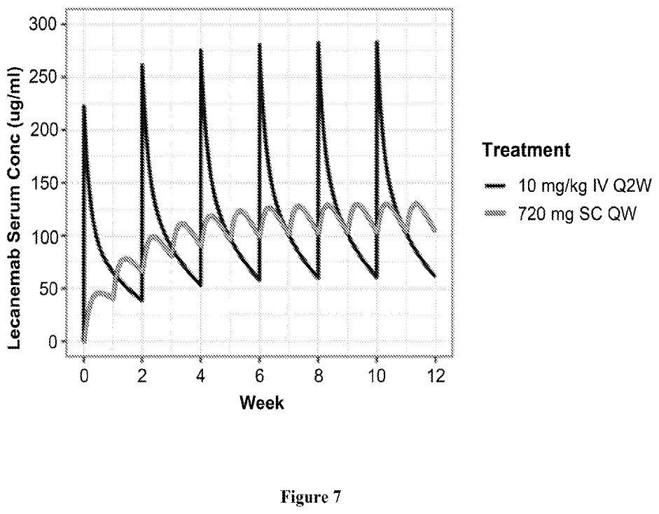

Comparable AUC for the SC treatment and the AUC for the IV treatment was achieved in approximately 4 weeks. See Table 14 and FIG. 7.

TABLE 14
AUC ratio over 6 weeks

| Weeks | AUC ratio SC/IV |
|---|---|
| 0-2 | 0.684 |
| 2-4 | 0.928 |
| 4-6 | 0.991 |

Simulations support that bioequivalence was achieved between IV (single dose) and SC (2 doses administered a week apart). The AUC for the SC dose adjusted to 2×720 mg dose resulted in bioequivalence to the IV dose (CI 0.88-1.17).

### Exposure Modeling

Pharmacokinetics(PK)/pharmacodynamics (PD) simulations were conducted to evaluate the effect of differences in lecanemab exposures that are anticipated at low/high body weight extremes on lecanemab efficacy and safety. PK simulations were performed using the PK model for subjects with EAD to explore the impact of body weight on the AUCss of lecanemab when administered as a fixed subcutaneous dose and body-weight based intravenous dose.

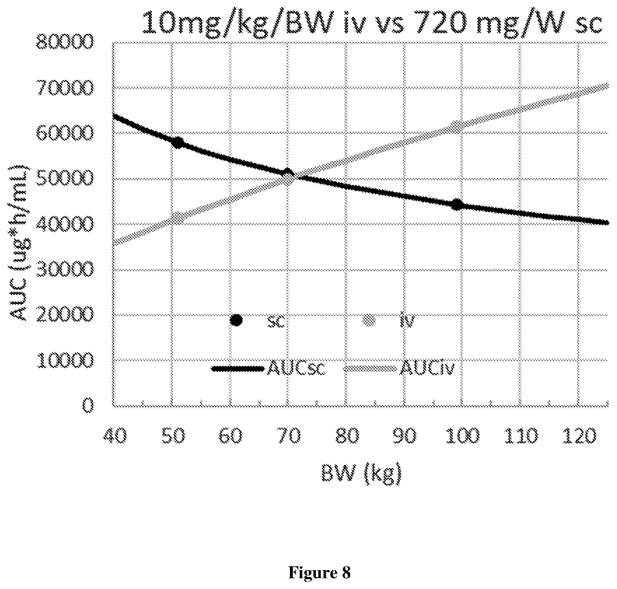

As shown in FIG. 8, lecanemab exposure shows a relative increase with increase in body weight following intravenous dose administration; in contrast, lecanemab exposure shows a relative decrease with increase in body weight for the fixed subcutaneous dose.

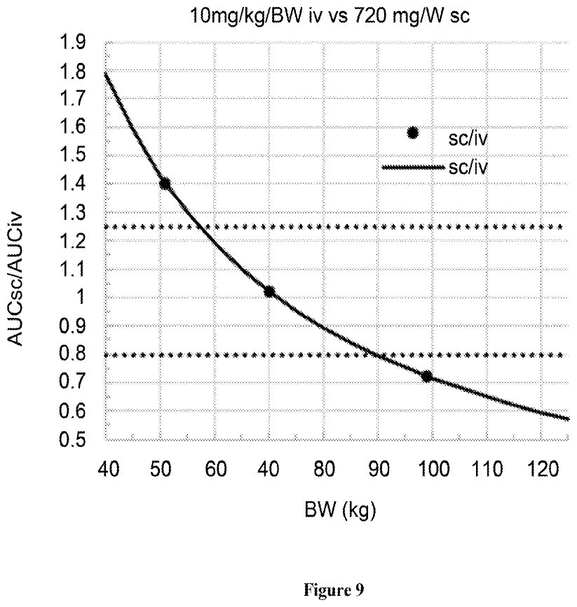

However, as shown in FIG. 9, for a wide range of body weights (approximately 58-90 kg,) lecanemab exposure is equivalent (CI within 80-125%) for intravenous and subcutaneous administration. The AUCₛₛ ratio is higher than 1.25 for subjects with low body weight such as 51 kg (5th percentile of PK analysis set) and is slightly lower than 0.8 for subjects with high body weight such as 99 kg (95th percentile of PK analysis set). See Table 14.

TABLE 14
AUC ratio for different body weight

| Weight | AUC ratio SC/IV |
|---|---|
| 51 kg | >1.25 |
| | (AUC for SC~40% higher) |
| 57 kg-90 kg | 0.8-1.25 |
| 99 kg | <0.80 |
| | (AUC for SC~28% lower) |

### Safety and Efficacy Modeling

In addition to analyses undertaken to further explore effect of body weight on lecanemab exposure (AUC), a separate analysis was conducted to evaluate the potential clinical importance of exposure differences on efficacy and safety in subjects with low (51 kg, 5th percentile) and high (99 kg, 95th percentile) body weights.

The effect of body weight on efficacy as measured by reduction in brain amyloid load was evaluated by simulation analyses using PK/PD model for PET SUVr. The simulation results demonstrated comparable reduction in SUVr following a 720 mg SC weekly dose and 10 mg/kg biweekly IV dose for a typical 70 kg subject. Small differences in the reduction in PET SUVr for a subject with high (95th percentile) or low (5th percentile) body weight as demonstrated by simulation analysis are not considered to be clinically important. Thus, lecanemab exposure differences observed at the extremes of body weight are not expected to have a meaningful effect on lecanemab efficacy as defined by PET SUVr.

The effect of body weight on lecanemab safety defined as incidence of ARIA-E was also evaluated by simulation analysis based on PK/PD model.

Based on a PK/PD model for ARIA-E developed using data from the study of Example 4, lecanemab maximum serum concentration (Cmax) was a significant predictor of the risk of ARIA-E. Following single doses, subcutaneous administration of lecanemab resulted in approximately 4-fold lower Cmax compared to intravenous. Thus, the incidence of ARIA-E following SC administration is expected to be substantially lower compared to IV administration. This is confirmed by the model-based simulation analysis, wherein the incidence of ARIA-E in the first 6 months of treatment is predicted to be 2.1% (1.2%) for 720 mg weekly SC dose compared to 9% (3.7%) for 10 mg/kg biweekly IV dose for APOE4+ (APOE4−) subjects. ARIA-E incidence in subjects with high (95th percentile) or low (5th percentile) body weight was comparable to that in a subject with a reference 70 kg body weight as demonstrated by simulation analysis. The probability of experiencing ARIA-E following subcutaneous weekly administration is predicted to be lower than following intravenous biweekly and minimally affected by body weight.

In summary, exposure-response simulations using PET SUVr as a measure of efficacy and incidence of ARIA-E as a measure of safety demonstrated no clinically important effect of body weight, confirming that the proposed fixed subcutaneous dose can be administered to all subjects without regard for body weight.

### Amyloid PET Clearance

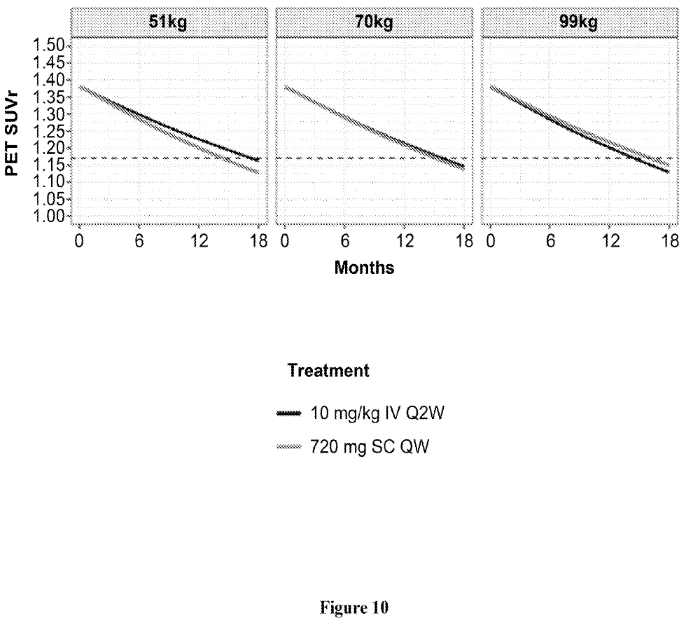

A dose of 10 mg/kg Q2W was compared to 720 mg QW SC for subjects with body weight of (a) 51 kg, (b) 70 kg, or (c) 99 kg. Amyloid PET clearance for IV and for SC were comparable and not affected by body weight following fixed SC dosing. See FIG. 10.

Small differences in the reduction in PET SUVr were observed between the three weight ranges (51 kg, 57-90 kg, and 99 kg) however they are not considered to be clinically important. That is, lecanemab exposure differences observed at the extremes of body weight are not expected to have a meaningful effect on lecanemab efficacy as defined by PET SUVr.

### Pharmacokinetics and Pharmacodynamics Modeling

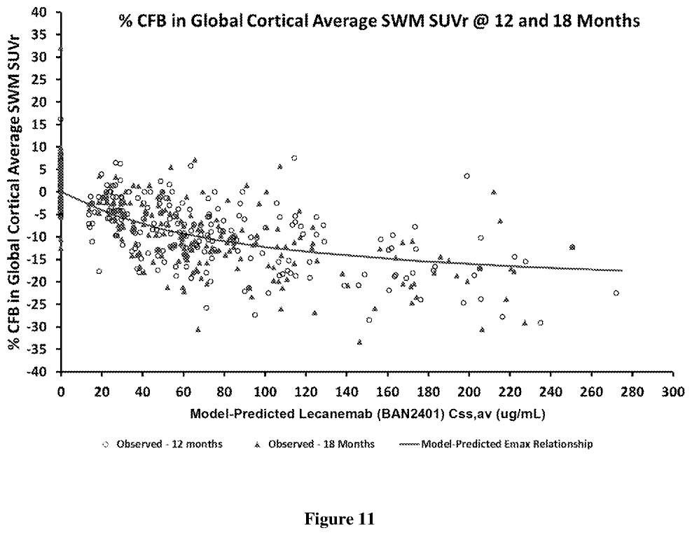

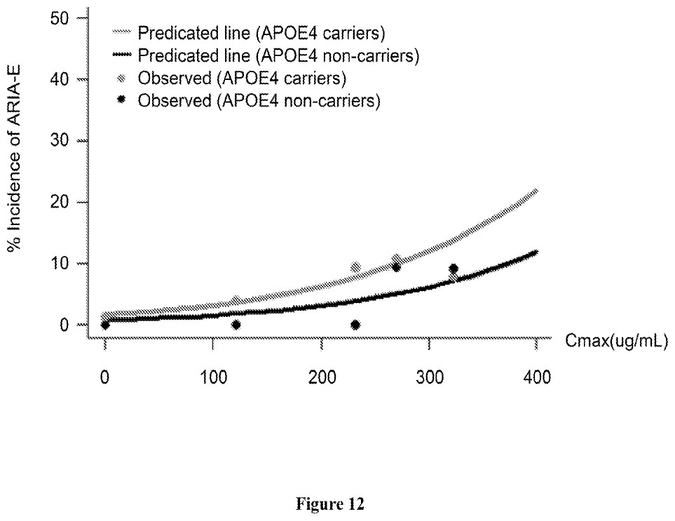

Based on the % change from baseline (CFB) in Global Cortical Average subcortical white matter (SWM) SUVr in subjects (data points at 12 months and 18 months), a predicted model establishes a correlation between PET SUVr and Cₐᵥₑ. A higher C_{ass, av} correlates to a greater amyloid reduction and clinical effect. See FIG. 11. The model also establishes a correlation between ARIA-E and Cmax. A lower Cmax correlates to a lower incidence of ARIA-E. See FIG. 12.

### Risk of ARIA-E

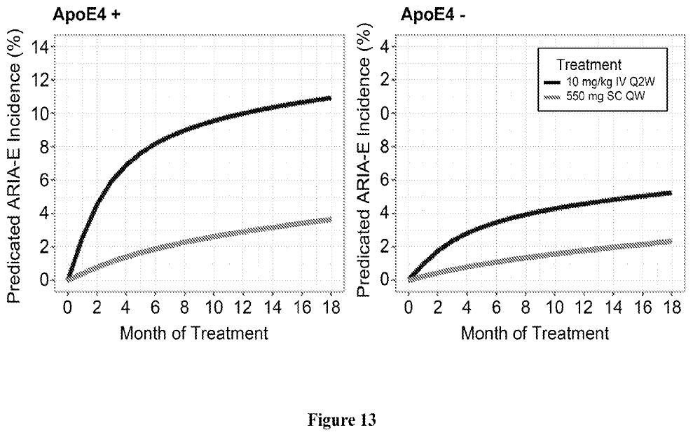

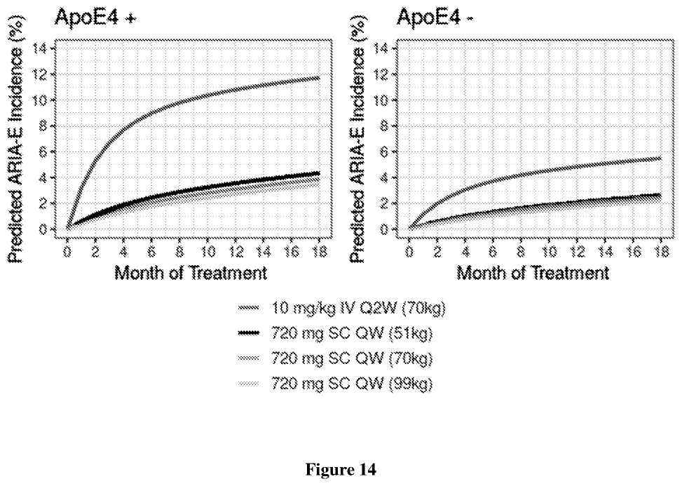

At steady-state, a model predicted Cmax following SC 550 mg QW and 720 mg QW is expected to be associated with a lower risk of ARIA-E compared to a 10 mg/kg IV treatment. A lower Cmax correlates to a lower incidence of ARIA-E. See FIG. 12. Predicted ARIA-E rates following modeled subcutaneous dosing (3.9%) are similar to lecanemab 5 mg/kg biweekly (3.6%) for ApoE4+ when administered intravenously. As shown below, ARIA-E incidence when the drug was administered subcutaneously was predicted to be lower than when the drug was administered intravenously. See Tables 15, 16, and 17 and FIGS. 13 and 14.

TABLE 15
Predicted ARIA-E incidence for SC (550 mg QW) and IV
treatments for ApoE4+ and ApoE4− subjects

| ARIA-E Incidence | ApoE+ | ApoE− |
|---|---|---|
| 10 mg/kg IV Q2W | 10.9 | 5.2 |
| 550 mg SC QW | 3.7 | 2.3 |

Tables 16 and 17. ARIA-E incidence for SC (720 mg QW) and IV (10 mg/kg
IV Q2W) treatments for ApoE4+ and ApoE4− subjects

| | ARIA-E Incidence (%) | | | | ARIA-E Incidence (%) | | |
|---|---|---|---|---|---|---|---|
| ApoE4+ | 6 mo | 12 mo | 18 mo | ApoE4− | 6 mo | 12 mo | 18 mo |
| 10 mg/kg IV Q2W | 9.0 | 10.8 | 11.7 | 10 mg/kg IV Q2W | 3.7 | 4.8 | 5.5 |
| 720 mg SC QW | 2.1 | 3.1 | 3.9 | 720 mg SC QW | 1.2 | 1.9 | 2.4 |

### Example 5: Second Subcutaneous Treatment Study Protocol

### Core Study

The “Core Study” is a multicenter, placebo-controlled, randomized, double-blind, open-label, parallel-group study that was conducted in subjects with Early AD (mild cognitive impairment [MCI] due to AD with intermediate likelihood/Prodromal AD or mild AD dementia) with confirmed amyloid pathology indicated by positive amyloid load. Amyloid pathology will be confirmed by amyloid PET assessment or CSF assessment of t tau/Aβ[1-42]. Approximately 1766 subjects will be randomized in the Core Study across 2 treatment groups (placebo and lecanemab IV 10 mg/kg, biweekly) according to a fixed 1:1 (placebo: lecanemab) schedule. Randomization across the 2 clinical subgroups (MCI due to AD/prodromal AD or mild AD dementia) will be reasonably balanced, such that not less than approximately 50% of total number of subjects will be in the MCI due to AD clinical subgroup. Subjects will be stratified according to clinical subgroup; presence or absence of ongoing approved AD treatment (e.g., acetylcholinesterase inhibitors [acetylcholinesterase inhibitors], memantine, or both); APOE4 status (i.e., APOE4 carriers or non-carriers); and geographical region.

Treatment in the Core Study will be for 18 months (a 1-month window and related scheduling changes will be applied if required for logistical purposes). This Core study for an individual subject is up to 24 months (up to 3 months for screening, 18 months of treatment, and a Follow-up Visit at 3 months post treatment).

### Test Drug:

For the intravenous infusions, lecanemab drug product will be supplied as a sterile aqueous solution comprising will be supplied as a sterile aqueous solution containing 100 mg/mL lecanemab, 50 mmol/L citrate, 350 mmol/L arginine, 0.05% polysorbate 80, pH 5.0, in glass vials containing 5 mL solution or supplied in a citrate-free formulation as a sterile aqueous solution containing 100 mg/mL lecanemab, 25 mmol/L histidine, 200 mmol/L arginine, 0.05% polysorbate 80, pH 5, in glass vials containing 5 mL solution. Lecanemab will be administered in normal saline as 60-minute intravenous infusions.

For the subcutaneous administration, lecanemab drug product was supplied in 2 mL vials containing 400 mg lecanemab, formulated at 200 mg/mL in 25 mmol/L histidine, 200 mmol/L arginine, 0.05% polysorbate 80, pH 5.0. Two vials will be provided for each weekly dose for a duration of at least 6 months. Each weekly dose of lecanemab 720 mg SC is composed of 2 consecutive injections of 360 mg (2×1.8 mL of 400 mg/2 mL SC formulation) each, which should be administered by a health care professional (HCP) into the abdomen, thigh, or upper arm, rotating within the assigned injection site in order to minimize pain, bruising or swelling. Lecanemab for subcutaneous administration should be drawn up into single use polypropylene syringes immediately before use and administered using a 25G subcutaneous needle over a period of approximately 15 seconds.

### Study Phases

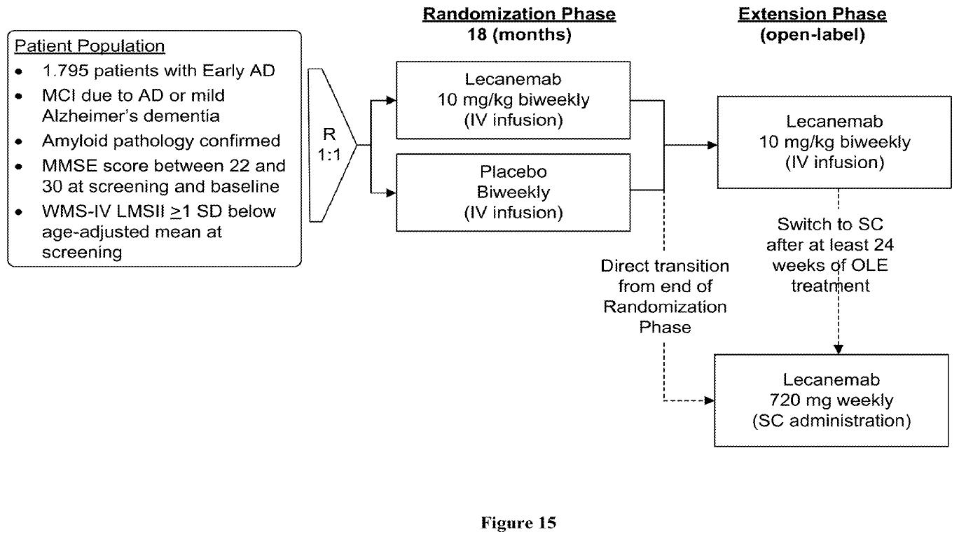

The study will consist of 3 phases: a Prerandomization Phase, a Randomization Phase, and an Extension Phase. The Randomization and Extension phases are shown in FIG. 15.

### Prerandomization Phase

The Prerandomization Phase may last up to 60 days, and will consist of a Screening Period and a Baseline Period.

### Randomization Phase

The Randomization Phase will consist of an 18-month Treatment Period and a 3 month Follow up Period (for those subjects who do not participate in the Extension Phase, discussed below). Subjects will be randomized at Visit 3 (Day 1) to receive either lecanemab (10 mg/kg, biweekly) or placebo (allocated 1:1; lecanemab:placebo) administered as a 60 minute intravenous infusion every 2 weeks.

### Extension Phase

An Extension Phase will be available for subjects who complete the full 18 months of placebo-controlled treatment in the Core Study and meet the inclusion/exclusion criteria of the Extension Phase. Subjects who participate in the Extension Phase will not complete the 3-month Follow-up Visit and will transition directly into the Extension Phase.

For subjects who participate in the Extension Phase, the Core Study period for an individual subject is approximately 20 months, which includes 2 months for screening and 18 months of treatment. Subjects who participate in the Extension Phase and discontinue treatment at any time will complete a 3-month Follow-up Visit. The Extension Phase will continue for up to 2 years, or until lecanemab becomes available, or until a positive risk-benefit assessment in this indication is not demonstrated, whichever comes first.

Subjects will receive open-label 10 mg/kg IV, biweekly treatment with lecanemab; or if participating in the optional subcutaneous (vial) substudy, weekly subcutaneous injections of 720 mg, administered as 2 consecutive injections of 360 mg (2×1.8 mL of 400 mg/2 mL SC formulation).

### Substudy in Extension Phase

A substudy in the Extension Phase will be conducted to explore subcutaneous administration of lecanemab and will evaluate the safety and tolerability, pharmacokinetics, immunogenicity, and effect on amyloid PET and on plasma biomarkers (such as or example p-tau181) of lecanemab, when administered subcutaneously in subjects previously treated only with placebo and in subjects previously treated with intravenous lecanemab.

The substudy is optional. Subjects who wish to continue on intravenous treatment during the Extension Phase may choose to do so.

Eligible for this substudy will be subjects who complete the Core Study, which can include subjects previously treated only with placebo before starting subcutaneous lecanemab in the Extension Phase and subjects previously treated with intravenous lecanemab. Subjects located in the US and Japan, who are eligible for entry to the Extension Phase, will also be eligible to participate in the optional subcutaneous (vial) substudy if it aligns with the recruitment window for this substudy. Subjects that have not yet started the Extension Phase can begin open-label treatment directly on the subcutaneous (vial) substudy upon completion of the Core Study and must agree to participate in or continue in the amyloid PET substudy. Subjects can also enter the subcutaneous (vial) substudy after 6 months of intravenous treatment in the Extension Phase.

Subjects participating in this substudy will be randomly assigned an injection site, which will be either the abdomen, the thigh, or the upper arm, with a fixed 1:1:1 schedule at each enrollment point (Visit 42 or Visit 56). Each consecutive injection should be rotated within the assigned injection site, using both sides of the body if needed.

Subjects in the subcutaneous (vial) substudy may revert to biweekly intravenous administration of lecanemab following approval by the Medical Monitor. In this case, the subject will remain on biweekly intravenous administration of lecanemab for the remainder of the study Extension Phase (lecanemab 10 mg/kg IV biweekly for up to 24 months [2 years] or until the drug is commercially available in the country where the subject resides, or the benefit to risk ratio from treatment with lecanemab is no longer considered favorable, whichever comes first).

Additionally, subjects who have taken part in the subcutaneous vial substudy will be provided the option to enroll in the subcutaneous AI (autoinjector) study after at least 6 months in the subcutaneous vial substudy. The subcutaneous AI substudy will look at subcutaneous administration using an AI device, which may be administered by a non-HCP (health care professional such as the subject, study partner, or a family member) at the investigator's discretion and only after the required training has been completed. The minimum period of initial AI training for non-HCP users will be 2 weeks and will take place across 2 consecutive study drug administration visits in clinic. If there is no suitable non-HCP to administer study drug using the AI device, study drug administration can be performed by a HCP. Subjects in the subcutaneous vial or AI substudies will have weekly study drug administration. For the subcutaneous vial substudy, vital signs, prior/concomitant medication assessment, and AE assessment must be performed every time study drug is administered. For the subcutaneous AI substudy, subjects are to come into the clinic at every visit at which clinical assessments are performed. At these visits injection technique will also be assessed. At AI dispensing visits, vital signs, prior/concomitant medication assessment, and AE assessment must also be undertaken.

The AI device is an automated, disposable 2.25 mL AI device consisting of a housing with a content viewing window, a spring activated mechanism and an integrated needle safety feature. The device contains a 2.25 mL prefilled plastic syringe with a tapered needle, rigid needle cover, and stopper, prefilled with 1.8 mL of 200 mg/mL lecanemab solution. The solution appears as a colorless to pale yellow liquid. The AI is ready to use and does not require any further assembly. The devices will be supplied in cartons, with each carton containing 2 devices.

Subjects participating in the subcutaneous AI substudy, subjects will receive 2 consecutive subcutaneous injections of a fixed dose (720 mg) of BAN2401 on a weekly basis, administered using an AI device. This will be dispensed in a pack of 2 AI devices. Since each AI device has a set amount of study drug 1.8 mL (360 mg BAN2401); therefore, both AI devices need to be administered for a full dose of study drug (720 mg). The AI device can be administered in the abdomen or thigh (for self-administration or if someone else is giving the injection) or the upper arm (if someone else is giving the injection; refer to AI instructions for use for full details).

### Follow-Up Visit

A Follow-up Visit will take place 3 months after the last dose of study drug.

Subjects may withdraw from the study or discontinue study drug for any reason during the Extension Phase. Subjects who withdraw from the study or discontinue study drug early must comply with the Early Termination Visit (within 7 days of the decision to discontinue from study drug) and the Follow-up Visit (3 months after the last dose of study drug) and may also have unscheduled visits for safety assessments when applicable. In the Extension Phase, subjects who discontinue study drug will not be required to return for each scheduled visit when clinical efficacy assessments are conducted. The study will end when the last visit assessment for the last subject of the Extension Phase has concluded.

### Pharmacokinetic Assessments

### Core Study and Extension Phase

Blood will be collected from subjects at Baseline (Tier 4) during the Prerandomization Phase before amyloid PET assessment, before the 1st dose of study drug at Visit 3, and at 6, 12, and 18 months of treatment to evaluate potential novel biomarkers of AD that may include amyloid isoforms, tau, and other protein biomarkers (e.g., NFL) for association with AD diagnosis and amyloid load. Similarly, biomarker discovery and validation may be performed along with samples from subjects with AD, to identify blood and genetic biomarkers which may be useful to predict subject PK and PD responses, treatment response, subject stratification or adverse effects related to lecanemab.

APOE4 genotyping will be conducted to allow stratification by APOE status (APOE4 carriers and non-carriers). APOE4 homozygous or heterozygous status will be used in the statistical analysis to determine the effects on treatment response and safety, including the development of Amyloid Related Imaging Abnormality (ARIA), which include vasogenic edema, microhemorrhages and superficial hemosiderosis. Remaining DNA from the APOE4 genotyping may be used to examine the role of DNA sequence variability in the absorption, distribution, metabolism, and elimination of lecanemab. Variations in lecanemab exposure or the occurrence of AEs observed in the study population may be evaluated by correlation of single nucleotide polymorphisms with PK, safety, or PD data.

Pharmacogenomic (PG) and biomarker samples obtained from participants of this study may be analyzed by global proteomic, metabolomic, or lipidomic and single or multiplex assays in an effort to identify predictive biomarkers for PK and PD. In addition, biomarkers identified in other lecanemab or AD clinical studies may also be assessed in samples collected from subjects enrolled in this study. [00341] vMRI imaging will be used to evaluate the effects of lecanemab on rates of atrophy in the EAD population to provide evidence for disease modification. All subjects will undergo a vMRI imaging sequence immediately following all safety MRI assessments. vMRI sequences also will be analyzed at the Screening Visit and at Visits 16, 29, and 42 (6, 12, and 18 months of treatment) during the Core Study. vMRI sequence collections will occur at all safety MRI assessments during the Extension Phase. Total hippocampal, whole brain, and ventricular volumes will be assessed.

CSF concentrations of AD-related biomarkers (including but not limited to Aβ[1-42], Aβ[1-40], neurogranin, NFL, t tau and p tau) will be measured in consenting subjects at Baseline and at 12 and 18 months of treatment.

In the Core Study, blood samples will be collected from all subjects for determination of serum lecanemab levels at approximately 12-week intervals. Subjects who withdraw from the study or discontinue study drug early will have blood samples collected at the Early Termination Visit (within 7 days of the decision to discontinue from study drug) and the Follow-up Visit (3 months after the last dose of study drug).

In the Extension Phase, blood samples will be collected at Week 9 Visit 42, 47, Visit 50, and every 3 months thereafter during the 1st year of the Extension Phase, and every 6 months thereafter during the 2nd year of the Extension Phase, at the Early Termination Visit when applicable, and at the Follow-up Visit that takes place 3-months after the last dose of study drug.

A population PK approach will be used to characterize the PK of lecanemab. The effect of covariates (e.g., including but not limited to, demographics, concomitant medications, ADA development, and study drug formulation) on lecanemab PK will be evaluated. The PK model will be parameterized for clearance (CL) and volumes of distribution. Derived exposure parameters such as AUC and average concentration (Cav) will be calculated from the model using the individual posterior estimate of CL and dosing history.

### Subcutaneous Substudy

Subjects participating in the optional subcutaneous substudy will require additional blood samples to be taken for serum PK.

### Safety Assessments

### Core Study and Extension Phase

During the Extension Phase, safety assessments will continue to be monitored. AEs, including SAEs and study-specific AEs, will be identified, assessed, and collected. Vital signs will be assessed when study drug is administered both predose and after infusion. Hematology, blood chemistry, and urine laboratory test values will be monitored every 6 months.

All subjects will be assessed using clinical laboratory tests, safety MRIs, vMRIs, amyloid PET assessments, tau PET assessments, and CSF sampling. All subjects will follow the same safety MRI schedule as in the Core Study for the first 6 months of treatment in the Extension Phase for amyloid-related imaging abnormality edema/effusion (ARIA E) monitoring (at 9 weeks, 13 weeks, and 6 months after the start of the Extension). Safety MRIs will be conducted every 6 months thereafter until the end of the Extension Phase. Volumetric MRI assessments will be collected following all safety MRI assessments and will be analyzed at 24, 30, 36, and 42 months in the Extension Phase.

Clinical assessments will be administered every 6 months in the morning (whenever possible) in the following order: MMSE, CDR-SB, and ADAS-cog14. All clinical assessments (MMSE, CDR-SB, and ADAS-cog14) must be completed on the same day. All clinical assessments must be completed in the morning whenever possible, or consistently at approximately the same time of day during the study. EQ-5D-5L, QOL-AD, ADCS MCI ADL, and Zarit Burden Interview will be completed following the completion of the ADAS-cog 14.

Blood for serum PK will be collected at Visit 42, Visit 47, Visit 50, Week 9 and every 3 months thereafter during the 1st year of the Extension Phase, and every 6 months thereafter during the 2nd year of the Extension Phase, at the Early Termination Visit when applicable, and at the Follow up Visit that takes place 3 months after the last dose of study drug.

Amyloid PET will be collected for those who consent to the longitudinal PET substudy in the Core Study at 30 and 42 months in the Extension Phase, while CSF will be collected for those who consent to the longitudinal CSF substudy in the Core Study at 30 and 42 months in the Extension Phase. Tau PET will be collected for those who consent to the longitudinal tau PET substudy in the Core Study at 30 and 42 months in the Extension Phase.

### Pharmacodynamic, Pharmacogenomic, and Other Biomarker Assessments

Blood samples for genotyping of APOE4 will be obtained from subjects at (Screening). A blood sample will also be taken during Prerandomization for additional AD diagnostics.

### Other Assessments

Subjects who consented to the amyloid PET, tau PET, and/or CSF substudies in the Core Study may continue these substudy evaluations. Amyloid PET will be collected for those who consent to the longitudinal amyloid PET substudy in the Core Study at 30 and 42 months in the Extension Phase, while CSF will be collected for those who consent to the longitudinal CSF substudy in the Core Study at 30 and 42 months in the Extension Phase. Tau PET will be collected for those who consent to the longitudinal tau PET substudy in the Core Study at 30 and 42 months in the Extension Phase. (revised per Amendment 08)

All subjects who choose to participate in the subcutaneous (vial) substudy and enter this substudy at the start of the Extension Phase (Week 1[Visit 42]) must have an amyloid PET scan in the 4 weeks before initiation of subcutaneous administration as the Baseline subcutaneous (vial) substudy amyloid PET scan; these subjects do not need to have participated in the Core Study amyloid PET substudy. Subjects entering the subcutaneous (vial) substudy after 6 months of intravenous treatment in the Extension Phase are not required to take part in the amyloid PET substudy but those who are participating in the amyloid PET substudy may continue in the amyloid PET substudy per the regular schedule of assessments.

### Subcutaneous (Vial) Substudy Endpoints

### Primary Endpoints:

Incidence of AEs and changes in vital signs, ECGs, laboratory safety tests, suicidality assessments, ADAs, and MRI safety parameters when lecanemab is administered subcutaneously by an HCP
Population PK parameters of lecanemab in serum, including but not limited, to AUC, Cav.

### Secondary Endpoints:

Incidence and timing of ADA onset, ADA titer, and other characteristics related to subject ADA status over the subcutaneous treatment period, and incidence and timing of neutralizing ADA (NAb) onset, NAb titer, and other characteristics related to subject NAb status over the subcutaneous treatment period
Change from substudy baseline in brain amyloid levels over the subcutaneous treatment period in subjects previously treated only with placebo before starting subcutaneous lecanemab in the OLE and in subjects previously treated with intravenous lecanemab
Proportion of subjects who convert from amyloid PET positive to amyloid PET negative by visual read, SUVR, and Centiloid scales over the subcutaneous treatment period in subjects previously treated only with placebo before starting subcutaneous lecanemab in the OLE and in subjects previously treated with intravenous lecanemab
Change from substudy baseline in plasma biomarkers (such as, for example, p-tau181) over the subcutaneous treatment period in subjects previously treated only with placebo before starting subcutaneous lecanemab in the OLE and in subjects previously treated with intravenous lecanemab

### Substudy Analysis Sets

The Extension Safety Analysis for the subcutaneous (vial) substudy Set (Extension-SC-SAS) is the group of subjects who received at least 1 dose of subcutaneously administered study drug (vial and syringe) over the subcutaneous treatment period.

The Extension PK Analysis Set for the subcutaneous (vial) substudy is the group of subjects who received at least 1 dose of study drug during the Core Study with at least 1 quantifiable lecanemab serum (analysis set for serum) or CSF (analysis set for CSF) concentration with a documented subcutaneous (vial and syringe) dosing history during the Extension Phase.

The Extension PD Analysis Set for the subcutaneous (vial) substudy is the group of subjects who received at least 1 dose of subcutaneously administered study drug (vial and syringe) over the subcutaneous treatment period and had sufficient PD data to derive at least 1 PD parameter (had baseline and at least 1 postdose assessment) during that period.

### Example 6: Autopsy Findings in Subjects with Alzheimer's Disease Who Received Long-Term Treatment with Lecanemab (BAN2401)

A patient (˜85-year old) was enrolled in the Core study described above. The patient, previously diagnosed with mild cognitive impairment after 3 years of having mild memory problems, was on active treatment at 10 mg/kg q 4 weeks (every 4 weeks) for 79 weeks, then had 98 weeks without treatment, followed by extension phase with 10 mg/kg every 2 weeks for 94 weeks. The patient developed behavioral symptoms, stopped treatment, and died 12 weeks later, 9 years after first symptoms developed.

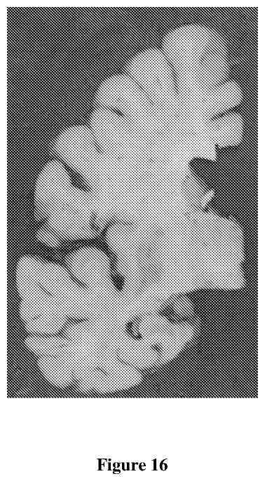

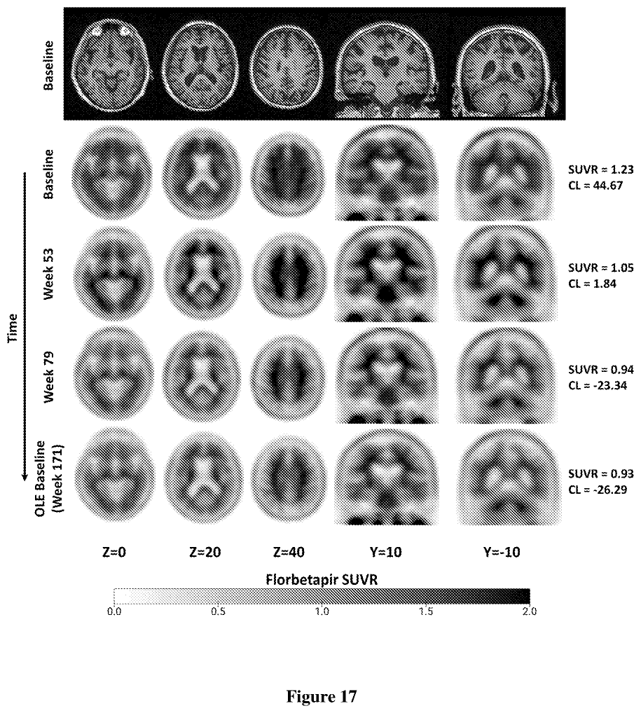

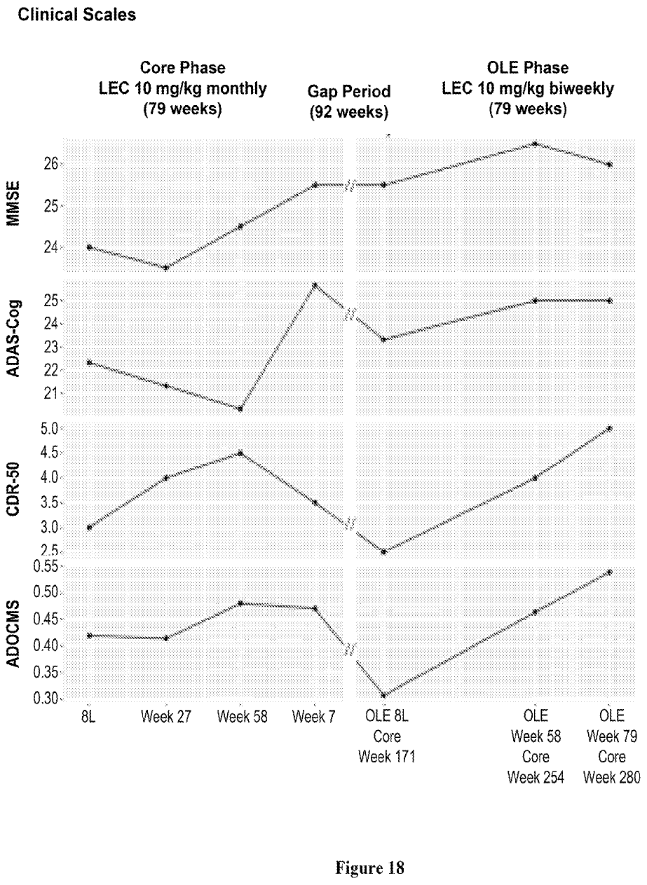

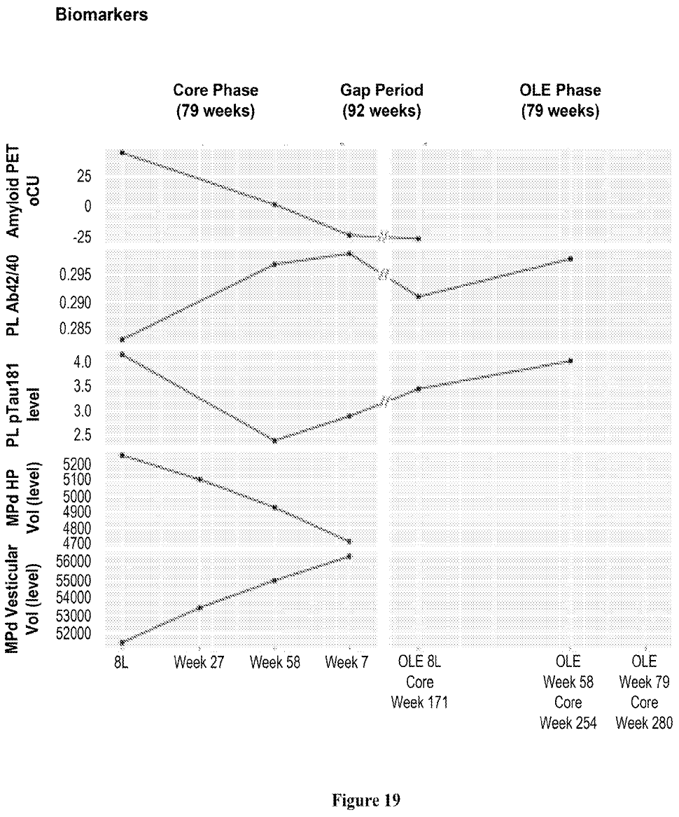

An autopsy was performed. Brain showed moderate atrophy (brain weight 1052 gm). See FIG. 16. No infarcts or bleeds were present. The brain tissue was sampled from multiple regions (frontal, parietal, occipital, hippocampus, brainstem), and full neuropathological evaluation was performed with histological (LH&E, Bielschowsky, thioflavine) and immunohistochemical stains for pathological proteins (tau [AT8], beta-amyloid [6E10], a-synuclein, TDP43) and astroglial and microglial responses (GFAP, CD68). See Table 18, and FIGS. 17, 18 and 19.

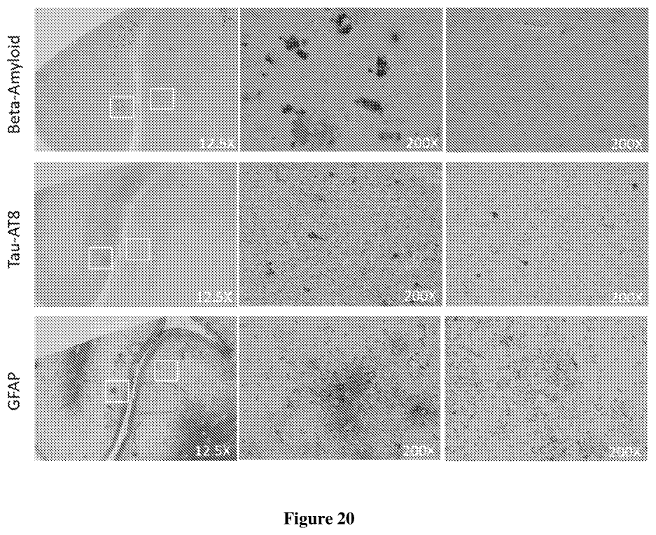

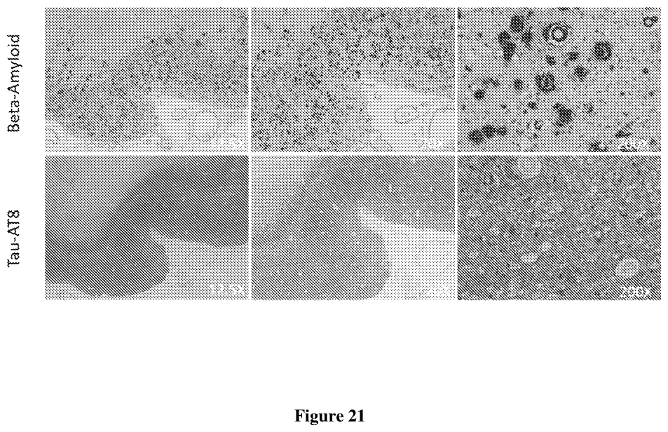

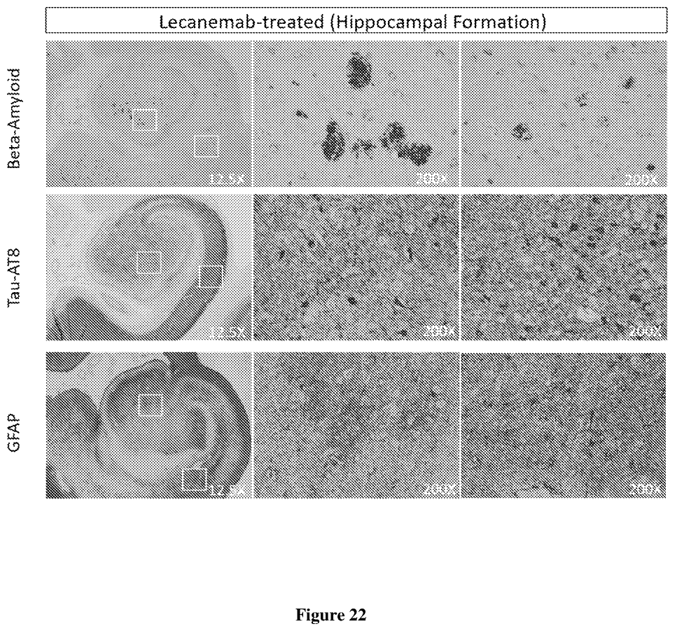

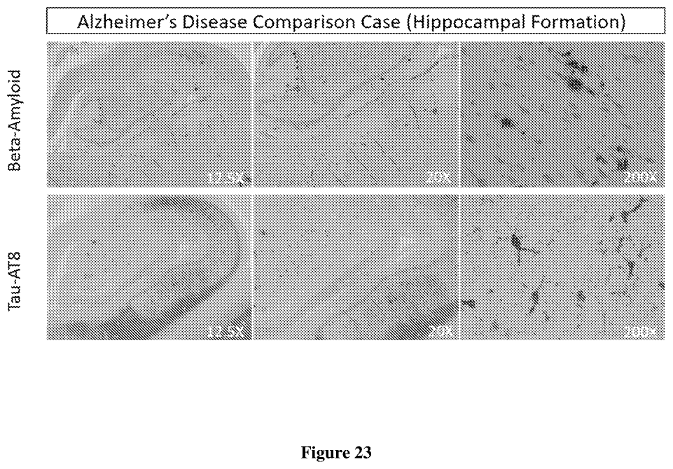

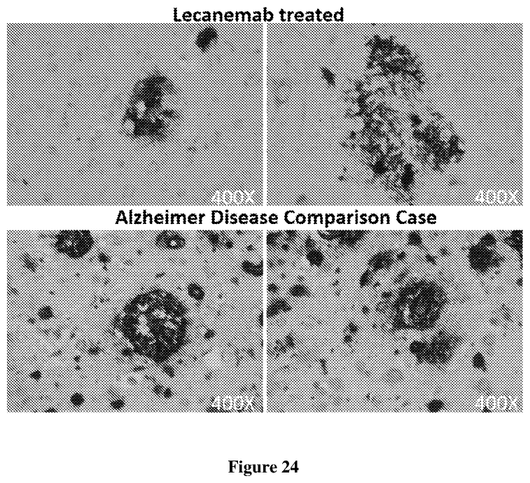

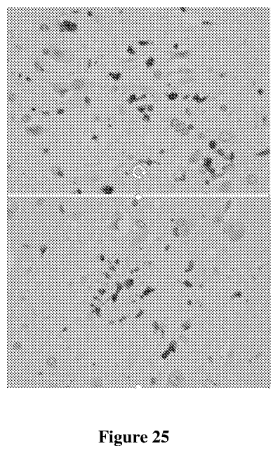

Principal findings were very sparse amyloid deposits—very little diffuse amyloid, and only sparse and patchy plaques. See FIGS. 20-23. Amyloid plaques in lecanemab treated are less homogeneous and less dense. See FIG. 24. Amyloid patches mostly confined to CA4 region of the hippocampus. See FIG. 22. Tau staining is present—but CA4 tangles are sparse. See FIGS. 20 and 22. Neuritic plaques were present in neocortex and allocortex, but overall, relatively sparse, despite a few areas of more clustered plaques. Neurofibrillary threads were present throughout all cortical regions. Neurofibrillary tangles were widely present, but were not in high densities. Modest focal amyloid angiopathy was present. Mild granulovacuolar degeneration was present. CD68 staining for microglia was present around amyloid material. See FIG. 25. Minor TDP43 cytoplasmic staining was present only in amygdala and entorhinal cortex. Lewy Bodies were present only in the amygdala.

TABLE 18
Topographic distribution of Alzheimer's findings

| | Beta-Amyloid | | |
|---|---|---|---|
| Brain Regions | Plaques*** | Phospho-Tau** | Bielschowsky |
| Superior Frontal Cortex (BA8, BA9) | + | ++(NT, T), +(NP) | + |
| Posterior Frontal Cortex (BA4) | +/− | ++(NT, T) | +/− |
| Parietal Cortex (BA1, BA3, BA5, BA40) | − | ++(NT, T) | − |
| Calcarine Cortex (BA17, BA18 & BA31) | ++ | ++(NT, T, NP) | ++ |
| Hippocampal Formation, LGB, CN tail | +++(CA4, CA1) +/−(EG) | +++(NT, T, NP) | +++(CA4, CA1) +/−(EC) |
| CAP (Caudate, Putamen, Accumbens) | − | ++(NT) | − |
| GP and Putamen with Claustrum | − | n/a | n/a |
| Amygdala Proper | (−) proper | +++(NT, T) | − |
| Amygdala | +++ | +++(NT < T, NP) | +++ |
| Thalamus (level of anterior nucleus) | − | n/a | n/a |
| Midbrain | − | ++(NT), +(T) | n/a |
| Upper Pons (level of locus coeruleus) | n/a | +(NT < T) | n/a |
| Cerebellum (with dentate nucleus) | (−) | n/a | n/a |
| Subthalamic Nucleus with Anterior Thalamus | (−) | n/a | n/a |

* +++, diffuse;
++, moderate;
+, sparse/local;
−/−rare;
**NT, neurofibrillary threads;
T, neurofibrillary tangles;
NP, neuritic plaques;
+++ most dense core plaques.

The neuropathological findings in this case with a 9-year history of Alzheimer's symptoms are most notable for a marked paucity of diffuse plaques, and a variable but overall, very low burden of neuritic plaques. Those plaques that were present had a “moth-eaten” appearance. There was also a lack of marked amyloid angiopathy. Neurofibrillary pathology is more extensive and marked in threads than tangles. Tau PET in phase 3 CLARITY AD study will assess whether amyloid clearance slows tau pathology. The presence of topographically extensive neurofibrillary pathology in the setting of near absence of diffuse amyloid and only scattered neuritic amyloid is very uncommon in typical AD: in the NACC neuropathology dataset, only 2% of brains with Braak B2 or B3 show Thal stage A0 or A1. The neuropathological findings are consistent with the florbetapir PET scans which show marked reduction of tracer uptake with lecanemab treatment. The results of the neuropathological examination thus support lecanemab-induced removal of fibrillar amyloid—both diffuse and neuritic.

### SEQUENCE LISTING

TABLE 19
Amino acid sequences of mAb variable regions

| | IgG | SEQ ID | |
|---|---|---|---|
| mAb | chain | NO | Amino acid sequence |
| Lecanemab | Heavy | 1 | EVQLVESGGGLVQPGGSLRLSCSA |
| | chain | | SGFTFSSFGMHWVRQAPGKGLEWV |
| | | | AYISSGSSTIYYGDTVKGRFTISR |
| | | | DNAKNSLFLQMSSLRAEDTAVYYC |
| | | | AREGGYYYGRSYYTMDYWGQGTTV |
| | | | TVSS |
| Lecanemab | Light | 2 | DVVMTQSPLSLPVTPGAPASISCR |
| | chain | | SSQSIVHSNGNTYLEWYLQKPGQS |
| | | | PKLLIYKVSNRFSGVPDRFSGSGS |
| | | | GTDFTLRISRVEAEDVGIYYCFQG |
| | | | SHVPPTFGPGTKLEIK |

TABLE 20
Amino acid sequences of mAb constant regions

| | IgG | | SEQ ID | |
|---|---|---|---|---|
| mAb | chain | Class | NO | Amino acid sequence |
| Lecanemab | Heavy | IgG1 | 3 | ASTKGPSVFPLAPSSKSTSGGTA |
| | chain | | | ALGCLVKDYFPEPVTVSWNSGAL |
| | | | | TSGVHTFPAVLQSSGLYSLSSVV |
| | | | | TVPSSSLGTQTYICNVNHKPSNT |
| | | | | KVDKRVEPKSCDKTHTCPPCPAP |
| | | | | ELLGGPSVFLFPPKPKDTLMISR |
| | | | | TPEVTCVVVDVSHEDPEVKFNWY |
| | | | | VDGVEVHNAKTKPREEQYNSTYR |
| | | | | VVSVLTVLHQDWLNGKEYKCKVS |
| | | | | NKALPAPIEKTISKAKGQPREPQ |
| | | | | VYTLPPSREEMTKNQVSLTCLVK |
| | | | | GFYPSDIAVEWESNGQPENNYKT |
| | | | | TPPVLDSDGSFFLYSKLTVDKSR |
| | | | | WQQGNVFSCSVMHEALHNHYTQK |
| | | | | SLSLSPGK |
| Lecanemab | Light | kappa | 4 | RTVAAPSVFIFPPSDEQLKSGTA |
| | chain | | | SVVCLLNNFYPREAKVQWKVDNA |
| | | | | LQSGNSQESVTEQDSKDSTYSLS |
| | | | | STLTLSKADYEKHKVYACEVTHQ |
| | | | | GLSSPVTKSFNRGEC |

TABLE 21
Amino acid sequences of mAb
complementarity determining regions

| | IgG | SEQ ID | Amino acid |
|---|---|---|---|
| mAb | chain | NO | sequence |
| Lecanemab | HCDR1 | 5 | SFGMH |
| | HCDR2 | 6 | YISSGSSTIYYGDTVKG |
| | HCDR3 | 7 | EGGYYYGRSYYTMDY |
| Lecanemab | LCDR1 | 8 | RSSQSIVHSNGNTYLE |
| | LCDR2 | 9 | KVSNRFS |
| | LCDR3 | 10 | FQGSHVPPT |

TABLE 22
Amino acid sequences of Amyloid β

| | SEQ ID | |
|---|---|---|
| Amyloid β | NO | Amino acid sequence |
| Amyloid β | 11 | DAEFRHDSGYEVHHQKLVFFA |
| 1-42 | | EDVGSNKGAIIGLMVGGVVIA |
| Amyloid β | 12 | DAEFRHDSGYEVHHQKLVFFA |
| 1-40 | | EDVGSNKGAIIGLMVGGVV |

SEQ ID NO: 13
heavy chain:
evqlvesggglvqpggslrlscsasgftfssfgmhwvrqapgkglewvay
issgsstiyygdtvkgrftisrdnaknslflqmsslraedtavyycareg
gyyygrsyytmdywgqgttvtvssastkgpsvfplapsskstsggtaalg
clvkdyfpepvtvswnsgaltsgvhtfpavlqssglyslssvvtvpsssl
gtqtyicnvnhkpsntkvdkrvepkscdkthtcppcpapellggpsvflf
ppkpkdtlmisrtpevtcvvvdvshedpevkfnwyvdgvevhnaktkpre
eqynstyrvvsvltvlhqdwlngkeykckvsnkalpapiektiskakgqp
repqvytlppsreemtknqvsltclvkgfypsdiavewesngqpennykt
tppvldsdgsfflyskltvdksrwqqgnvfscsvmhealhnhytqkslsl
spgk
light chain:
dvvmtqsplslpvtpgapasiscrssqsivhsngntylewylqkpgqspk
lliykvsnrfsgvpdrfsgsgsgtdftlrisrveaedvgiyycfqgshvp
ptfgpgtkleikrtvaapsvfifppsdeqlksgtasvvcllnnfypreak
vqwkvdnalqsgnsqesvteqdskdstyslsstltlskadyekhkvyace
vthqglsspvtksfnrgec

1. A method of treating Alzheimer's disease comprising subcutaneously administering to a subject in need thereof 400 mg to 1500 mg, such as 400 mg to 800 mg, of an anti-Aβ protofibril antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

2. A method of delaying clinical decline comprising subcutaneously administering to in a subject in need thereof 400 mg to 1500 mg, such as 400 mg to 800 mg of an anti-Aβ protofibril antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

3. A method of reducing brain amyloid level comprising subcutaneously administering to a subject in need thereof 400 mg to 1500 mg, such as 400 mg to 800 mg of an antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

4. A method of converting an amyloid positive subject to amyloid negative comprising subcutaneously administering to the subject 400 mg to 1500 mg, such as 400 mg to 800 mg of an antibody comprising three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

5. The method according to any one of claims 1 to 4, wherein the subject has been diagnosed as having early Alzheimer's disease.

6. The method according to any one of claims 1 to 4, wherein the subject has been diagnosed as having Alzheimer's disease.

7. The method according to any one of claims 1 to 4, wherein the subject is at risk of developing Alzheimer's disease.

8. The method according to any one of claims 1 to 7, wherein the anti-Aβ protofibril antibody is administered once weekly.

9. The method according to any one of claims 1 to 8, wherein the anti-Aβ protofibril antibody is administered at a dose of 400 mg to 500 mg, 500 mg to 600 mg, 600 mg to 700 mg, or 700 mg to 800 mg.

10. The method according to any one of claims 1 to 9, wherein the anti-Aβ protofibril antibody is administered at a dose of 440 mg, 580 mg, or 720 mg.

11. The method according to any one of claims 1 to 10, wherein the anti-Aβ protofibril antibody comprising a heavy chain complementarity variable region comprising an amino acid sequence of SEQ ID NO: 1, and a light chain variable region comprising an amino acid sequence of SEQ ID NO: 2.

12. The method according to any one of claims 1 to 11, wherein the subject is ApoE4-positive.

13. The method according to any one of claims 1 to 12, wherein the anti-Aβ protofibril antibody is comprised in a pharmaceutical composition in the form of pre-filled syringe or an autoinjector.

14. A method of treating Alzheimer's Disease comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

15. A method of treating preclinical Alzheimer's Disease comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

16. A method of delaying clinical decline in a subject having Alzheimer's disease comprising subcutaneously administering to the subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

17. A method of reducing brain amyloid level in a subject comprising subcutaneously administering to the subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

18. A method of converting a subject from amyloid positive to negative comprising subcutaneously administering to a subject in need thereof an aqueous pharmaceutical composition comprising:
(a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
(b) 100 mM to 400 mM arginine and/or arginine hydrochloride;
(c) 0.01% w/v to 0.1% w/v polysorbate 80; and
(d) a pharmaceutically acceptable buffer;
wherein the pharmaceutical composition has a pH ranging from 4.5 to 5.5.

19. A method of delaying the pathophysiological and clinical progression of Alzheimer's Disease comprising subcutaneously administering to a subject in need thereof an aqueous pharmaceutical composition comprising:
a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
b) 100 mM to 400 mM arginine and/or arginine hydrochloride;
c) 0.01% w/v to 0.1% w/v polysorbate 80; and
d) a pharmaceutically acceptable buffer;
wherein the pharmaceutical composition has a pH ranging from 4.5 to 5.5.

20. A method of preventing Alzheimer's Disease comprising subcutaneously administering to a subject in need thereof an aqueous pharmaceutical composition comprising:
a) 200 mg/mL of an anti-Aβ protofibril antibody or a fragment thereof comprising a heavy chain variable region comprising the amino acid sequence of SEQ ID NO: 1 and a light chain variable region comprising the amino acid sequence of SEQ ID NO: 2;
b) 100 mM to 400 mM arginine and/or arginine hydrochloride;
c) 0.01% w/v to 0.1% w/v polysorbate 80; and
d) a pharmaceutically acceptable buffer;
wherein the pharmaceutical composition has a pH ranging from 4.5 to 5.5.

21. The method of any one of claims 15 to 20, wherein the subject has intact cognition.

22. The method of any one of claims 15 to 21, wherein the subject has elevated amyloid.

23. The method of any one of claims 15 to 21, wherein the subject has intermediate amyloid.

24. The method of any one of claims 15 to 23, wherein the subject is subcutaneously administered one injection of the pharmaceutical composition weekly from week 0 though week 8, followed by two injections of the pharmaceutical composition weekly from week 10 through week 96, followed by two injections of the pharmaceutical composition.

25. The method of any one of claims 15 to 23, wherein the subject is subcutaneously administered the pharmaceutical composition comprising 440 mg, 580 mg, or 720 mg of the anti-Aβ protofibril antibody weekly from week 0 through week 216.

26. The method of any one of claims 15 to 23, wherein the subject is subcutaneously administered one injection of the pharmaceutical composition every two weeks from week 0 through week 4, followed by two injections of the pharmaceutical composition every two weeks from week 6 through week 212.

27. The method of any one of claims 15 to 23, wherein the subject is administered the pharmaceutical composition weekly for at least two years after administration of the first dose of the pharmaceutical composition to the subject.

28. The method of any one of claims 15 to 27, wherein the subject is administered the pharmaceutical composition for at least 4 years.

29. The method of any one of claims 15 to 29, wherein the subject is administered a maintenance dose of the pharmaceutical composition.

30. The method of any one of claims 15 to 30, wherein the subject is monitored for amyloid accumulation and development of neurofibrillary tangles based on a PET scan for tau, plasma and/or CSF biomarkers.

31. The method of any one of claims 15 to 23, wherein the subject is subcutaneously administered one injection of the pharmaceutical composition weekly from week 0 through week 8, followed by two injections of the pharmaceutical composition weekly from week 10 through week 96 weeks, followed by two injections of the pharmaceutical formulation every two weeks from week 98 through week 216.

32. The method of any one of claims 15 to 23, wherein the subject is subcutaneously administered two injections of the pharmaceutical composition from week 8 through week 94 and/or from week 98 through week 216.

33. The method of any one of claims 15 to 23, wherein the subject is subcutaneously administered the pharmaceutical composition comprising 440 mg, 580 mg, or 720 mg of the anti-Aβ protofibril antibody weekly from week 0 through week 96, followed by administration of said pharmaceutical composition every two weeks from week 98 through week 216.

34. The method of any one of claims 15 to 23, wherein the subject is subcutaneously administered one injection of the pharmaceutical composition every two weeks from week 0 through to week 8, followed by two injections of the pharmaceutical composition every two weeks from week 10 through to week 216.

35. The method of any one of claims 15 to 23, wherein the subject is subcutaneously administered the pharmaceutical composition comprising 440 mg, 580 mg, or 720 mg of the anti-Aβ protofibril antibody every two weeks from week 10 to through week 216.

36. The method of claim 35, wherein the subject is subcutaneously administered the pharmaceutical composition comprising 440 mg, 580 mg, or 720 mg of the anti-Aβ protofibril antibody every two weeks from week 10 to through week 212.

37. The method any one of claims 1 to 36, wherein the subject is 65 to 80 years old.

38. The method any one of claims 1 to 37, wherein the subject is 55 to 64 years old and has at least one risk factor chosen from:
(i) a first degree relative diagnosed with dementia onset before age 75;
(ii) at least one apolipoprotein E4 variant (APOE4) allele; and
(iii) elevated brain amyloid according to PET or cerebrospinal fluid (CSF) testing prior to said administration.

39. The method of any one of claims 1 to 38, wherein the subject has a Global Clinical Dementia Rating (CDR) score of 0 at prior to said administration.

40. The method of any one of claims 1 to 39, wherein the subject has a Mini-Mental State Examination (MMSE) score greater than or equal to 27, with educational adjustments, prior to said administration.

41. The method of any one of claims 1 to 40, wherein the subject has a Wechsler Memory Scale-Revised Logical Memory subscale II (WMS-R LM II) score prior to said administration of at least one standard deviation below age-adjusted mean in the WMS-IV LMII of less than or equal to 15 for a subject of age ranging from 50 to 64 years, of less than or equal to 12 for a subject of age ranging from 65 to 69 years, of less than or equal to 11 for a subject of age ranging from 70 to 74 years, of less than or equal to 9 for a subject of age ranging from 75 to 79 years, and of less than or equal to 7 for a subject of age ranging from 80 to 90 years.

42. The method any one of claims 24, 26, 31, 32, or 34, wherein the volume of the injection is 1.1 mL, 1.4 mL, or 1.8 mL.
